(19) United States
(12) Patent Application Publication
(10) Pub. No.: US 2016/0175085 A1
(43) Pub. Date: Jun. 23, 2016

(54) ENHANCED FLUOROGENIC ENDOLUMINAL FILTER STRUCTURE

(71) Applicant: VOLCANO CORPORATION, San Diego, CA (US)

(72) Inventors: Eric Johnson, Woodside, CA (US); Jeremy Stigall, Carlsbad, CA (US); Christopher Leblanc, Carlsbad, CA (US)

(21) Appl. No.: 14/958,476

(22) Filed: Dec. 3, 2015

Related U.S. Application Data
(60) Provisional application No. 62/095,473, filed on Dec. 22, 2014.

(51) Int. Cl. A61F 2/01

(52) U.S. Cl. CPC A61F 2/013; A61F 2002/016; A61F 2002/011

(57) ABSTRACT

An endoluminal filter including a first support member having a first end and a second end and a second support member attached to the first end of the first support member or the second end of the first support member and forming a crossover with the first support member. The endoluminal filter also includes a material capture structure extending between the first and second support members, the crossover and the first end or the second end of the first support member and at least one tissue anchor on the first support member or the second support member. The material capture structure can be formed by, for example, joining intersecting filaments, laser cutting a sheet of material, or additive manufacturing. A perimeter of the material capture structure can include attachments to join the material capture structure to the first or second support member.

### CROSS REFERENCE TO RELATED APPLICATIONS

This application claim priority to and the benefit of U.S. Provisional Application No. 62/095,473 filed Dec. 22, 2014, the content of which is incorporated herein in its entirety.

This application is also related to the following applications: U.S. Publication No. 2013/0012981, entitled “ENDOLUMINAL FILTER WITH FIXATION”, filed Jul. 19, 2012; International Publication No. WO 2013/106746, entitled “ENDOLUMINAL FILTER WITH FIXATION”, filed Jan. 11, 2013; U.S. Provisional Application No. 61/785,204, filed Mar. 14, 2013; and U.S. Provisional Application No. 61/785,955, filed Mar. 14, 2013.

### INCORPORATION BY REFERENCE

All publications and patent applications mentioned in this specification are herein incorporated by reference to the same extent as if each individual publication or patent application was specifically and individually indicated to be incorporated by reference.

### FIELD

This invention relates generally to devices and methods for providing filtration of debris within a body lumen. More particularly, the invention provides a retrievable filter placed percutaneously in the vasculature of a patient to prevent passage of emboli. Additionally, embodiments of the invention provide a filter that can be atraumatically positioned and subsequently removed percutaneously from a blood vessel using either end of the filter.

### BACKGROUND

Embolic protection is utilized throughout the vasculature to prevent the potentially fatal passage of embolic material in the bloodstream to smaller vessels where it can obstruct blood flow. The dislodgement of embolic material is often associated with procedures which open blood vessels to restore natural blood flow such as stenting, angioplasty, arthrectomy, endarterectomy or thrombectomy. Used as an adjunct to these procedures, embolic protection devices trap debris and provide a means for removal for the body.

One widely used embolic protection application is the placement of filtration means in the vena cava. Vena cava filters (VCF) prevent the passage of thrombus from the deep veins of the legs into the blood stream and ultimately to the lungs. This condition is known as deep vein thrombosis (DVT), which can cause a potentially fatal condition known as pulmonary embolism (PE).

The first surgical treatment for PE, performed by John Hunter in 1874, was femoral vein ligation. The next major advancement, introduced in the 1950's, was the practice of compartmentalizing of the vena cava using clips, suture or staples. While effective at preventing PE, these methods were associated with significant mortality and morbidity (see, e.g., Kinney TB, Update on inferior vena cava filters, JVIR 2003; 14:425-440, incorporated herein by reference).

A major improvement in PE treatment, in which venous blood flow was maintained, was presented by DeWesse in 1955. This method was called the “harp-string” filter, as represented in FIG. 1A and FIG. 1B, in which strands of silk suture 12 were sewn across the vena cava 11 in a tangential plane below the renal veins 13 to trap thrombus. Reported clinical results demonstrated the effectiveness of this method in preventing PE and maintaining caval patency. (see, e.g., DeWeese M S, A vena cava filter for the prevention of pulmonary embolism, Arch of Surg 1963; 86:852-868, incorporated herein by reference). Operative mortality associated with all of these surgical treatments remained high and therefore limited their applicability.

The current generation of inferior vena cava (IVC) filters began in 1967 with the introduction of the Mobin-Uddin umbrella 21 (FIG. 1C) which is described in further detail in U.S. Pat. No. 3,540,431. The Greenfield filter (FIG. 1D) was introduced in 1973 and is described in further detail in U.S. Pat. No. 3,952,747. These conical-shaped devices were placed endoluminaly in the IVC and utilized hooks or barbs 20, 30 to pierce the IVC wall and fix the position of the device. A variety of conical-shaped, percutaneously placed vena cava filters, based upon this concept are now available. For example, the TULIP with a filter structure 41 (FIG. 1E) further described in U.S. Pat. No. 5,133,733; the RECOVERY with a filter structure 51 (FIG. 1F) further described in U.S. Pat. No. 6,258,026; and the TRAPESE with a filter structure 61 (FIG. 1G) further described in U.S. Pat. No. 6,443,972.

The next advancement in filters added the element of recoverability. Retrievable filters were designed to allow removal from the patient subsequent to initial placement. Retrievable filters are generally effective at preventing PE yet they have a number of shortcomings, such as, for example: failure of the device to deploy into the vessel properly, migration, perforation of the vessel wall, support structure fracture, retrievability actually limited to specific circumstances, and formation of thrombosis on or about the device.

Problems associated with retrievable, conical-shaped devices, such as those illustrated in FIG. 1D, FIG. 1E and FIG. 1F, have been reported in the medical literature. These reported problems include tilting which makes it difficult to recapture the device and compromises filtration capacity. Hooks 30, 40, 50, 60 used to secure these devices have been reported to perforate the vessel wall, cause delivery complications, and fracture. A partially retrievable system is described in detail in pending U.S. Pat. No. 2004/0186512 (FIG. 1H). In this system, the filter portion 71 can be removed from the support structure 70, but the support structure remains in-vivo. All of these described devices share the common limitation that they can be retrieved from only one end. Each of the above referenced articles, patents and patent application are incorporated herein in its entirety.

In view of the many shortcomings and challenges that remain in the field of endoluminal filtering, there remains a need for improved retrievable, endoluminal filters.

### SUMMARY OF THE DISCLOSURE

In one aspect of the invention, there is provided an endoluminal filter comprising a first support member having a first end and a second end and a second support member attached to the first end of the first support member or the second end of the first support member and forming a crossover with the first support member. The endoluminal filter also comprises a material capture structure extending between the first and second support members, the crossover and the first end or the second end of the first support member. The endoluminal filter also comprises at least one tissue anchor on the first support member or the second support member. A radiopaque material is disposed on at least a portion of the material capture structure. In some embodiments, the material capture structure comprises a plurality of filaments and the radiopaque material is positioned at an intersection of two or more filaments. The radiopaque material may comprise a marker bead, capable of eluting drug (heparin, tpa, etc). In some embodiments, the marker bead comprises at least one lumen through which at least one filament is configured to be inserted. The marker bead may comprise two lumens. At least a portion of a surface of at least one lumen may comprise a material configured to be bonded to the at least one filament. In some embodiments, the marker bead comprises at least one protrusion positioned at an opening of the at least one lumen and through which the at least one filament is configured to be inserted. At least a portion of the protrusion may be configured to be bonded to the at least one filament. In some embodiments, an interior surface of the protrusion includes ridges and is configured to be swaged to the at least one filament. The material capture structure may comprise a plurality of filaments, and at least a portion of one of the plurality of filaments may be doped with a radiopaque material. In some embodiments, the radiopaque material comprises at least one of tungsten, tantalum, gold, barium, bismuth, palladium, and platinum. The endoluminal filter may further comprise an echogenic marker disposed on at least a portion of the material capture structure.

In another aspect, a method of positioning a filter device within a lumen is provided. The method comprises advancing a sheath containing the filter device through the lumen. The method also comprises deploying a portion of the filter device from the sheath into the lumen. The method comprises visualizing the material capture structure under fluoroscopy. The method also comprises deploying the material capture structure from the sheath into a position across the lumen. The method may also comprise recapturing at least a portion of the filter device within the sheath and re-deploying the at least a portion of the filter device into a position across the lumen. In some embodiments, the method comprises recapturing at least a portion of the material capture structure within the sheath and re-deploying the at least a portion of the material capture structure into a position across the lumen.

In yet another aspect, a method of retrieving a filter device from a body lumen is provided. The method comprises visualizing the material capture structure under fluoroscopy. The method also comprises advancing a sheath containing a snare proximate to a retrieval feature on the filter device. The method comprises exposing the snare and engaging the snare with the retrieval feature. In some embodiments, the method also comprises advancing the sheath over the filter device while maintaining positive control of the snare as the sheath. The method may also comprise pulling the snare and the filter device into the sheath. In some embodiments, the method comprises determining a position of the material capture structure, using fluoroscopy, prior to advancing the sheath containing the snare proximate to the retrieval feature on the filter device. The method may comprise using the position of the material capture structure to determine an approach path for advancing the sheath containing the snare proximate to the retrieval feature on the filter device.

In some embodiments, an endoluminal filter is provided. The filter comprises a first support member having a first end and a second end; a second support member attached to the first end of the first support member or the second end of the first support member and forming a crossover with the first support member; a material capture structure extending between the first and second support members, the crossover and the first end or the second end of the first support member, the material captures structure comprising a sheet of material comprising cutouts; at least one attachment positioned on a perimeter of the material capture structure, attachment configured to join the material capture structure to the first support member and the second support member; and at least one tissue anchor on the first support member or the second support member.

In another aspect, a method of manufacturing an endoluminal filter is provided. The method comprises providing a first support member having a first end and a second end; providing a second support member; attaching the second support member to the first end or second end of the first support member; forming a crossover with the first support member and the second support member; laser cutting a material to form a material capture structure; providing an attachment on a perimeter of the material capture structure; and attaching the material capture structure to the first support member and the second support member.

In some embodiments, an endoluminal filter is provided. The filter comprises a material capture structure formed using an additive manufacturing technique having a filter cell shape and size for use in filtering particulates no more than 3 mm in any dimension; a filter support frame sized for use within the vasculature of a mammal; and a filter securing element on the material capture structure adapted and configured to be coupled to the filter support frame so that in use the material capture structure extends across an opened support frame.

The filter can have a filter cell shape and size for use in filtering particulates no more than 1 mm in any dimension. In some embodiments, the filter has a filter cell shape and size for use in filtering particulates no more than 200 microns, 0.1 mm, 1 mm, 2 mm, 3 mm, 4 mm, 5 mm, 6 mm, 7 mm, 8 mm in any dimension. The filter securing element can be integrally formed as a part of the material capture structure. In some embodiments, the filter securing element further comprises a filter structure attachment, a frame attachment and a spacer joining the filter structure attachment to the frame attachment. The filter securing element can be formed using an additive manufacturing technique as separate element or integral to the material capture structure. The material capture structure can be one of a printed pattern or a woven pattern.

In another aspect, an endoluminal filter is provided. The filter comprises a first support member having a first end and a second end; a second support member attached to the first end of the first support member or the second end of the first support member and forming a crossover with the first support member; a material capture structure extending between the first and second support members, the crossover and the first end or the second end of the first support member, the material captures structure formed using an additive manufacturing technique; and at least one attachment positioned on a perimeter of the material capture structure, attachment configured to join the material capture structure to the first support member and the second support member. The attachment can be configured to be tied to the first support member or the second support member. The attachment can comprise a tab configured to fold over the first support member or second support member.

In some embodiments, a method of manufacturing an endoluminal filter is provided. The method comprises providing a first support member having a first end and a second end; providing a second support member; attaching the second support member to the first end or second end of the first support member; forming a crossover with the first support member and the second support member; using an additive manufacturing technique to form a material capture structure; providing an attachment on a perimeter of the material capture structure; and attaching the material capture structure to the first support member and the second support member.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1H illustrate various prior art filters;

FIGS. 2A-2C illustrate the response of a filtering device to changes in lumen size;

FIGS. 3, 3A-3B, 4, and 5 illustrate the interaction of a structural member with a lumen wall;

FIGS. 6A-6C, 7A-7G, and 8A-8D illustrate various aspects of the structural members in a filtering device

FIGS. 9A and 9B illustrate various aspects of a generally planer support frame;

FIGS. 10A and 10B illustrate various aspects of a non-planer support frame;

FIGS. 11, 12A-12B, and 13A-13C illustrate various aspects of and configurations for material capture structures;

FIGS. 14 and 14A-14C illustrate various aspects of a filtering device having three support frames;

FIG. 15 illustrates planes of symmetry for filtering devices;

FIGS. 16A and 16B illustrate the response of a filtering device when contacted by debris flowing in a lumen;

FIGS. 17-19 illustrate alternative filtering device aspects having different sized support frames and structural member lengths;

FIGS. 20-24 illustrate various alternative filtering device ends and structural member joining techniques;

FIGS. 25-26, and 27A-27C illustrate various alternative retrieval features;

FIGS. 28A-28C illustrate various techniques of joining or forming retrieval features;

FIG. 29 illustrates a filtering device with a retrieval feature positioned within a lumen.

FIGS. 30-33, 34A-34C, 35-37, 38A-38B, 39-50, 51A-51B, 52A-52D, and 53A-53D illustrate several alternatives techniques for joining material capture structures to support frames and forming filtering structures;

FIGS. 54A-54C, 55A-55E, 56, 57A-57B, 58, 59A-59G, 60A-60E, 61A-61C, 62, 63A-63B, 64A-64B, and 65A-65F illustrate several alternative filtering structures;

FIGS. 66 and 67 illustrate various filtering device configurations;

FIGS. 68A-68B, 69A-69D, 70, 71A-71B, 72A-72F, 73A-73D, 74A-74D illustrate various techniques related to the delivery, recovery and repositioning of filtering devices;

FIGS. 75A-75I, 76A-76E, 77A-77E, and 78A-78F illustrate several exemplary methods of using a filtering device;

FIGS. 79-82 illustrate several alternative filtering device configurations adapted for the delivery of pharmacological agents; and

FIGS. 83A-83E, 84A-84E, 85A-85D, 86A-86D, and 87 illustrate several filtering device prototypes.

FIG. 88 is a perspective view of an endoluminal filter having three tissue anchors;

FIGS. 89A and 89B illustrate individual filter components that may be assembled into the final version illustrated in FIG. 89C;

FIG. 89C is a perspective view of a final assembled filter;

FIGS. 90A and 90B illustrate proximal and distal filter ends with the tips of the elongated members modified to form fixation elements;

FIG. 90C is a perspective view of a filter assembly using the proximal distal end of FIGS. 90A and 90B;

FIG. 91 is a perspective view of a filter device performed by joining the device illustrated in FIG. 90A with the device illustrated in FIG. 90B;

FIG. 92 illustrates a fixation element formed in the end of an elongate body;

FIGS. 93A and 93B are perspective and cross section views respectively of a prior art fixation element having a transition section and a reduced diameter section;

FIG. 94 illustrates an embodiment of a filter structure proximal end formed from a single wire;

FIG. 95 illustrates an embodiment of a filter structure proximal end formed from a single wire with fixation elements from FIG. 104A;

FIGS. 96 and 97 illustrate filter devices with fixation elements in use within a lumen with the filtering structure in a upstream (FIG. 96) and downstream (FIG. 97) positions;

FIG. 98 illustrates a fixation element engaged with the side wall of lumen;

FIG. 99 illustrates a support frame without a material capture structure showing the placement and orientation of various fixation elements;

FIG. 100 illustrates the placement of the fixation elements about mid-distance between the ends and the crossover;

FIG. 101 illustrates the placement of fixation elements similar to FIG. 100 with additional of fixation elements positioned near the crossover and the ends;

FIG. 102 illustrates more than one fixation element positioned at the same location along the filtering device;

FIGS. 103A, 103B and 103C illustrate positioning of a fixation element on the elongate body (FIG. 103A) or to one side of the elongate body (FIGS. 103B and 103C);

FIGS. 104A and 104B illustrate a double ended fixation element (FIG. 104A) and attachment of a double ended fixation element to an elongate body (FIG. 104B);

FIG. 104C illustrates a double ended fixation element with different tip orientations attached to an elongate body;

FIGS. 105 and 106 illustrate tissue anchor embodiments having an end raised above the support member;

FIG. 107A illustrates a tissue anchor attached to a tube that is attached to a support member;

FIG. 107B illustrates a plurality of the tissue anchors illustrated in FIG. 107A positioned along a pair of support structures;

FIG. 108 illustrates tissue anchors formed in a tube that is placed over an elongate body or other portion of a filtering device;

FIG. 109 illustrates tissue anchors formed by cutting into an elongate body;

FIG. 110 illustrates a perspective view of tube with a surface modified to provide tissue engagement features;

FIGS. 111A and 111B illustrate alternative fixation features that may be mounted on, in or through the wall of a tube;

FIG. 112 is a perspective view of a tube based fixation element having a raised spiral form;

FIG. 113 illustrates a perspective view of one end of a filtering device where the retrieval feature includes a tissue engagement feature;

FIG. 114 illustrates a perspective view of one end of a filtering device where the retrieval feature terminates within the securing or attachment feature;

FIG. 115 illustrates a perspective view of one end of a filtering device where the retrieval feature terminates within the securing or attachment feature and the end of an elongate support structure is formed into a tissue engagement element;

FIG. 116A illustrates a perspective view of one end of a filtering device where the ends of elongate bodies pass through the securing or attachment feature and are formed into a retrieval feature and a tissue engagement element;

FIG. 116B is a section view through the securing or attachment feature shown in FIG. 116A;

FIG. 116C is a section view through the securing or attachment feature of FIG. 16A where separate tissue engagement and retrieval features are provided rather than formed in the ends of the elongate support structures;

FIGS. 117A and 117B illustrate perspective and bottom up views respectively of one end of a filtering device where the end of one elongate body pass through the securing or attachment feature and is formed into a retrieval feature and a tissue engagement element is formed in a portion of the securing or attachment feature;

FIG. 118 is a perspective view of a separate tissue engagement feature that is joined to a filtering device using the securing or attachment feature;

FIG. 119 illustrates an alternative embodiment of the tissue engagement element of FIG. 98 with the addition of a hollowed tip portion;

FIG. 120 illustrates an alternative embodiment of the tissue engagement element of FIGS. 93A and 93B with the addition of a hollowed tip portion;

FIGS. 121 and 122 illustrate an alternative embodiments of the tissue engagement elements of FIGS. 111A and 111B with the addition of a hollowed tip portion;

FIGS. 123A and 123B illustrate a perspective view of a filter device within a lumen and positioned for deployment where the filter device is stowed in a deployment sheath (FIG. 123A). The filter device is shown in phantom in the view illustrated in FIG. 123B;

FIGS. 124A-124E illustrate an exemplary positioning and filter deployment sequence;

FIGS. 125A-C illustrate one approach and recovery sequence for retrieving a deployed filtering device;

FIGS. 126A-D illustrate one approach and recovery sequence for retrieving a deployed filtering device.

FIG. 127 illustrates a perspective view of an embodiment of a filter device including fluorogenic markers.

FIG. 128 illustrates an embodiment of a filter cell including fluorogenic markers.

FIGS. 129A-D illustrate an exemplary method for joining a fluorogenic markers to a filter device.

FIG. 130 illustrates an alternative embodiment for joining a fluorogenic marker to a filter device.

FIGS. 131A-B illustrate perspective and cross-sectional views of a marker bead including protrusions.

FIGS. 132A-B illustrate perspective and cross-sectional views of an alternative embodiment of a marker bead including protrusions.

FIGS. 133A-D illustrate embodiments of doped filaments.

FIG. 134 illustrates an embodiment of a material capture structure including fluorogenic and echogenic markers.

FIG. 135 illustrates an embodiment of a laser cut material capture structure.

FIG. 136 illustrates an embodiments of a material capture structure attaching structure.

FIG. 137 illustrates an embodiment of the material capture structure of FIG. 135 attached to a filter frame.

FIG. 138 is a perspective view of a dual loop filter support frame having a material capture structure within and secured to the frame using one or more techniques illustrated in FIGS. 139A through 139E.

FIG. 139A illustrates an enlarged view of a material capture structure formed from a 3D printed weave with a portion of the perimeter shown attached to the support frame.

FIG. 139B illustrates an enlarged view of a material capture structure having an 3D printed securing structure formed along a portion of the material capture structure perimeter and shown attached to the support frame.

FIG. 139C illustrates an enlarged view of a material capture structure printed using a form of 3D printing with a portion of the perimeter shown attached to the support frame.

FIGS. 139B1-139B3 illustrate alternative embodiments of material capture structure securing elements having variable spacer configurations such as a discrete spacer (FIG. 139B1); no spacer (FIG. 139B2) and web spacer (FIG. 139B3).

FIG. 139D illustrates an enlarged view of a material capture structure having a number of separate attachments along the material capture structure perimeter. In this embodiment, each attachment is shown tied about and secured to the support frame. In this exemplary embodiment, each attachment is used to form a girth hitch or cow hitch about the support frame.

FIG. 139E illustrates an enlarged view of a material capture structure formed with a series of securing tabs along a portion of the perimeter. In this illustrative embodiment, two securing tabs are shown folded over the support frame and then attached back onto itself.

FIG. 140 is a perspective view of an illustrative material capture structure secured to a double loop filter support frame using one or more of the techniques illustrated and described in FIGS. 139A-139E.

FIG. 141 is a perspective view of an illustrative material capture structure secured to a double loop filter support frame using one or more of the techniques illustrated and described in FIGS. 139A-139E.

FIG. 142 is a perspective view of an illustrative material capture structure secured to a double loop filter support frame using one or more of the techniques illustrated and described in FIGS. 139A-139E.

FIG. 143 is a perspective view of an illustrative material capture structure secured to a double loop filter support frame using one or more of the techniques illustrated and described in FIGS. 139A-139E.

FIG. 144 is a perspective view of an illustrative material capture structure secured to a double loop filter support frame using one or more of the techniques illustrated and described in FIGS. 139A-139E.

FIG. 145 is a perspective view of an illustrative material capture structure secured to a double loop filter support frame using one or more of the techniques illustrated and described in FIGS. 139A-139E.

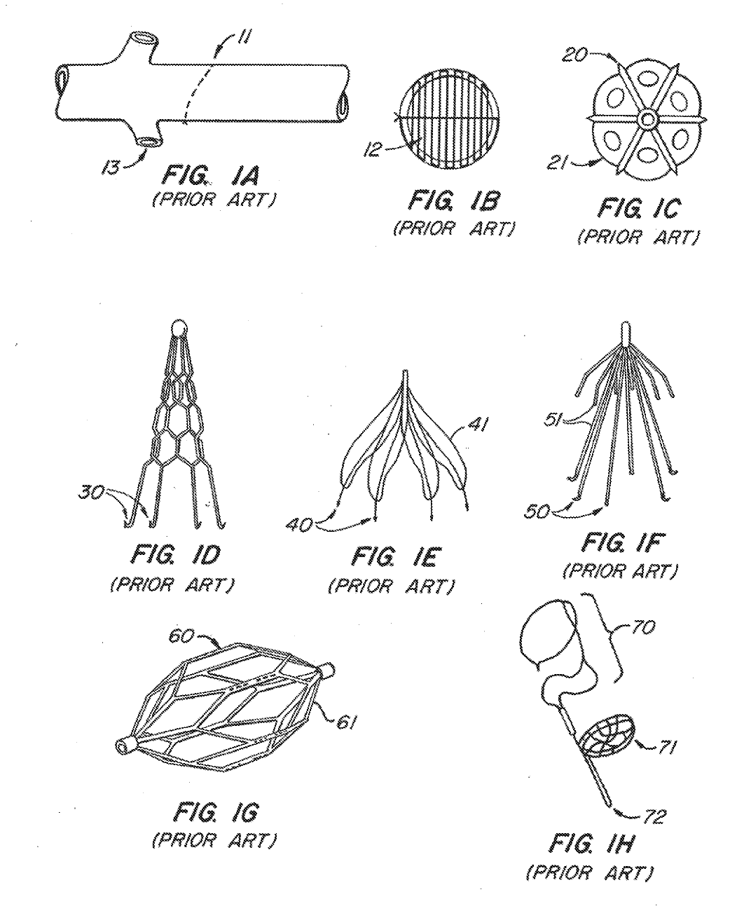

### DETAILED DESCRIPTION

There remains a clinical need for improved endoluminal filter devices and methods. Improved endoluminal filter devices provide effective filtration over a range of lumen sizes and are easy to deploy into and retrieve from a lumen. In addition, improved endoluminal filter devices minimize thrombosis formation or tissue ingrowth on the device and are resistant to migration along the lumen. Embodiments of the filter devices of the present invention provide many and in some cases all of the features of improved endoluminal filters and have a number of uses such but are not limited to: embolic protection, thrombectomy, vessel occlusion, and tethered or untethered distal protection.

Several embodiments of the present invention provide improved filtration devices that are durable, provide effective and nearly constant filter capacity over a range of lumen sizes and are easily delivered and removed from a lumen via either end of the device. Additionally, embodiments of the present invention can be delivered into and retrieved from a lumen using minimally invasive surgical techniques. One aspect of an embodiment of the present invention is the construction of support structure elements using a shape memory material. The shape memory material may have a pre-shaped form that ensures the support elements are uniformly collapsible and, when deployed, provides a pre-defined range of controllable force against the lumen wall without use of hooks or barbs. Alternatively, hooks barbs, or other fixation elements or devices may be used in conjunction with an embodiment of a filtering device as described below.

The elongate support structure elements are configured to collapse and expand with natural vessel movements while maintaining constant apposition with the vessel wall. One result is that the support structure shape and size track to vessel movements. As a result, the filter density and capacity of embodiments of the present invention remain relatively independent of changes in vessel size. Moreover, the self centering aspect of the support structure ensures the filtration device provides uniform filtration across the vessel diameter. As such, embodiments of the present invention provide generally constant filtration capacity that is maintained across the entire vessel lumen and during vessel contractions and expansions.

Uniform filter capacity is a significant improvement over conventional devices. Conventional devices typically have a filter capacity that varies radially across a lumen. The radial variation in filter capacity usually results from the fact that conventional filtration elements have a generally wider spacing at the periphery of the lumen and closer spacing along the central lumen axis. The result is that larger emboli can escape along the lumen periphery. During vessel expansions and contractions, the radial variations in filter capacity are exacerbated in conventional devices.

Another advantage of some embodiments of the present invention is that when released from a constrained state (i.e., within a delivery sheath), the device assumes a pre-determined form with elongate support members that extend along and self center the device in the vessel. These elongate support members exert atraumatic radial force against the vessel wall to prevent or minimize device migration. In some embodiments, radial forces generated by the elongate support members work in cooperation with hooks, barbs or other fixation devices to secure the device within the vessel. Hooks, barbs or other fixation devices or elements may be used as an added precaution against migration of the filtering device while in a lumen. When device retrieval is initiated, the uniformly collapsible form of the elongate support members causes the elongate support members to pull away from the vessel wall as the device is being re-sheathed. The movement of the elongate members away from the vessel wall facilitates the atraumatic removal of the device from the vessel wall. Additionally, in those embodiments having hooks, barbs or other fixation devices or elements, elongate member movement during retrieval also facilitates withdrawal of the fixation elements from the lumen wall.

Additional embodiments of the present invention may include a retrieval feature on one or both ends of the device. The use of retrieval features on both ends of the device allows deployment, repositioning and removal of the device to be accomplished from either end of the device. As a result, the use of retrieval features on both ends of the device enables both antegrade or retrograde approaches to be used with a single device. The retrieval feature may be integral to another structural member or a separate component. In some embodiments, the retrieval feature is collapsible and may have a curved shape or a generally sinusoidal shape. Additional aspects of retrieval features are described below.

### General Principals and Construction

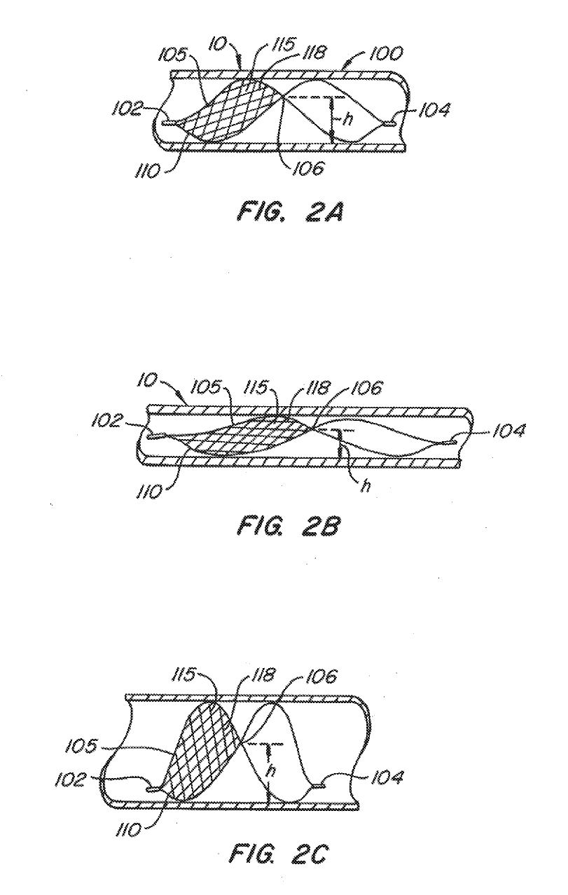

FIG. 2A illustrates an embodiment of a filtering device 100 of the present invention positioned within a lumen 10. The lumen 10 is cut away to show the position of filter 100 deployed into within a lumen and in contact with the lumen wall. The filter 100 includes a first elongate member 105 and a second elongate member 110. The elongate members are joined to form ends 102, 104. The elongate members cross but are not joined to one another at crossover 106. In one embodiment, the elongate members have first and second sections. First sections extend between the end 102 and the crossover 106 and the second sections extend from the crossover 106 to the second end 104. While some embodiments contact the lumen in different ways, the illustrated embodiment has the ends 102, 104 against one side of the lumen interior wall while the crossover 106 contacts the other side of the lumen interior wall with the elongate bodies in constant or nearly constant apposition along the lumen interior wall between the ends 102, 104.

Material (i.e., thrombus, plaque and the like) flowing through the lumen 10 of a size larger than the filtering size of the material capture structure 115 is captured between or cut down by the filaments 118. In the illustrated embodiment of FIG. 2A, the material capture structure 115 is supported by a rounded frame formed by the elongate members 105, 110 formed between the end 102 and the crossover 106. Another rounded frame formed between the crossover 106 and the second end 104 and could also be used to support a material capture structure of the same or different construction and filter capacity of the a material capture structure 115. As such, a material removal structure supported by one rounded frame may be configured to remove material of a first size and the material removal structure supported by the other rounded frame may be configured to remove material of a second size. In one embodiment, the material removal structure in the upstream rounded frame removes larger size debris than material removal structure in the downstream rounded frame. Also illustrated in FIGS. 2A-2C is how the filter cells 119 that make up the material capture structure is 115 maintain their size and shape relatively independent of movement of the first and second structural members 105, 110 over a physiological range of vessel diameters.

FIGS. 2B and 2C illustrate how the elongate support structure elements of embodiments of the present invention are configured to collapse and expand with natural vessel movements while maintaining constant apposition with the vessel wall. FIGS. 2A, 2B and 2C also illustrate how devices according to embodiments of the present invention are both radially and axially elastic. In response to vessel size changes, ends 102, 104 move out as the vessel size decreases (FIG. 2B) and then move in as the vessel size increases (FIG. 2C). In addition, the device height “h” (measured from the lumen wall in contact with ends 102, 104 to crossover) also changes. Device height “h” changes in direct relation to changes in vessel diameter (i.e., vessel diameter increases will increase device height “h”). As such, device height (“h”) in FIG. 2C is greater than device height (“h”) in FIG. 2A which is in turn greater than the device height (“h”) in FIG. 2B.

FIGS. 2A, 2B and 2C also illustrate how a single sized device can be used to accommodate three different lumen diameters. FIG. 2C illustrates a large lumen, FIG. 2A a medium sized lumen and FIG. 2B a small sized lumen. As these figures make clear, one device can adapt to cover a range of vessel sizes. It is believed that only 3 device sizes are needed to cover the range of human vena cava interior diameters that range from approximately 12-30 mm with an average interior diameter of 20 mm. Also illustrated is the static or nearly static filter capacity of the material capture structure 115. In each different vessel size, the material capture structure 115, the filaments 118 and filter cell 119 maintain the same or nearly the same shape and orientation within the support frame formed by the elongate bodies. These figures also illustrate the dynamic shape changing aspect of the device that may also be used to accommodate and conform to vessel irregularities, tortuosity, flares and tapers and while remaining in apposition to the wall. Because each elongate body may move with a high degree of independence with respect to the other, the loops or support frames formed by the elongate bodies can also independently match the shape/diameter of the lumen section in which it is placed.

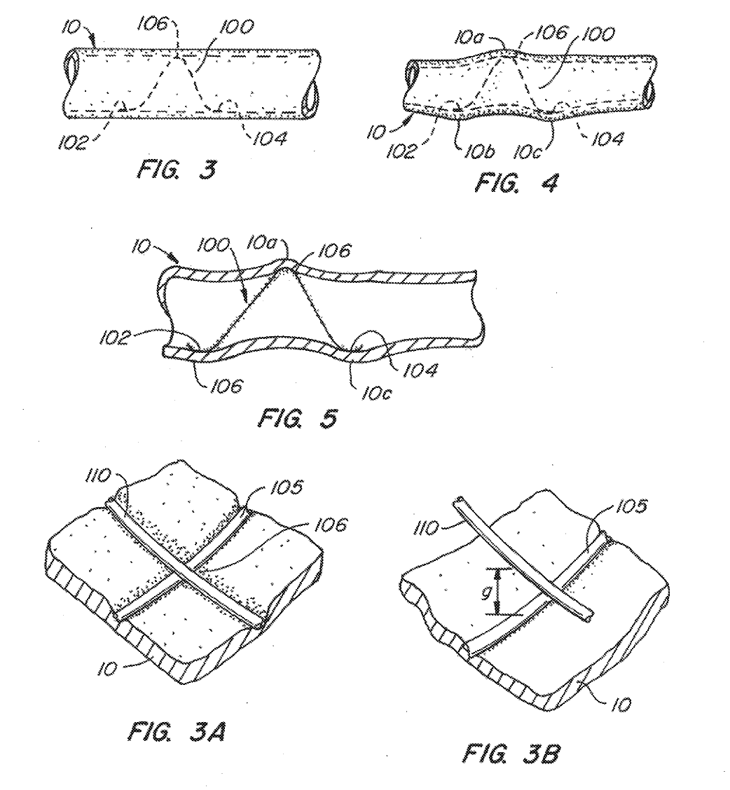

FIGS. 3, 3A and 3B illustrate the device 100 deployed into the lumen 10. As illustrated in FIG. 3, the device 100 is oriented in the lumen with the ends 102, 104 along one side of the interior vessel wall with the crossover 106 on the opposite side. FIG. 3 illustrates an embodiment of a device of the present invention that is shaped to fit within the lumen 10 without distending the lumen. In FIG. 3A the elongate bodies 105, 110 are in contact but are not joined at crossover 106. In FIG. 3B the elongate bodies 105, 110 cross one another at crossover 106 but are separated (i.e., by a gap “g”).

FIGS. 4 and 5 illustrate how aspects of the device design can be modified to increase the radial force applied against the interior wall of lumen 10. Devices having increased fixation force may be useful for some applications, such as vessel occlusion or for distal protection when a large amount of debris is expected. If a device is not intended to be retrieved (i.e., permanently installed into a lumen) then high radial force design devices may be used to ensure the device remains in place and distention may be used to trigger a systemic response (i.e., a tissue growth response) in the lumen to ensure device ingrowth and incorporation with the lumen interior wall.

Filter device embodiments of the present invention having low or atraumatic radial force are particularly useful in retrievable devices. As used herein, atraumatic radial force refer to radial forces produced by a filtering device embodiment that meets one or more of the following: radial forces high enough to hold the device in place with little or no migration and without damaging or overly distending the lumen interior wall; radial forces high enough to hold the device in place but while triggering little or no systemic response for the vessel wall; or forces generated by device operation that trigger reduced systemic response or a systemic response below that of a conventional filter.

In contrast to the device sized in FIG. 3 to minimize vessel distention, FIG. 4 illustrates a device 100 configured to exert greater radial force to a degree to cause lumen wall to distend. FIGS. 4 and 5 illustrate lumen wall distention by the end 102 (distention 10b), by the crossover 106 (distention 10a), and by the end 104 (distention 10c). Although not shown in these figures, the elongate bodies would likely distend the lumen along their length as well.

The radial force of a device may be increased using a number of design factors. Radial force may be increased by increasing the rigidity of the elongate body by, for example, using an elongate body with a larger diameter. Radial force may also be increased when forming the shapes of the elongate bodies (i.e., during the heat treat/set processes for Nitinol devices and the like), as well as in the material composition and configuration.

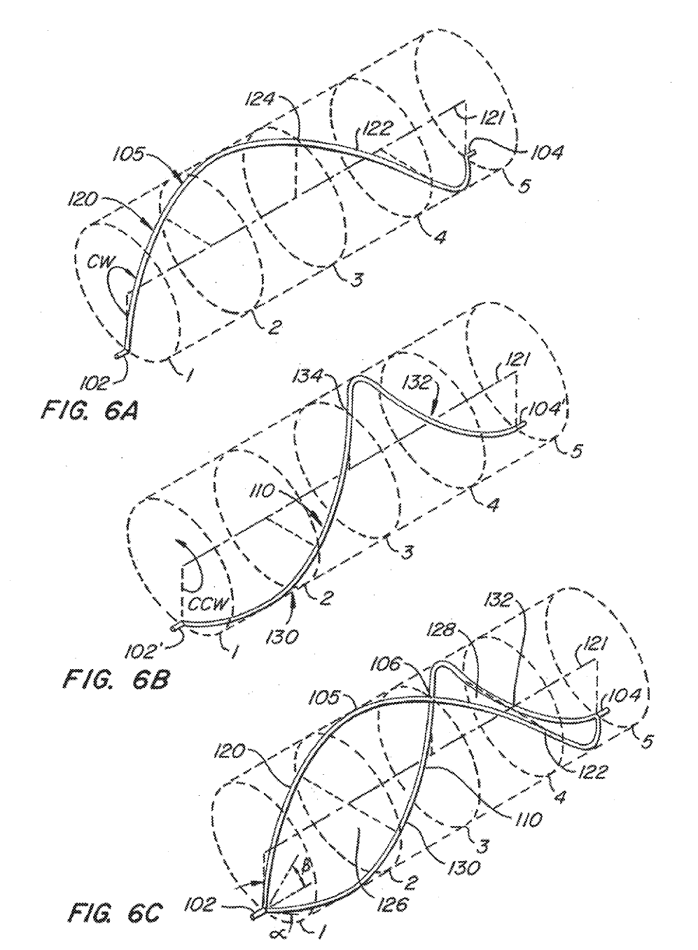

Additional details of an embodiment of the support members 105, 110 may be appreciated with reference to FIGS. 6A, 6B and 6C. FIGS. 6A, 6B illustrate the support members separately and then assembled together (FIG. 6C) about device axis 121. In general, the device axis 121 is the same as the axis along the central of a lumen into which the device is deployed. For purposes of illustration, the support members 105, 110 will be described with reference to a sectioned lumen shown in phantom having a generally cylindrical shape. The support members may also be thought of as deployed within and/or extending along the surface of an imaginary cylinder.

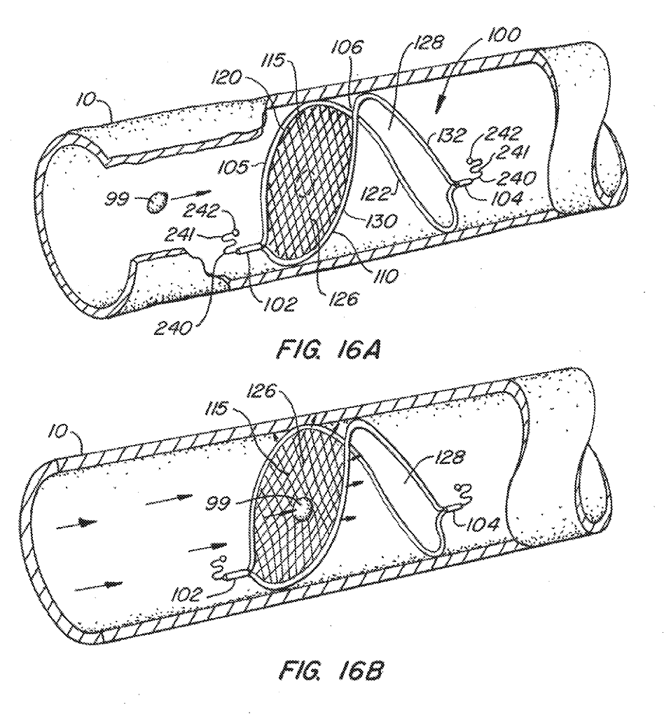

In the illustrative embodiments of FIGS. 6A, 6B and 6C, the support members 105, 110 are shown in an expanded, pre-defined shape. In one embodiment, the support members are formed from MRI compatible materials. The support members contain no sharp bends or angles to produce stress risers that may lead to fatigue issues, vessel erosion, and facilitate device collapse. In some embodiments, each elongate member is conventionally formed by constraining a shape memory material such as a shape memory metal alloy or shape memory polymer on a cylindrical shaping mandrel that contains pins to constrain the material into the desired shape. Thereafter, the material can be subjected to a suitable conventional heat treatment process to set the shape. One or more planes of symmetry (i.e., FIG. 15) may be provided, for example, by forming both elongate members on a single mandrel and at the same time. Other conventional processing techniques may also be used to produce symmetrical filtering device embodiments. Additionally, retrieval features described herein (if present) may be directly formed on the wire ends during support member processing. In addition, multiple devices, in a series on a long mandrel, can be made using these methods.

Examples of suitable shape memory alloy materials include, for example, copper-zinc-aluminium, copper-aluminum-nickel, and nickel-titanium (NiTi or Nitinol) alloys. Nitinol support structures have been used to construct a number of working prototypes of filter devices of the present invention as well as for use in ongoing animal studies and human implants. Shape memory polymers may also be used to form components of the filter device embodiments of the present invention. In general, one component, oligo(e-caprolactone) dimethacrylate, furnishes the crystallizable “switching” segment that determines both the temporary and permanent shape of the polymer. By varying the amount of the comonomer, n-butyl acrylate, in the polymer network, the cross-link density can be adjusted. In this way, the mechanical strength and transition temperature of the polymers can be tailored over a wide range. Additional details of shape memory polymers are described in U.S. Pat. No. 6,388,043 which is incorporated herein by reference in its entirety. In addition, shape memory polymers could be designed to degrade. Biodegradable shape memory polymers are described in U.S. Pat. No. 6,160,084 which is incorporated herein by reference in its entirety.

It is believed that biodegradable polymers may also be suited to form components of the filter device embodiments of the present invention. For example, polylactide (PLA), a biodegradable polymer, has been used in a number of medical device applications including, for example, tissue screws, tacks, and suture anchors, as well as systems for meniscus and cartilage repair. A range of synthetic biodegradable polymers are available, including, for example, polylactide (PLA), polyglycolide (PGA), poly(lactide-co-glycolide) (PLGA), poly(e-caprolactone), polydioxanone, polyanhydride, trimethylene carbonate, poly(β-hydroxybutyrate), poly(g-ethyl glutamate), poly(DTH iminocarbonate), poly(bisphenol A iminocarbonate), poly(ortho ester), polycyanoacrylate, and polyphosphazene. Additionally, a number of biodegradable polymers derived from natural sources are available such as modified polysaccharides (cellulose, chitin, dextran) or modified proteins (fibrin, casein). The most widely compounds in commercial applications include PGA and PLA, followed by PLGA, poly(e-caprolactone), polydioxanone, trimethylene carbonate, and polyanhydride.

While described as forming the support structures, it is to be appreciated that other portions of the filter device may also be formed from shape memory alloys, shape memory polymers or biodegradable polymers. Other filter device components that may also be formed from shape memory alloys, shape memory polymers or biodegradable polymers include, for example, all or a portion of a retrieval feature, a material capture structure or an attachment between a material capture structure and a support structure. Additionally or alternatively, the devices described herein may have all or a portion of their components formed from medical grade stainless steel.

FIG. 6A illustrates the first support member 105 extending from an end 102 to an end 104 along in a clockwise manner about the lumen interior wall (sectioned phantom lines) and the device axis 121. The support member 105 extends from the end 102 in section 1 at the 6 o'clock position, up to the 9 o'clock position in section 2, the 12 o'clock position in section 3, the 3 o'clock position in section 4 to the end 104 at the 6 o'clock position in section 5. The support member 105 has two sections 120, 122 on either side of an inflection point 124. The inflection point 124 is positioned at about the 12 o'clock position in section 3. The radius of curvature of the sections 120, 122 may be the same or different. The cross section shape of the support member 105 is generally circular but may have one or more different cross section shapes in alternative embodiments.

FIG. 6B illustrates the second support member 105 extending from an end 102′ to an end 104′ along in a counter-clockwise manner about the lumen interior wall (sectioned phantom lines) and the device axis 121. The support member 110 extends from the end 102′ in section 1 at the 6 o'clock position, up to the 3 o'clock position in section 2, the 12 o'clock position in section 3, 9 o'clock position in section 4 to the end 104′ at the 6 o'clock position in section 5. The support member 110 has two sections 130, 132 on either side of an inflection point 134. The inflection point 134 is positioned at about the 12 o'clock position in section 3. The radius of curvature of the sections 120, 122 may be the same or different. The cross section shape of the support member 105 is generally circular but may have one or more different cross section shapes in alternative embodiments.

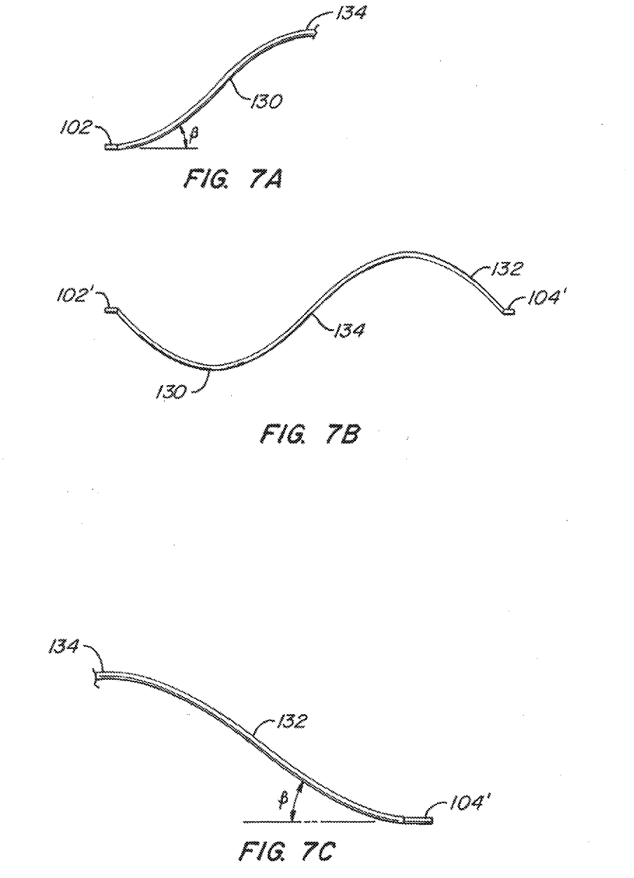

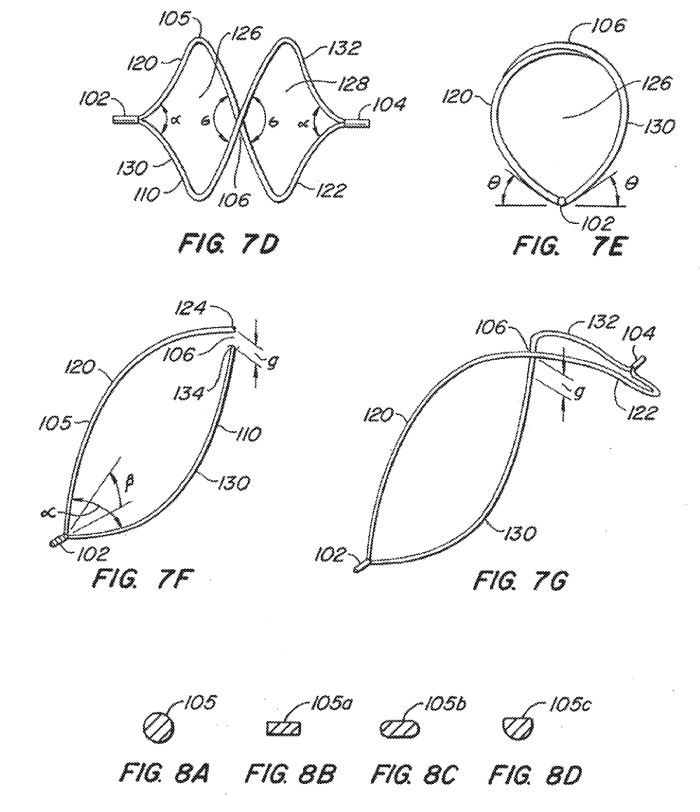

FIG. 6C illustrates the crossover 106 and first and second support members 105, 110 joined together at the ends. The first sections 120, 130 form a rounded frame 126. The angle β is formed by a portion of the lumen wall contacting end 102 and a plane containing the frame 126 and is referred to as the take off angle for the elongate members at end 102. In one alternative, the angle β is formed by a portion of the lumen wall contacting end 102 and a plane containing all or a portion of one or both sections 120, 130. In yet another alternative, the angle β is formed by a portion of the lumen wall contacting end 102 and a plane containing all or a portion of end 102 and all or a portion of the crossover 106. Another angle β is formed on end 104 as discussed above but in the context of end 104, a portion of the lumen wall contacting end 104, sections 122, 132 and the rounded frame 128 as illustrated in FIGS. 7A-7C. An angle formed by the support frames 126, 128 ranges generally between 20 degrees to 160 degrees in some embodiments and generally between 45 degrees to 120 degrees in some other embodiments.

FIG. 7A is a side view of section 130 in FIG. 6B, FIG. 7B is a top down view of FIG. 6B and FIG. 7C is side view of section 132 in FIG. 6B. The angle β ranges generally between 20 degrees to 160 degrees in some embodiments and generally between 45 degrees to 120 degrees in some other embodiments. The angle α is formed by a portion of section 120, a portion of section 130 and the end 102. Alternatively, the angle α is formed by the end 102 and tangents formed with a portion of the sections 120, 130. Another angle α is formed on end 104 as discussed above but in the context of end 104, a portion of the lumen wall contacting end 104 and sections 122, 132. The angle α ranges generally between 40 degrees to 170 degrees in some embodiments and generally between 70 degrees to 140 degrees in some other embodiments.

FIG. 7D illustrates a top down view of FIG. 6C. The angle α is defined as the angle between a portion of section 120 between the inflection point 124 and the end 102 on one side and a portion of section 130 between the inflection point 134 and the end 102′ on the other side. The angle α is also defined as the angle between a portion of section 122 between the inflection point 124 and the end 104 on one side and a portion of section 132 between the inflection point 134 and the end 104′ on the other side. The angle α defined by sections 120, 130 may be the same, larger, or smaller than the angle α formed by the sections 122, 132. The angle α ranges generally between 10 degrees to 180 degrees in some embodiments and generally between 45 degrees to 160 degrees in some other embodiments.

FIG. 7E illustrates an end view of FIG. 6C taken from end 102. The angle θ is defined as the angle between a plane tangent to a portion of section 120 and a plane containing the end 102 that is also generally parallel to the device axis 121. An angle θ may also be defined as the angle between a plane tangent to a portion of section 130 and a plane containing the end 102 that is also generally parallel to the device axis 121. The angle θ defined by section 120 may be the same, larger, or smaller than the angle θ formed by the section 130. Similarly, an angle θ may be defined as discussed above and using as the angle between a plane tangent to a portion of section 122 or 132 and a plane containing the end 102 that is also generally parallel to the device axis 121. The angle θ ranges generally between 5 degrees to 70 degrees in some embodiments and generally between 20 degrees to 55 degrees in some other embodiments.

FIGS. 7F and 7G are perspective views of an alternative embodiment of the device illustrated in FIG. 6C. In the embodiment illustrated in FIGS. 7F and 7G, the support member 110 crosses underneath and does not contact the support member 105 at the crossover 106. The gap “g” between the support members is also illustrated in the FIG. 7G.

FIG. 8A illustrates the elongate body 105 with a generally circular cross section. However, many other cross section shapes are possible and may be used such as, for example, rectangular elongate body 105a (FIG. 8B), rectangular elongate body with rounded edges (not shown), oval elongate body 105b (FIG. 8C) and circular elongate body with a flattened edge 105c (FIG. 8D). In some embodiments, an elongate body will have the same cross section along its length. In other embodiments, an elongate body will have different cross sections along its length. In another embodiment, an elongate body has a number of segments and each segment has a cross section shape. The segment cross section shapes may be the same or different. The cross section shape of the elongate member is a factor used to obtain the desired radial force along the elongate member. The material used to form the elongate body (i.e., a biocompatible metal alloy such as Nitinol) may be drawn to have a desired cross section shape, or drawn in one cross section shape and then treated using conventional techniques such as grinding, laser cutting and the like to obtain the cross section shape were desired.

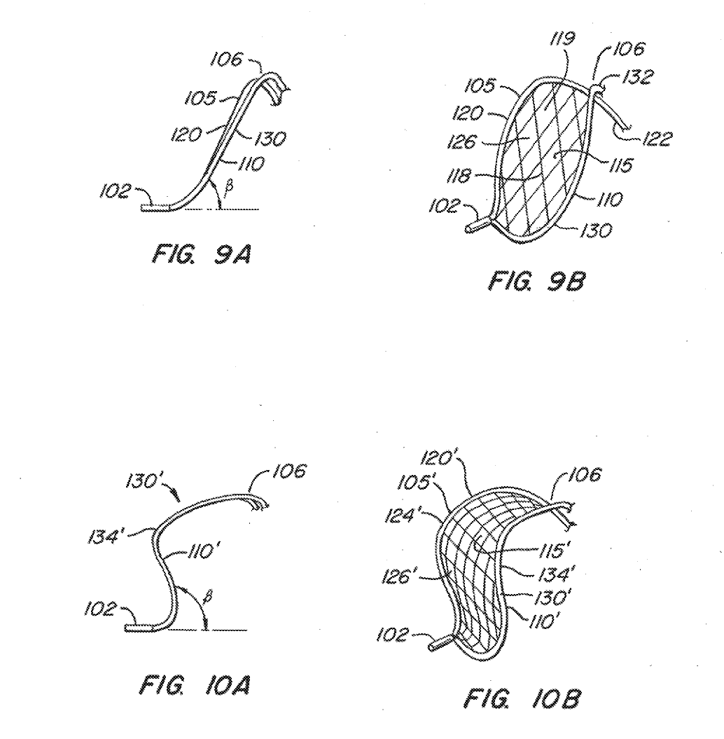

FIGS. 9A, 9B illustrate an embodiment of a material capture structure 115 extended across a generally planar, rounded frame 126 formed by the support members. FIG. 9A is a slight perspective view of a side view of the device. In this embodiment, sections 120, 130 of the support members lie mostly within in a single plane (i.e., in a side view of FIG. 9A section 110 is visible and blocks view of section 120) that also holds the rounded frame 126. FIG. 9B is a perspective view showing the material capture structure 115 extended between and attached to rounded frame 126. In this embodiment, the capture structure 115 extends across and is attached to the first sections 120, 130. In this embodiment, the material capture structure is a plurality of generally rectangular filter cells 119 formed by intersecting filaments 118. Other types of filter structures are described in greater detail below and may also be supported by the support frames formed by the structural members. In some embodiments such as FIGS. 9A and 9B, the angle β may also define the angle between the device axis and a plane containing a material capture structure.

The support frame 126 and the material capture structure 115 is not limited to planar configurations. Non-planar and compound configurations, for example, are also possible as illustrated in FIGS. 10A and 10B. FIG. 10A is a side view of a non-planar structural support 110′ having another inflection point 134′ between the inflection point 134 and the end 102. The structural support 110′ has more than one different radius of curvature between the end 102 and the crossover 106. In some embodiments, there could be more than one radius of curvature between the end 102 and the inflection point 134′ as well as be more than one radius of curvature between the inflection point 134′ and the inflection point 134. As a result, section 130′ is a section possibly having different shapes, a number of different curvatures and at least one inflection point. As seen in FIG. 10B, the support structure 105′ is also non-planar with more than one different radius of curvature between the end 102 and the inflection point 124. In some embodiments, there could be more than one radius of curvature between the end 102 and the inflection point 124′ as well as be more than one radius of curvature between the inflection point 124′ and the inflection point 124. As a result, section 120′ is a section having different shapes, a number of different curvatures and one or more inflection points. Similar non-planar configurations may be used on end 104. The material capture structure 115′ is adapted to conform to the shape of non-planar frame 126′ to produce a non-planar filter support structure.

FIG. 11 illustrates a material capture structure 115 that remains in a generally planar arrangement between opposing portions of the support members 105, 110. In addition to FIG. 10B above, other alternative non-planar capture structures are possible even if the support frame is generally planar. FIG. 12A is a perspective view of a non-planar capture structure 245 within a generally planar support frame formed by support members 105, 110. Capture structure 245 is formed by intersecting strands, fibers, filaments or other suitable elongate material 218 to form filter cells 219. The capture structure 245 is slightly larger than the support frame dimensions resulting in a filter structure that is deformed out of the plane formed by the support structure as illustrated in FIG. 12B.

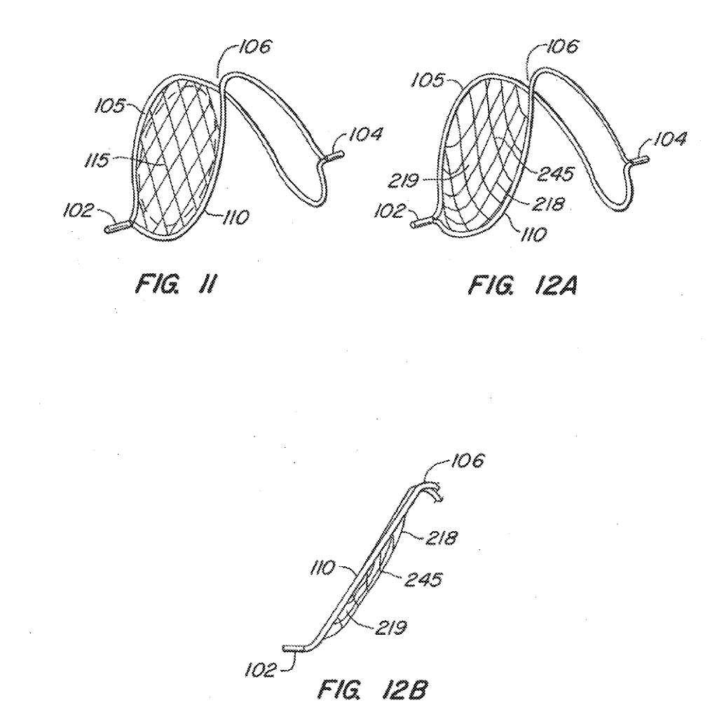

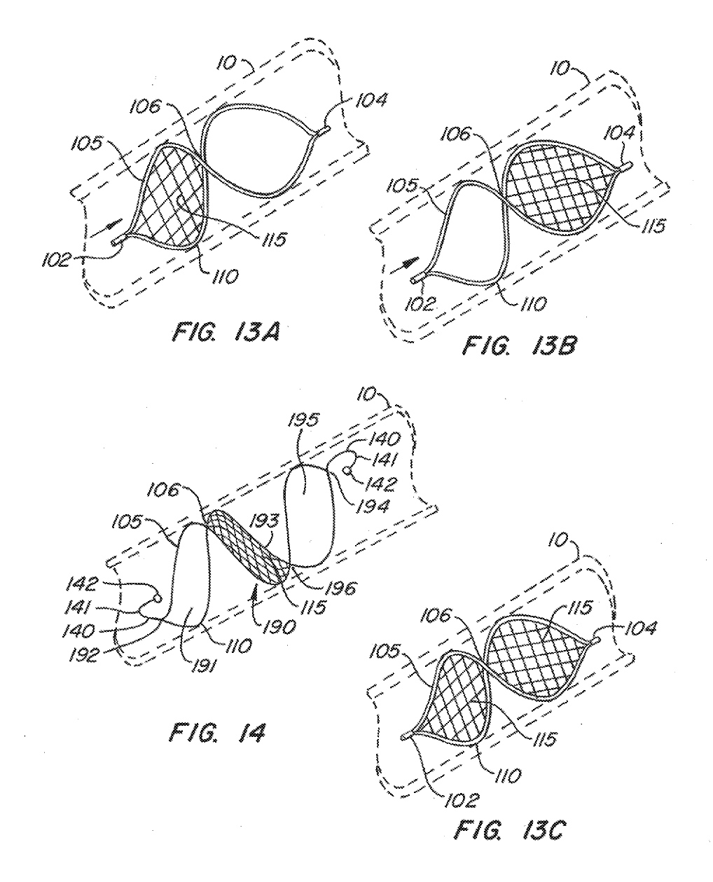

The material capture structure 115 may be in any of a number of different positions and orientations. FIG. 13A illustrates an embodiment of a filter of the present invention having two open loop support frames formed by support members 105, 110. Flow within the lumen 10 is indicated by the arrow. In this embodiment, the material capture structure 115 is placed in the upstream open loop support structure. In contrast, the material capture structure may be positioned in the downstream open loop support structure (FIG. 13B). In another alternative configuration, both the upstream and the downstream support frames contain material capture structures 115. FIG. 13C also illustrates an embodiment where a material capture structure is placed in every support loop in the device.

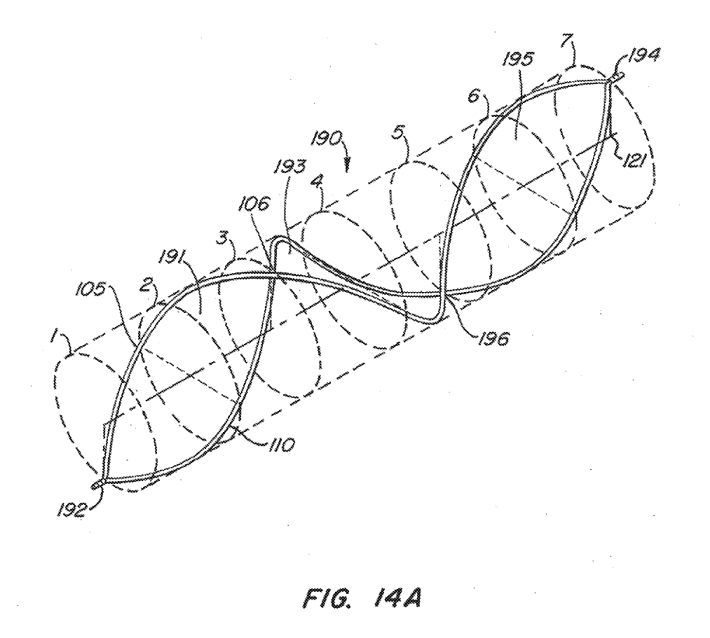

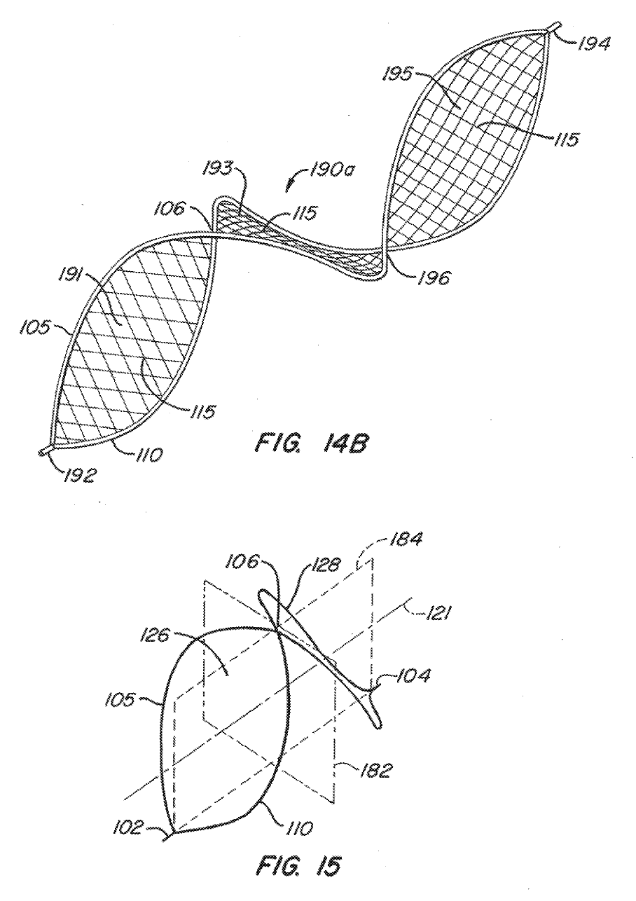

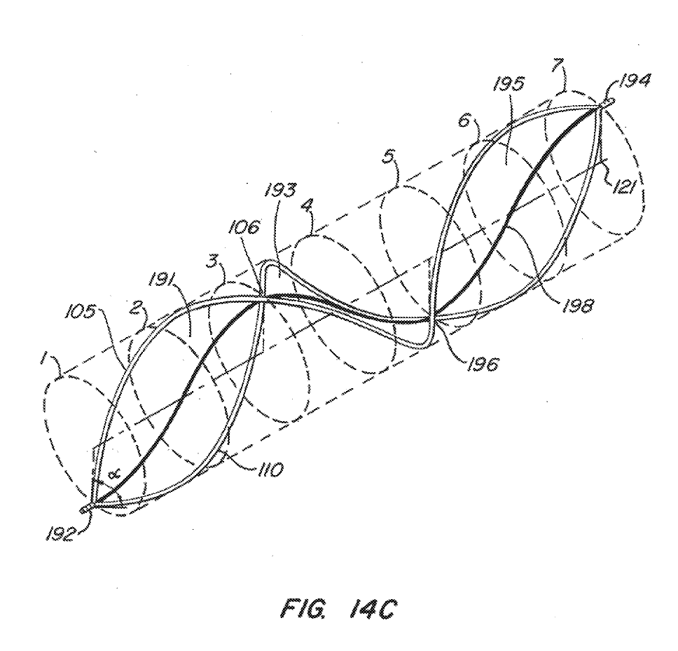

There are filter device embodiments having equal numbers of support frames with capture structures as support frames without capture structures (e.g., FIGS. 13A and 13B). There are other embodiments having more support frames without capture structures than there are support frames with capture structures. FIG. 14 illustrates a filter embodiment 190 having more support frames without capture structures than support frames with captures structures. The filter device 190 has two support members 105, 110 that are positioned adjacent to one another to form a plurality of support frames that are presented to the flow within the lumen 10. Alternatively, the plurality of support frames positioned to support a material capture structure across the flow axis of the device 190 or the lumen 10. The support members are joined together at end 192 and have two inflection points before being joined at end 194. The support members 105, 110 cross over one another at crossovers 106 and 196. The support frame 191 is between end 192 and crossover 106. The support frame 193 is between the crossovers 106, 196. The support frame 195 is between the cross over 196 and the end 194.

In addition, the filter device 190 has a retrieval feature 140 on each end. The retrieval feature 140 has a curved section 141 ending with an atraumatic tip or ball 142. The retrieval feature 140 rises up above the lumen wall placing the ball 142 and all or a portion of the curved section 141 into the lumen flow path to simplify the process of snaring the device 190 for retrieval or repositioning. Having a retrieval feature on each end of the device allows the device 190 to be recovered from the upstream or downstream approach to the device in the lumen 10. Various aspects of retrieval feature embodiments of the present invention are described in greater detail below.

FIG. 14A illustrates the filter 190 imposed on a phantom cylinder having 7 sections. The retrieval features 140 have been omitted for clarity. The first support member 105 extends clock wise from end 192 about and along the axis of the device 121. The first support member 105 crosses section 2 at the 9 o'clock position, section 3 and the crossover 106 at the 12 o'clock position, section 4 at the 3 o'clock position, section 5 and the crossover 196 at the 6 o'clock position, section 6 at the 9 o'clock position and section 7 and the end 194 at the 12 o'clock position. The second support member 110 crosses section 2 at the 3 o'clock position, section 3 and the crossover 106 at the 12 o'clock position, section 4 at the 9 o'clock position, section 5 and the crossover 196 at the 6 o'clock position, section 6 at the 3 o'clock position and section 7 and the end 194 at the 12 o'clock position. FIG. 14B illustrates an alternative device embodiment 190a that is similar to the device 190 except that all support frames formed by the elongate members is used to support a material capture structure. In the illustrated embodiment, frames 191, 193 and 195 each support at material capture structure 115.

FIG. 14C illustrates an alternative configuration of filter 190. The filter device 190b is similar to device 190 and 190a and includes an additional support member 198 extending along the support member 105. In one embodiment, the additional support member 198 extends along the device axis 121, is positioned between the first and the second support members 105, 110 and is attached to the first end 192 and the second end 194. In the illustrative embodiment, the third support member 198 begins at end 192 and the 6 o'clock position in section 1, crosses section 3 and the crossover 106 at the 12 o'clock position, crosses section 5 and the crossover 196 at the 6 o'clock position, and ends at the 12 o'clock position in section 7 at the end 194.

FIG. 15 illustrates the planes of symmetry found in some filter device embodiments of the present invention. The filtering structure that would be supported by one or both of the support frames is omitted for clarity. In one aspect, FIG. 15 illustrates an embodiment of an endoluminal filter of the present invention having a support structure that is generally symmetrical about a plane 182 that is orthogonal to the flow direction of the filter or filter axis 121 and contains a crossover point 106 between two structural elements of the support structure 105, 110. In another aspect, FIG. 15 illustrates an embodiment of an endoluminal filter of the present invention having a support structure that is generally symmetrical about a plane 184 that is parallel to the flow direction of the filter (i.e., axis 121) and contains both ends of the support structure 102, 104. It is to be appreciated that some filter device embodiments of the present invention may have either or both of the above described symmetrical attributes. It is to be appreciated that the above described symmetrical attributes are also applicable to the construction of embodiments of the material capture structures alone or as installed in a filter.

FIGS. 16A and 16B illustrate the response of a filter device 200 in response to a piece of clot material 99 contacting the material capture structure 115. The direction of flow and movement of the clot material 99 within lumen 10 is indicated by the arrows. The filter device 200 is similar to the embodiments described above with regard to FIGS. 6A-7G with the addition of the retrieval features 240 added to the ends 102, 104. The retrieval feature 240 has a curved section with multiple curves 141 that terminate with an atraumatic end 242. The multiple curves 141 are advantageously configured to collapse about a retrieval device (i.e., a snare in FIGS. 71A, 71B) to facilitate device 100 capture during retrieval. In this illustrative embodiment the multiple curves are generally shaped like a sinusoid and the end 242 is shaped like a ball or a rounded tip.

It is believed that upon embolic entrapment, the force fluid flow acting on clot material 99 is transmitted from the capture structure 115 to support frame 126 securing the capture structure 115. The force acting on the support frame 126 and in turn the support members 105, 110 urges the end 104 into the lumen wall. This action effectively fixes the second support frame 128. The force acting on the support frame 126 causes the angle β associated with the support frame 126 to increase the support frame 126 wedges further into the lumen wall.

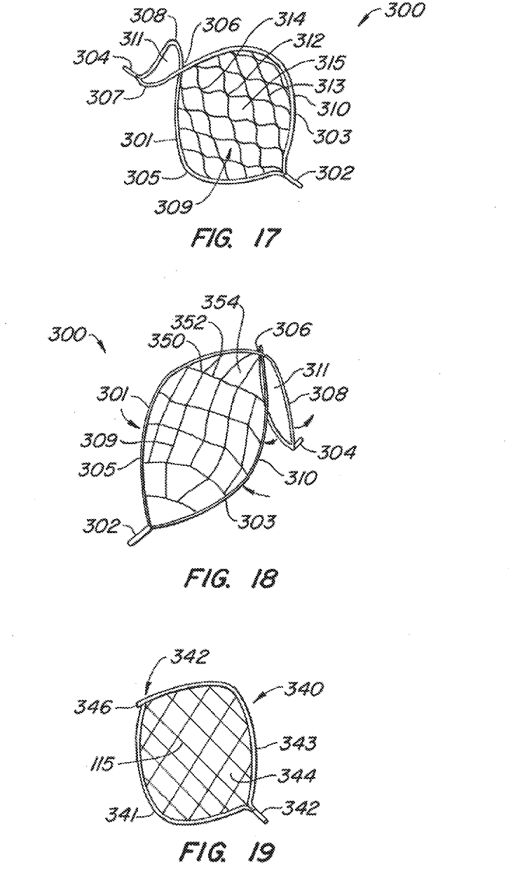

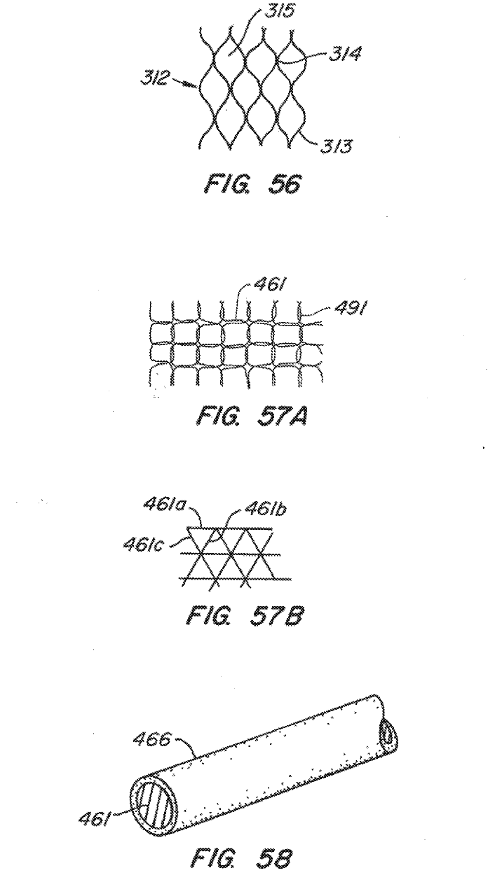

FIGS. 17, 18, and 19 illustrate various alternative filter device embodiments with support structures of different size and that may not be in contact with the lumen wall. FIG. 17 illustrates a perspective view of a filter device 300 according to one embodiment of the present invention. In this embodiment, elongate members 305, 310 are joined at ends 302, 304, to form frame 309 from end 302, sections 301, 303 and crossover 306 and frame 311 from end 304, sections 307, 308 and cross over 306. The frame 309 supports another embodiment of a material capture according to the present invention. The illustrated material capture structure 312 includes a plurality of strands 313 joined 314 to form a plurality of filter cells 315. The strands 313 may be joined using processes described below (e.g., FIG. 53A-53D) or may be formed by extruding the desired shape and size filter cell 315 from a material (e.g., FIG. 56).

FIG. 17 illustrates a so-called capacitor design because the elongate members that form frame 311 are configured to expand and contract the size and shape of frame 311 in response to changes in frame 309. This design feature allows an embodiment of the present invention to accommodate a large range of sizing and diameter changes. FIG. 18 illustrates an embodiment of the filter device 300 having a capture structure 350 having filter cells 354 formed by intersecting strands 352. FIG. 18 illustrates how inward movement of the frame 309 (indicated by the arrows) is corresponds to outward movement (indicated by the arrows) in the frame 308.

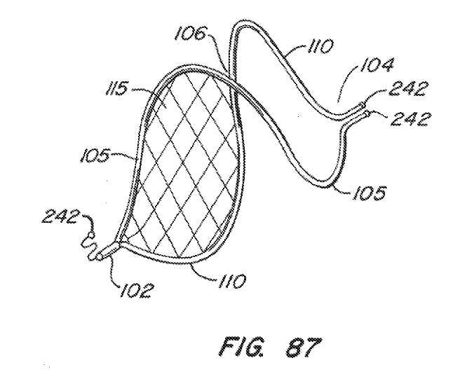

FIG. 19 illustrates an alternative filter device embodiment where the second frame is not closed. The filter device 340 includes support members 341, 343 that form a rounded support frame 344 to support the material capture device 115. The support members 341, 343 extend some distance beyond the cross over 342 but are not joined to form another end. A portion 346 of the support member 343 is shown extending beyond the cross over 342. The support members 341, 343 may extend for some distance along the device axis after the cross over 342 and may follow the same or a different shape as the shape of the support members in frame 309. The support members may extend along the device axis similar to earlier described two loop embodiments but stop short of being joined at a second end (e.g., FIG. 87).

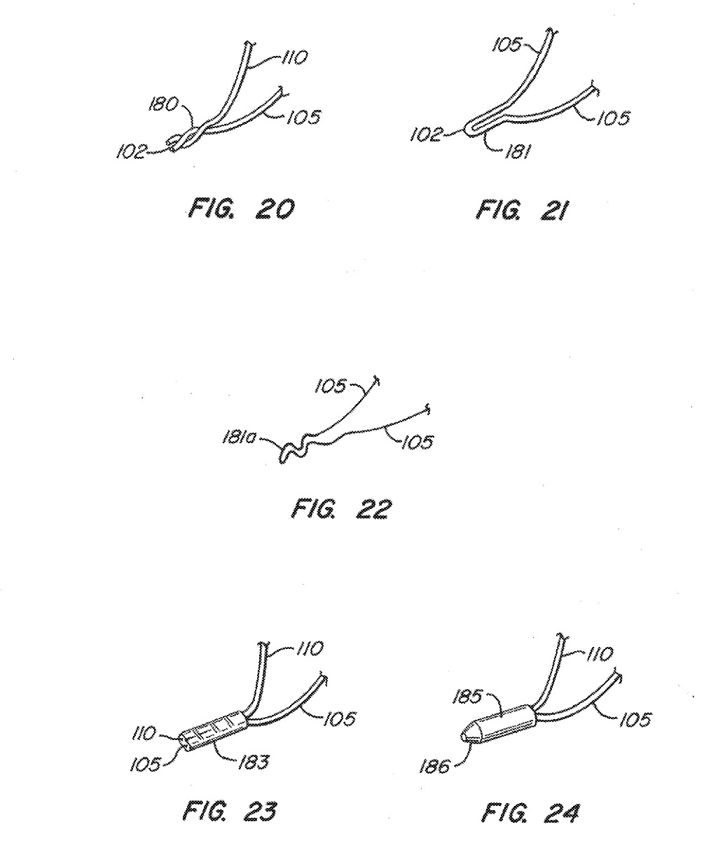

The ends of the filter devices of the present invention may be formed in a number of ways. A portion of the support structures 105, 110 may be wound 180 around one another (FIG. 20). In the illustrated embodiment, the wound portion 180 is used to form the end 102. In another alternative, the filtering device is formed from a single support member 105 that loops back on itself. In the illustrative embodiment of FIG. 21, support member 105 is formed into loop 181 to form the end 102. In an alternative to loop 181, the loop may contain a plurality of undulations (i.e., loop 181a in FIG. 22) or be formed into the shape of a retrieval feature or other component of the filter device. In yet another alternative, a cover is used to clamp, to join or otherwise bond the structural members together. In the illustrative example of FIG. 23, a generally cylindrical cover 183 is used to join together members 105, 110. The cover 183 may use any conventional joining method to secure the support members together such as adhesive, welding, crimping and the like. An alternative tapered cover 185 is illustrated in the embodiment of FIG. 24. The tapered cover 185 has a cylindrical shape and a tapered end 186. The tapered end 186 around the end having the tapered cover 185 and facilitates deployment and retrieval of the device. In one embodiment, the cover 185 is made of the same material as the structural member and/or the retrieval feature.

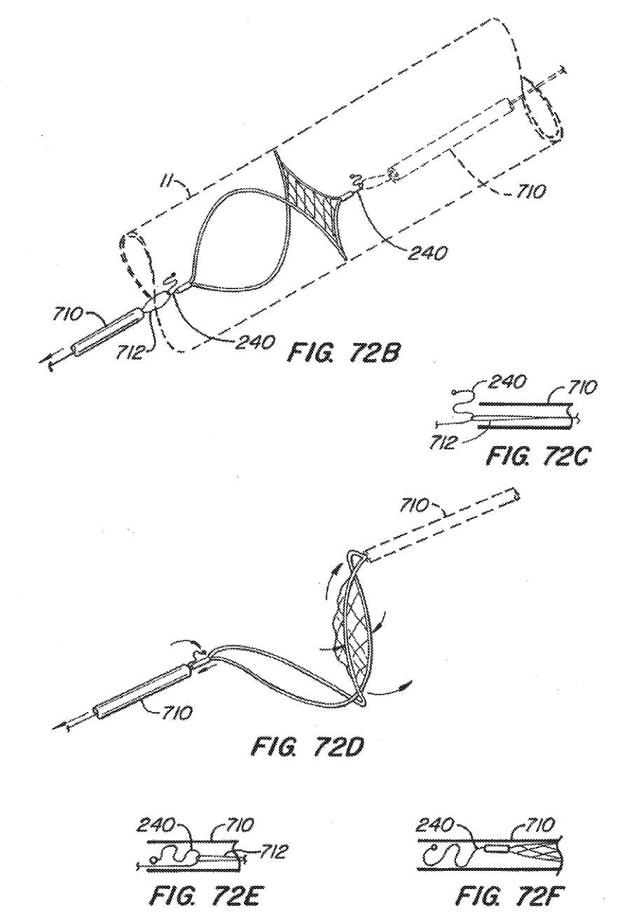

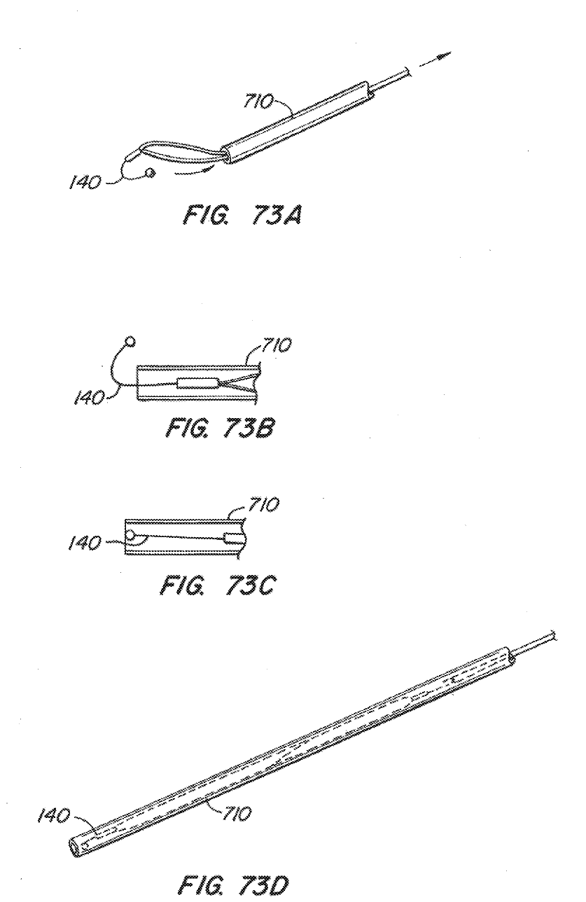

Some filter device embodiments of the present invention may include one or more retrieval features to assist recapturing and partially or fully recovering a deployed filter device. Retrieval features may be placed in any of a number of positions on the device depending upon the specific filter device design. In one embodiment, the retrieval device is positioned not only for ease of device recovery but also attached to the device in such a way that pulling on the retrieval device actually facilities removal of the device. In one embodiment, pulling on the retrieval device pulls the structural members away from the lumen wall. These and other aspects of the cooperative operation of the retrieval features during deployment and recapture will be described below with regard to FIGS. 72A-73D.

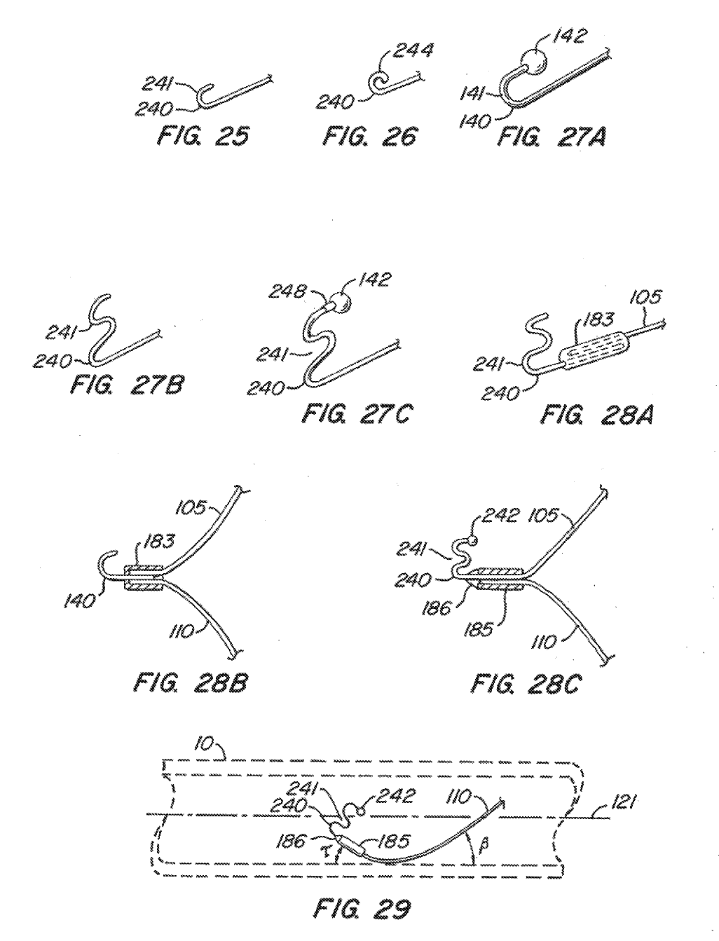

Several alternative embodiments of retrieval devices of the present invention are illustrated in FIGS. 25, 26, and 27A-27C. FIG. 25 illustrates a retrieval device 240 with a simple curve 241 formed in the end. FIG. 26 illustrates a retrieval device 240 with a curve 244 that is has a sharper radius of curvature than the curve 241 in FIG. 25. FIG. 27A illustrates a retrieval feature 140 having a curved section 141 with an atraumatic end 142. In the illustrative embodiment, the atraumatic end 142 is a ball than may be added to the end of curve 141 or formed on the end of the member used to form the feature 140. A ball 142 may be formed by exposing the end of the curved section 141 to a laser to melt the end into a ball. FIG. 27B illustrates a retrieval feature with a plurality of curved sections 241. In one embodiment, the curved sections 241 have a generally sinusoidal shape. In another embodiment, the curved sections 241 are configured to collapse when pulled on by a retrieval device like a snare (i.e., FIGS. 71A, 71B) FIG. 27C illustrates a retrieval feature 240 having a plurality of curved sections 241 and a ball 142 formed on the end. In additional embodiments, retrieval features of the present invention may include markers or other features to help increase the visibility or image quality of the filter device using medical imaging. In the illustrative embodiment of FIG. 27C, a radio opaque marker 248 is placed on the curved section 241. The marker 248 may be made from any suitable material such as platinum, tantalum or gold.

A cover placed about the ends may also be used to join a retrieval feature to an end or two support members. A cover 183 may be used to join a retrieval feature 240 to a support member 105 (FIG. 28A). In this illustrative embodiment, the support structure 105 and the retrieval feature 240 are separate pieces. A cover 183 may also be used to join together two members 110, 105 to a retrieval feature 140 (FIG. 28B). In another alternative embodiment, the retrieval feature is formed from a support member that is joined to the other support member. In the illustrative embodiment of FIG. 28C, the support member 105 extends through the tapered cover 185 and is used to form a retrieval feature 240. The tapered cover 185 is used to join the first support member and second support member 105, 110. In one alternative of the embodiment illustrated in FIG. 28C, the diameter of the support member 105 is greater than the diameter of the retrieval feature 240. In another embodiment, the diameter of the retrieval feature 240 is less than diameter of the support member 105 and is formed by processing the end of the support member down to a smaller diameter and is then shaped to form the retrieval feature 240. In another embodiment, the ball 242 or other atraumatic end is formed on the end of the retrieval feature.

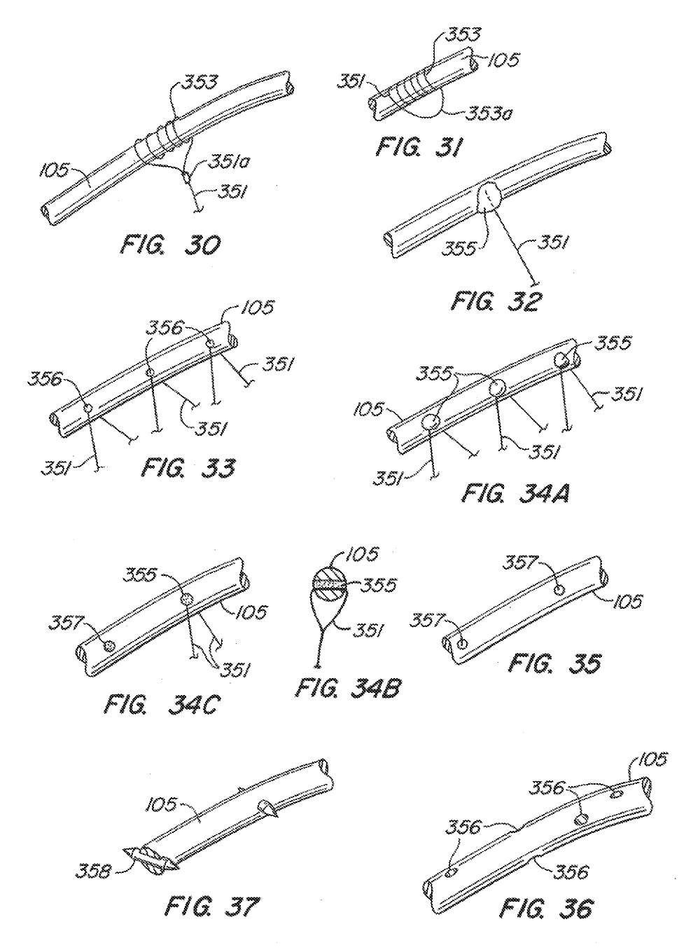

FIG. 29 illustrates a partial side view of a filter device in a lumen 10. This figure illustrates the retrieval feature angle τ formed by the retrieval feature and the interior lumen wall. The retrieval feature angle τ is useful in adjusting the height and orientation of the retrieval curves 214 and ball 242 within the lumen to improve the retrievably of the device. Generally, retrievably improves as the retrieval feature moves closer to the device axis 121 (i.e., central to the lumen axis as well). Additional curves may be added to the support members 110, 105 as needed to provide the desired range of retrieval feature angles. In one embodiment, τ ranges from −20 degrees to 90 degrees. In another embodiment, τ ranges from 0 degrees to 30 degrees.

### Attachment of Material Capture and Other Filtering Structures to Support Structures

A number of different techniques may be used to attach material capture structures to support members. For clarity, the material capture structure has been omitted from the illustrations that follow but would be suitably secured using the line 351 or a loop. In FIG. 30 illustrates a line 351 with a number of turns 353 about a support member 105. The line 351 is secured back onto itself using a clip 351a. FIG. 31 illustrates a line 351 with a number of turns 353 about the support member 105 to secure a loop 353a that may be used to tie off or otherwise secure a material capture structure. A line 351 may also be glued 355 to a support 105 (FIG. 32). In another alternative embodiment, holes 356 formed in the support member are used to secure one or more lines 351 that are used in turn to secure a material capture structure (FIG. 33). In an alternative to the linear arrangement of holes 356, FIG. 36 illustrates how holes 356 may be provided in a number of different orientations to assist in securing a material capture to the support structure 105. Alternatively, the line 351 may be glued 355 into the hole 356 (FIG. 34A and in section view 34B).

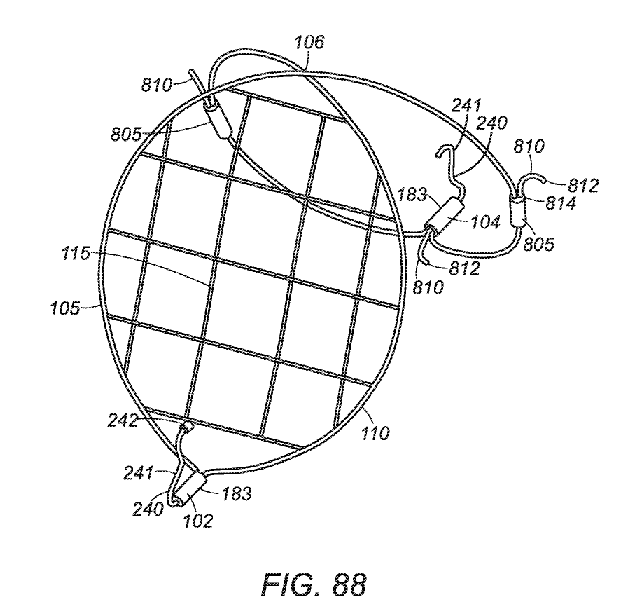

In other alternative embodiments, the holes 356 are used to secure lines 351 as well as provide a cavity for another material to be incorporated into the support structure 105. Other materials that may be incorporated into the support structure 105 include, for example, a pharmacological agent or a radio opaque material. The use of a radio opaque marker may be useful, for example, when the support structure is formed from a material with low imaging visibility such as, for example, shape memory polymers or biodegradable polymers. FIG. 34C illustrates an embodiment where one hole 356 is used to secure a line 351 and the other is filled with material or compound 357. In another alternative, some or all of the holes 356 may be filled with another material as in FIG. 35. In yet another alternative, the holes 356 are filled with small barbs 358 that may be used to secure the device to the lumen wall. The illustrative embodiment of FIG. 37 the barbs 358 are only long enough to break the surface of the lumen interior wall and not pierce through the lumen wall. While each of the above has been described with regard to the support member 105, it is to be appreciated that these same techniques could be applied to the support member 110 or other structure used to support a material capture structure. Additional alternative embodiments of hooks, barbs or other fixation devices or elements are described below with regard to FIGS. 88-126D.

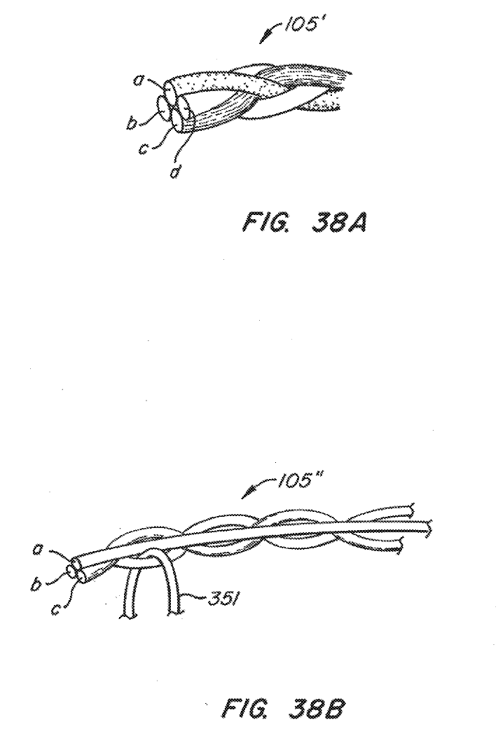

It is to be appreciated that the support structure embodiments are not limited to single member constructions. FIG. 38A illustrates an alternative braided support member 105′. Braided support structure 105′ is formed by 4 strands a, b, c, and d. FIG. 38B illustrates another alternative braided support member 105″. Braided support structure 105″ is formed by 3 strands a, b, and c. FIG. 38B also illustrates how the braid structure may be used to secure a line 351. As can be seen in this embodiment, by using the line 351 a material capture structure (not shown) is secured to at least one strand within the braided structure 105″.

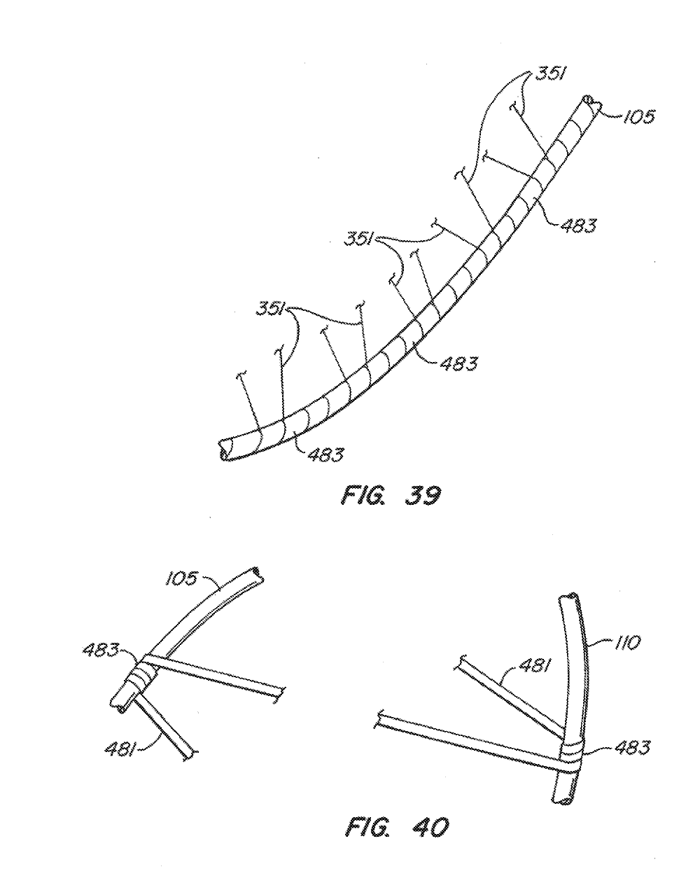

FIGS. 39 and 40 illustrate additional alternative techniques to secure a filter support structure to a support member. As illustrated in FIG. 39, there is illustrated a technique to secure a material capture structure securing line 351 to a support frame 105 using a material 481 wrapped around the support frame 105. In this manner, the material capture structure (not shown but attached to the lines 351) is attached to a material 481 that at least partially covers the first support structure 105. The lines 351 are passed between the material 481 and the support structure 105 as the material 481 as wraps 483 are formed along the support structure 105. The lines 351 are omitted in the embodiment illustrated in FIG. 40 as the material 481 forms wraps 483 and is used to secure the material capture structure (not shown). In one embodiment, the material 481 forms a tissue ingrowth minimizing coating over at least a portion of support structure. Alternatively, the filtering structure (not shown) is attached to the support structure 105 using a tissue ingrowth minimizing coating 481.

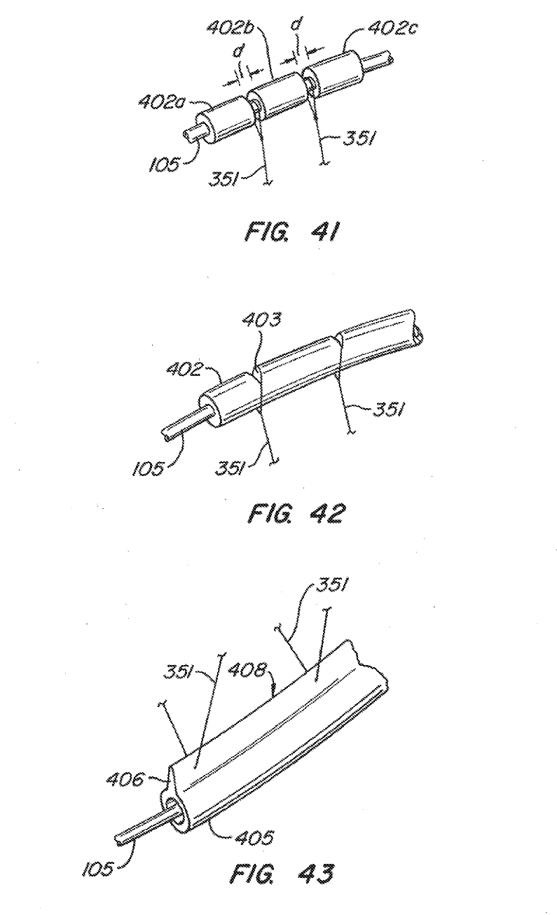

FIGS. 41, 42 and 43 relate to securing the material capture structure to a lumen disposed around the support member. FIG. 41 illustrates a lumen 402 that has been cut into segments 402a, 402b, 402c that are spaced by a distance “d.” Lines 351 are attached around the support member and in the space “d” between adjacent segments. The segments may remain apart or be pushed together to reduce or eliminated the spacing “d.” In contrast the segments in FIG. 41, the lumen 402 in FIG. 42 provides notches 403 for securing line 351. FIG. 43 illustrates a lumen 405 having a tissue growth inhibiting feature 408 extending away from the support member 105. As seen in section view 406 the inhibiting feature 408 has a different cross section shape than the support member 105. In addition, in some embodiments, the lumen 405 is selected from a suitable tissue ingrowth minimizing material so that is acts like a tissue ingrowth minimizing coating on the support structure. In other embodiments, the cross section shape 406 is configured to inhibit tissue growth over the tissue ingrowth minimizing coating.

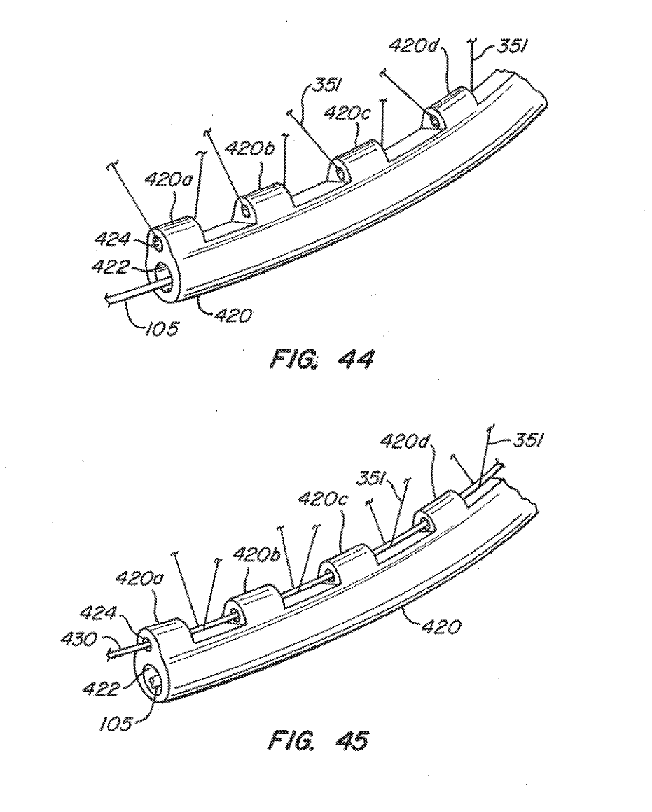

FIGS. 44 and 45 illustrate filter device embodiments utilizing dual lumen structures. The dual lumen structure 420 includes a lumen 422 and a lumen 424 and has a generally teardrop shaped cross section area. In this illustrative embodiment, the support structure 105 is disposed in the lumen 422 and the second lumen 424 is used to hold lines 351 and secure a material capture device (not shown). In the illustrative embodiment, the lumen structure 420 has been cut to form a number of segments 420a, b, c and d in the lumen 424. The connection rings formed by the segments 420a-d are used to secure lines 351 as needed. FIG. 45 illustrates an alternative configuration for the lumen structure 420. In this alternative configuration, a release line 430 extends through the notched lumen 424. The lines 351 extend about the release line 430 and hence to secure the material capture structure (not shown). Since the lines 351 are connected using the release line, removal of the release line from lumen 424 will allow the material capture structure secured using the lines 351 to be released from the support structure and removed from the lumen. A configuration such as that shown in FIG. 45 provides a filtering structure that would be releasably attached to an open loop (i.e., an open loop frame formed by the support structure). The embodiment illustrated in FIG. 45 provides a release line 430 positioned along the open loop (formed by member 105) and a filtering structure (not shown) is attached to the open loop using the release line.

In another embodiment, a filter device of the present invention is configured to be a coated endoluminal filter. In addition to coating all or a portion of the support structures or filter elements of this device, the coating on the support members may also be used to secure a filtering structure to the support structure. In one embodiment, a coated endoluminal filter has a support structure, a filtering structure attached to the support structure and a coating over at least a portion of support structure. In one aspect, the coated support structure may form a rounded support frame, an open loop or other structure to support a filtering structure described herein. In one embodiment, the coating over at least a portion of support structure is used to secure a plurality of loops (i.e., flexible form or rigid form) to the support structure. The plurality of loops are then used to secure a filtering structure such as a material capture structure, for example, within the coated endoluminal filter. In one embodiment, the coating is a tissue ingrowth minimizing coating.

It is to be appreciated that a filtering structure may also be attached to the support structure using the tissue ingrowth minimizing coating. In some embodiments, the tissue ingrowth minimizing coating is wrapped around the support structure or, alternatively, it may take the form of a tube. If a tube is used, the tube may be a continuous tube or comprise a plurality of tube segments. The tube segments may be in contact or spaced apart. The tube may have the same or different cross section shape than the support member. In another embodiment, the tissue ingrowth minimizing coating is in the shape of a tube and the support structure is in the interior of the tube.

In some other embodiments, a bonding material is provided between the tissue ingrowth minimizing coating and the support structure. The bonding material may be wrapped around the support structure or may take the form of a tube. If a tube is used, the tube may be a continuous tube or comprise a plurality of tube segments. The tube segments may be in contact or spaced apart. The bonding material tube may have the same or different cross section shape than the support member or the coating about the bonding material. In one embodiment, the bonding material is in the shape of a tube with the support member extending through the bonding material tube lumen. In one embodiment, a plurality of loops (i.e., flexible form or rigid form) are secured to the support structure by sandwiching the line used to form the loops between a bonding material around the support member and a coating around the bonding material. In one embodiment, the bonding material has a lower reflow temperature than the coating around the boding material. In this embodiment, the line used to form the loops is secured at least in part by reflowing the bonding material to secure the line between the coating around the bonding material and the support structure. In another alternative, the coating around the bonding material is a shrink fit coating that also shrinks around the bonding structure and the support member during or after a process that reflows the bonding material. In any of the above alternatives, the plurality of loops may be used to secure a filtering structure such as a material capture structure, for example, within the coated endoluminal filter.

Some embodiments of the coated endoluminal filter include some or all of the other features described herein such as, for example, a retrieval feature on the support structure, a retrieval feature on each end of the support structure, a support structure having two elongate bodies that are joined together to form a rounded frame, and a support structure having two spiral shaped elongate bodies. In addition, some coated endoluminal filters have a support structure that is generally symmetrical about a plane that is orthogonal to the flow direction of the filter and contains a crossover point. In another alternative coated endoluminal filter embodiment, the support structure of the coated endoluminal filter is generally symmetrical about a plane that is parallel to the flow direction of the filter and contains both ends of the support structure.

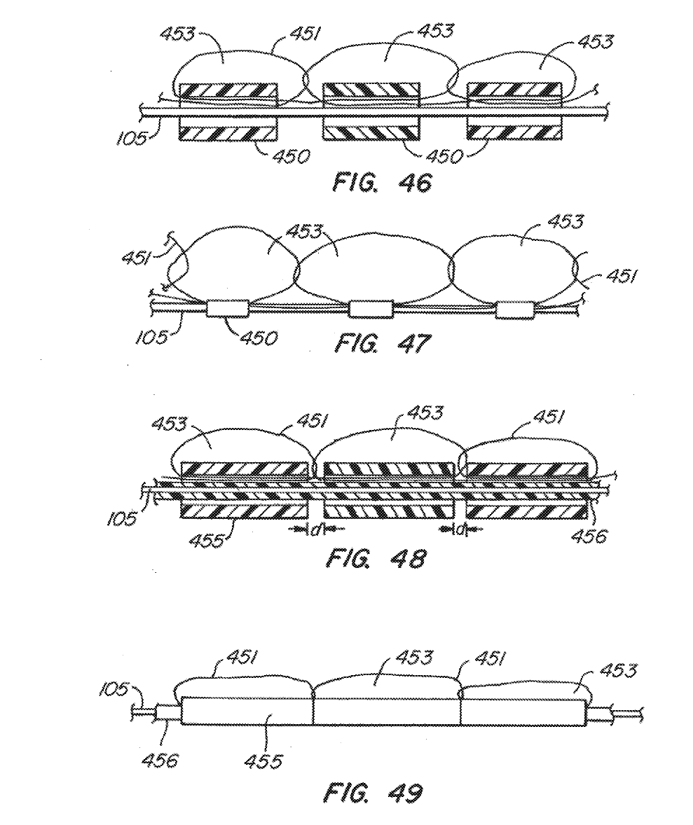

FIGS. 46-50, and 51A-51B illustrate several aspects of coated endoluminal filter embodiments. These figures are not to scale and have exaggerated dimensions to make clear certain details. FIG. 46 illustrates a number of segments 450 of a coating placed about the support member 105. One or more lines 451 extend between the segment 450 and the support member 105 and form a plurality of loops 453. In one embodiment, the line 451 is a single continuous line. Once formed, the segments 450 undergo suitable processing to shrink the segment diameter around the line 451 and the support member 105 thereby securing the line 451 and loops 453 against the support structure (FIG. 47). The segment 450 is secured about the support member 105 as illustrated in the end view of FIG. 51A. The segments 450 in the embodiment shown in FIG. 47 are spaced apart. In other embodiments, the segments 450 may be in contact or have spacing different from that illustrated in FIG. 47. The sizes of the various components illustrated in FIGS. 46, 47 and 51A are exaggerated to show detail. The dimensions of one specific embodiment are: the support member 105 is a NiTi wire having an outside diameter of between 0.011″ and 0.015″; the segments 450 are 0.2″ long cut from a PTFE heat-shrink tubing having and a pre-shrunk outside diameter of 0.018″ and a wall thickness of 0.002″; the line 451 is monofilament ePTFE of an outer diameter of 0.003″ and the loops 453 have a nominal diameter of between about 0.1″ to about 0.4″.

FIGS. 48, 49 and 51B illustrate a bonding material 456 about the support member 105 and a number of segments 455 about the bonding material 456. One or more lines 451 extend between the segments 455 and the bonding material 456 and form a plurality of loops 453. In one embodiment, the line 451 is a single continuous line. Once formed, bonding material 456 and/or the segments 450 undergo suitable processing to secure the line 451 between the bonding material 456 and the coating 455 thereby securing the line 451 and loops 453 against the support structure (FIG. 49). The coating segment 450 and the bonding material 456 is secured about the support member 105 as illustrated in the end view of FIG. 51B. The segments 455 in the embodiment shown in FIG. 48 are spaced apart by spacing “d.” In other embodiments, the segments 455 may be in contact after processing (FIG. 49) or have spacing different from that illustrated in FIG. 48. In a preferred embodiment, the spacing between the segments 455 is removed by a portion of the boding material 456 flowing between and securing adjacent segments 455. The sizes of the various components illustrated in FIGS. 48, 49 and 51B are exaggerated to show detail. The dimensions of one specific embodiment are: the support member 105 is a NiTi wire having an outside diameter of between 0.011″ and 0.016″; the segments 455 are 0.3″ long cut from a PTFE heat-shrink tubing having a pre-shrunk outside diameter of 0.022″ and a wall thickness of 0.002″; the bonding material is a tube of FEP heat shrink tubing having a pre-shrunk outside diameter of 0.018″ and a wall thickness of 0.001″; line 451 is 0.002″ outer diameter PET monofilament and the loops 453 have a nominal diameter of between about 0.1″ to about 0.4″. It is to be appreciated that the segments 450, 455 and bonding material 456 may be formed, for example, from: ePTFE, PTFE, PET, PVDF, PFA, FEP and other suitable polymers. Moreover, embodiments of strands, lines, fibers and filaments described herein may also be formed from ePTFE, PTFE, PET, PVDF, PFA, FEP and other suitable polymers.

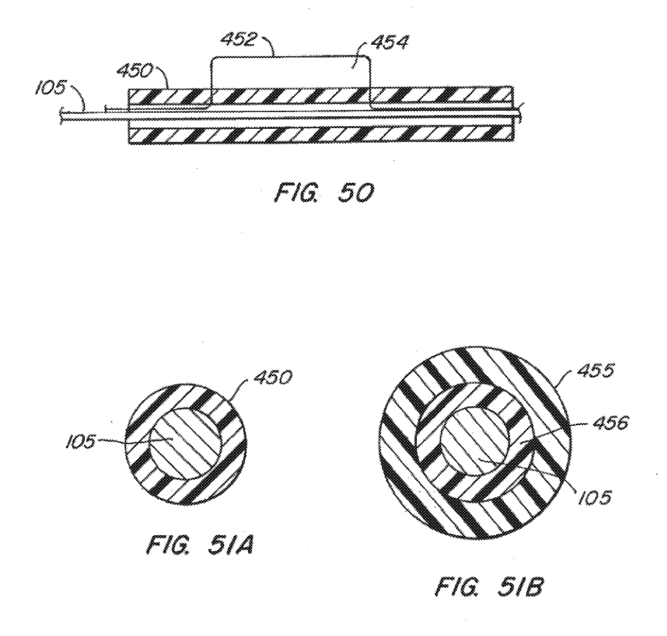

FIG. 50 illustrates the use of a continuous flexible line 452 passed through a continuous coating segment 450 forming loops 454. The loops 454 are disposed along the length of the coating 450 at regular intervals; the continuous coating segment 450 are uniform in length to the support members 105 using a PTFE heat shrink tubing having pre-shrunk diameter of 0.018″ and a wall thickness of 0.002″. The line 452 is monofilament ePTFE of an outer diameter of 0.003″ and the loops 454 have a nominal diameter of between about 0.1″ to about 0.4″.

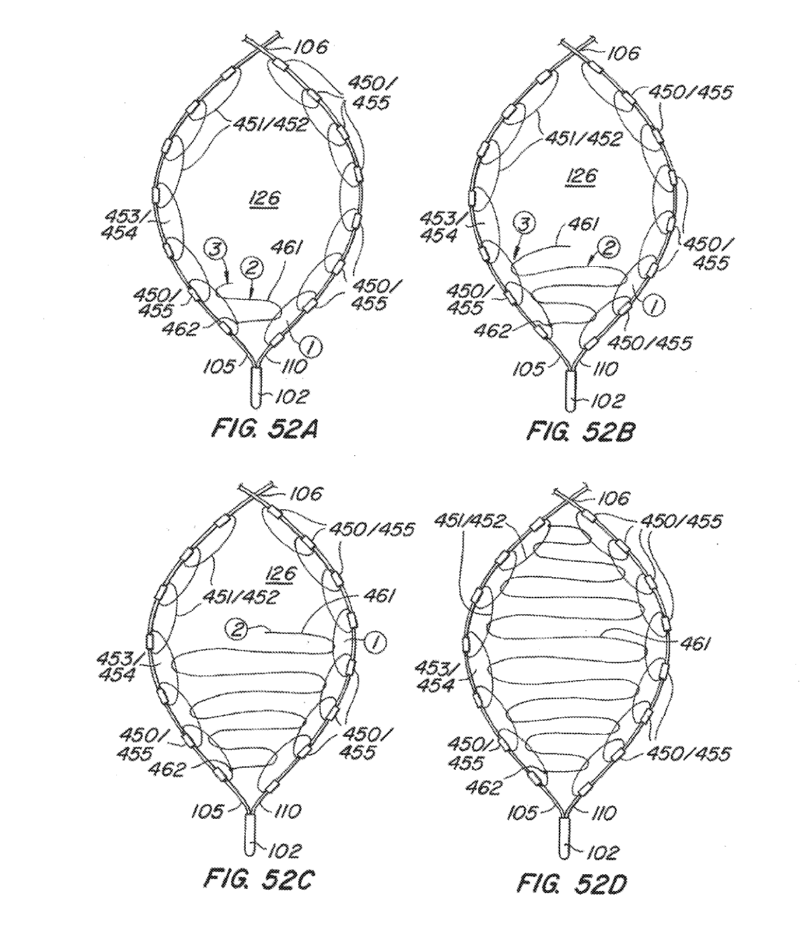

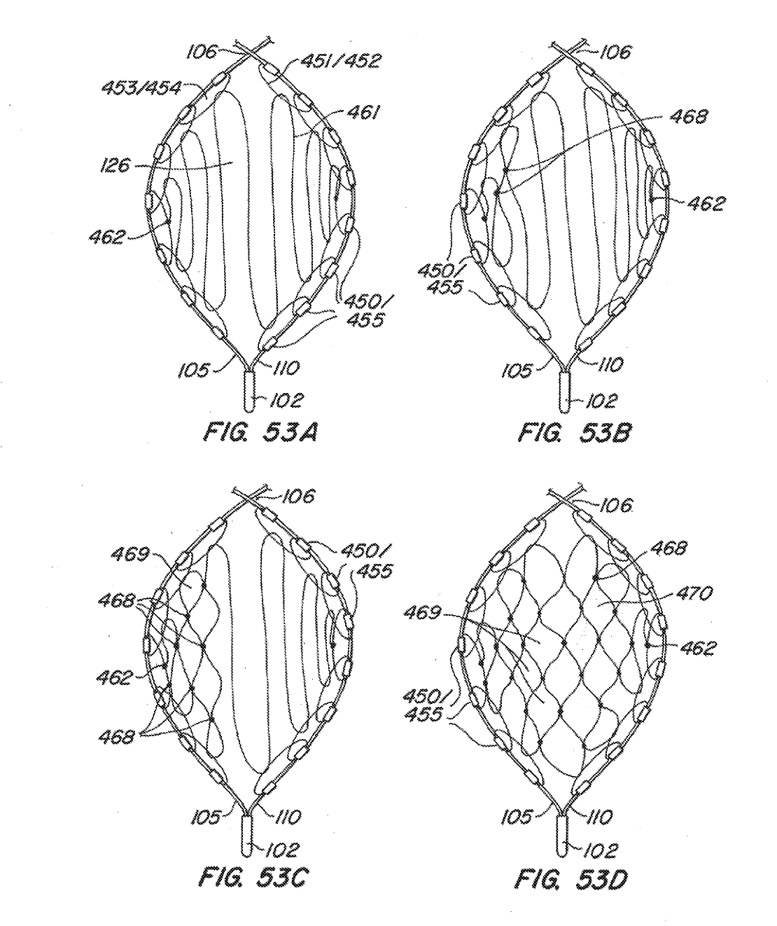

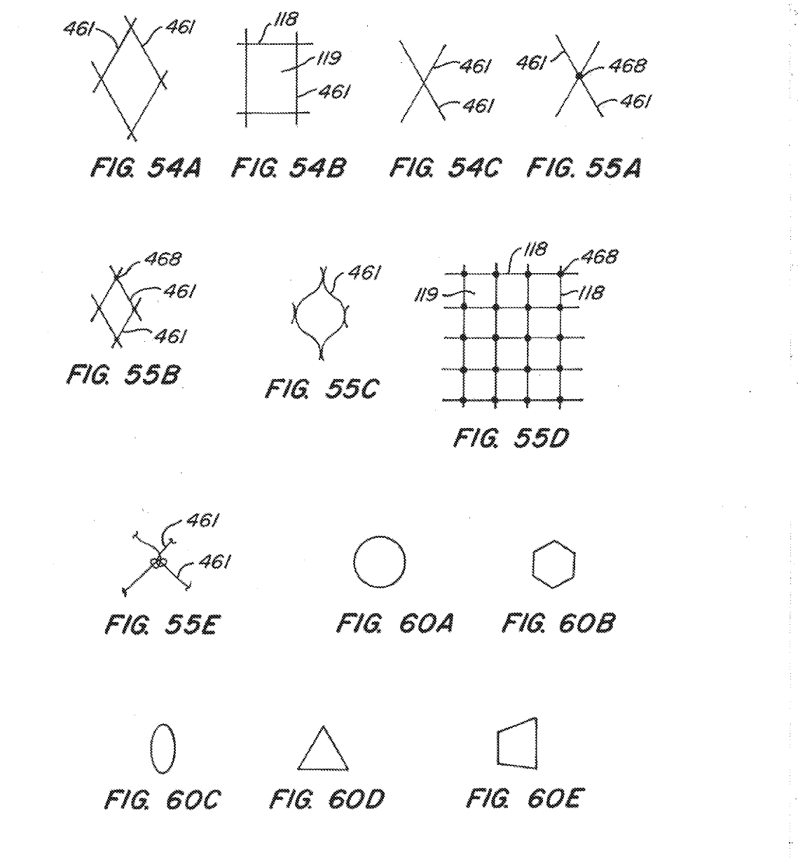

FIGS. 52A-52D, and 53A-53D illustrate alternative techniques for forming and/or attaching a filtering structure to a support structure. FIG. 52A illustrates an embodiment of a support frame 126 formed by support members 105, 110 between the end 102 and crossover 106 as described above. Loops 453/454 are formed using lines 451/452 as described above with regard to FIGS. 46-50, and 51A-51B. Thereafter, a filament 461 is suitably attached 462 to a line 451/452 by tying, welding, gluing or by incorporating the filament 461 during the processing steps described with regard to FIGS. 46-50, and 51A-51B. Next, the filament is traverses across the frame 126 and about the loops 453/454. In this embodiment, the lacing pattern between loops crosses a line extending between the end 102 and the crossover 106. The general pattern is that the filament extends across the frame 126 and around one right side loop (1) and back across the frame 126 (2) and around (3) a left side loop 453/454. The lacing process continues as shown in FIGS. 52B and 52C. When completed, the lacing process produces a filtering structure 465 from one or more filaments secured to loops 451/452 that are secured to the support members 105/110. The filament in the filtering structure 465 may be taut between the loops 451/452 or have some degree of sag (as illustrated in FIG. 52D). Filament 461 or other material used to form material capture structure may be coated with a pharmacological agent (coating 466 in FIG. 58). The pharmacological agent may be any of a wide variety of compounds, drugs and the like useful in the procedures performed using or the operation of various filtering device embodiments of the present invention. The pharmacological agent coating 466 may include pharmacological agents useful in preventing or reducing thrombus formation on the filtering structure, chemically lysing debris captured in the filtering structure and the like.

FIG. 53A illustrates an embodiment of a support frame 126 formed by support members 105, 110 between the end 102 and crossover 106 as described above. Loops 453/454 are formed using lines 451/452 as described above with regard to FIGS. 46-51B. Thereafter, a filament 461 is suitably joined 462 to a line 451/452 by tying, welding, gluing or by incorporating the filament 461 during the processing steps described with regard to FIGS. 46-50, 51A-51B. Next, the filament 461 was laced as described above with regard to FIG. 52A about the loops 453/454. In this embodiment, however, the lacing pattern between loops remains generally parallel to a line extending between the end 102 and the crossover 106. When completed, the lacing process produces a filtering structure from one or more filaments 461 that extend parallel to a line between the end 102 and crossover 106 and are secured to loops 451/452 secured to the support members 105/110. This filtering structure (FIG. 53A) may be used within a filter device of the present invention. In addition, the filtering structure in FIG. 53A (as well as the structure in FIG. 52D) may be further processed to join 468 adjacent filaments 461 to form filter cells 469 as part of a filtering structure 470. The process used to join 468 adjacent filaments 461 may include any conventional joining technique such as tying, welding, bonding, gluing, and the like. In addition, segments of tubing (i.e., segments 450, 455 456 described above) could be used to join 468 portions of adjacent filaments 461. In one specific embodiment, the filament 461 is ePTFE monofilament with an outer diameter of 0.003″ joined 468 using a piece of FEP heat shrink tubing having a pre-shrunk outer diameter of 0.008″ and a wall thickness of 0.001″. The filtering structure 470 may be taut between the loops 451/452 or have some degree of sag (as illustrated in by the filtering structure in FIG. 52D). The filter cells 469 may be formed in numerous sizes and shapes as described in greater detail below.

Alternatively, the filtering structures in FIG. 53A and FIG. 52D may incorporate additional loops 491 formed by looping the filament 461 as illustrated in FIG. 57A.

Alternative Filtering and/or Material Capture Structures

In some embodiments, the material capture structure contains a number of filter cells. Filter cells may be formed in a number of different ways and have a number of different shapes and sizes. The shape, size and number of filter cells in a specific filter may be selected based on the use of a particular filter. For example, a filter device of the present invention configured for distal protection may have a filter cell size on the order of tens to hundreds of microns to less than 5 millimeters formed by a selecting a filter material with a pore size (FIG. 63A, 63B) suited to the desired filtration level. In other applications, the filter cell may be formed by overlapping (i.e., joined or crossed without joining) filaments to form cells that will filter out debris in a lumen above a size of 2 mm. Various other filter sizes and filtration capacities are possible as described herein.

Intersecting filaments (FIG. 54C) may be used to form diamond shaped filter cells (FIG. 54A), as well as rectangular shaped filter cells (FIGS. 54B, 2A and 9B). Multiple strand patterns may also be used such as the three strand 461a, 461b and 461c array illustrated in FIG. 57B. Intersecting filaments may also be knotted, tied or otherwise joined 468 (FIGS. 55A and 55E). Intersecting filaments may form the same or different filter cell shapes such as, for example, an elongated oval in FIG. 55C, one or more joined diamonds as in FIG. 55B and an array of joined polygons as in FIG. 55D. Cells may also be formed using the techniques described above in FIGS. 52A-52D and 53A-53D. In one embodiment, a filter cell is defined by at least three intersecting filaments 461. The filter element 461 may be formed from any of a wide variety of acceptable materials that are biocompatible and will filter debris. For example, filaments, lines and strands described herein may be in the form of a multifilament suture, a monofilament suture a ribbon, a polymer strand, a metallic strand or a composite strand. Additionally, filaments, lines and strands described herein may be formed from expanded polytetrafluoroethylene (ePTFE), polytetrafluoroethylene (PTFE), Poly(ethylene terephthalate) (PET), Polyvinylidene fluoride (PVDF), tetrafluoroethylene-co-hexafluoropropylene (FEP), or poly(fluoroalkoxy) (PFA), other suitable medical grade polymers, other biocompatible polymers and the like.

The joined polygons may have any of the shapes illustrated in FIGS. 60A-60F. It is to be appreciated that filter cells may have any, one or more, or hybrid combinations of shapes such as, for example, circular (FIG. 60A), polygonal (FIG. 60B), oval (FIG. 60C), triangular (FIG. 60D), trapezoidal or truncated conical (FIG. 60E).

In addition, the material capture structure may have filter cells formed by extruding a material into a material capture structure. FIG. 56 illustrates an exemplary filtering structure 312 where a material is extruded into strands 313 that are joined 314 and spaced apart for form one of more filter cells 315. In one embodiment, the strands are extruded from Polypropylene material, forming diamond shaped filter cells approximately 4 mm in height and 3 mm in width.

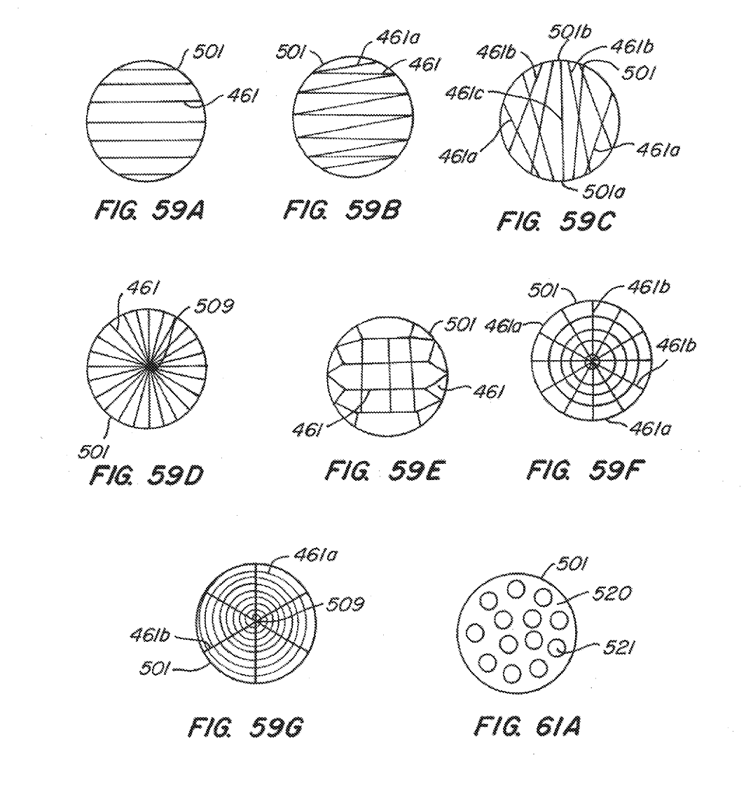

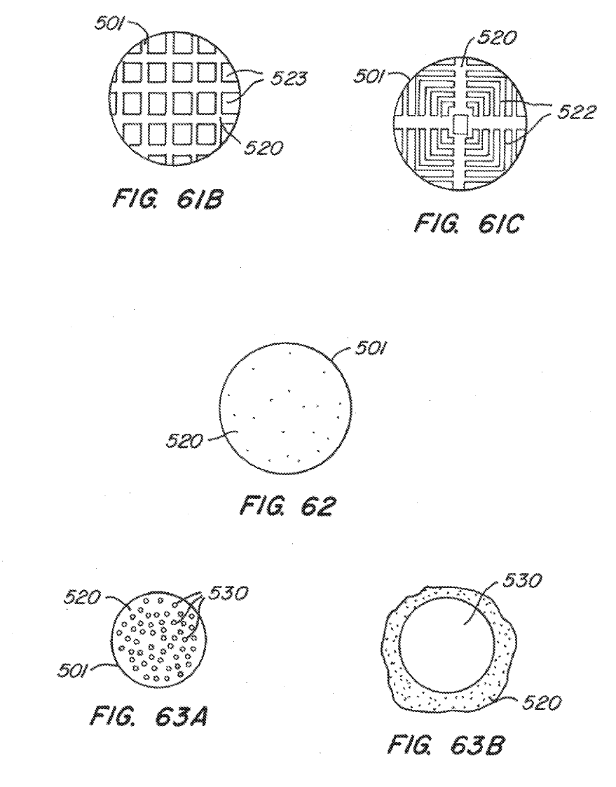

FIGS. 59A-59G, 60A-60E, 61A-61C, 62, and 63A-63B illustrate several different filtering structure configurations. For simplicity of illustration, the filtering material is shown attached to a circular frame 501. It is to be appreciated that the circular frame 501 represents any of the various open loop, rounded frame or other support frames described herein. FIG. 59A illustrates a frame pattern similar to FIG. 52D. FIG. 59B adds an additional transverse filaments 461a at an angle to the filaments 461. FIG. 59C illustrates a plurality of filaments 461a extending up from the frame bottom 501a about a central filament 461c and a plurality of filaments 461b extending down from the frame top 501b about a central filament 461c. In this illustrative embodiment, the filaments 461a,b are arranged symmetrically about the central filament 461c. Other non-symmetrical configurations are possible. More than one central filament 461c may be used to form a variety of different size and shaped polygonal filter cells (e.g., FIG. 59E).

Filaments may also be arranged using a variety of radial patterns. For example, multiple filaments 461 may from a common point 509 out the edge of frame 501. In some embodiments, the common point is central to the frame 501 (FIG. 59D) and in other embodiments the common point 509 is in a different, non-central location. The sectors formed by the multiple filaments (FIG. 59D) may be further divided into multiple filter cell segments by winding a filament 461a about and across segment filaments 461b. In contrast to a single filament spirally out from the point 509 as in FIG. 59G, the segmented filter cells in FIG. 59F are formed by attaching single filament 461a to the segment filaments 461b.

FIGS. 61A-C and FIG. 62 illustrate the use of a sheet of material 520 to form a filter structure. The material 520 may have any of a variety of shapes formed in it using any suitable process such as punching, piercing, laser cutting and the like. FIG. 61A illustrates a circular pattern 521 formed in material 520. FIG. 61B illustrates a rectangular pattern 523 formed in material 520. FIG. 61C illustrates a complex pattern 522 cut into material 522. It is to be appreciated that the material 520 may also be placed in the frame 501 without any pattern (FIG. 62). The illustrative embodiment of FIG. 62 may be useful for occluding the flow within a lumen. Suitable materials 520 for an occlusion application include for example, wool, silk polymer sheets, other material suited to prevent blood flow in a lumen when extended across a lumen and the like. Additionally, the filter material 520 may be a porous material having pores 530 (FIG. 63A). The material 520 may be selected based on the average size of individual pores 530 (FIG. 63B) depending upon the procedure or use of the filter device. For example, the material 520 may be any of the porous materials using in existing distal protection and embolic protection devices. In general, a wide variety of pore 530 sizes are available and may range from 0.010″ to 0.3″. Other pore sizes are also available depending upon the material 520 selected.

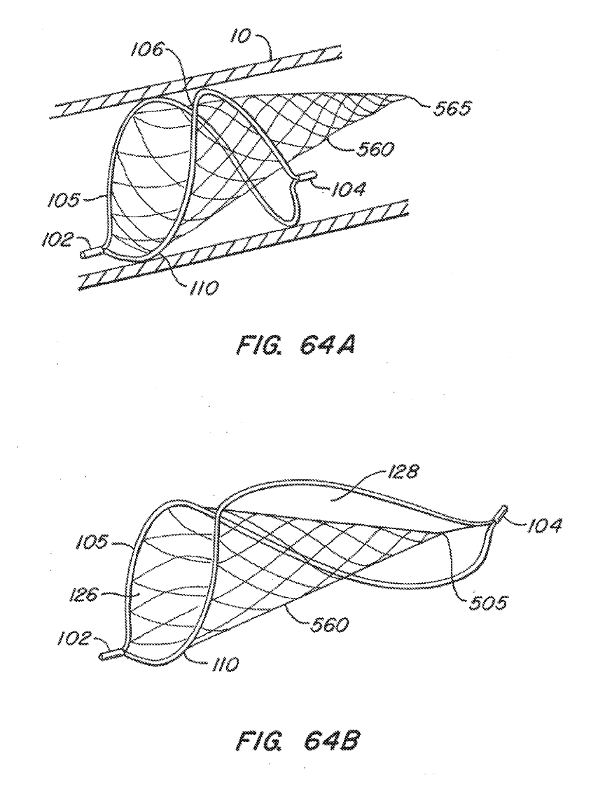

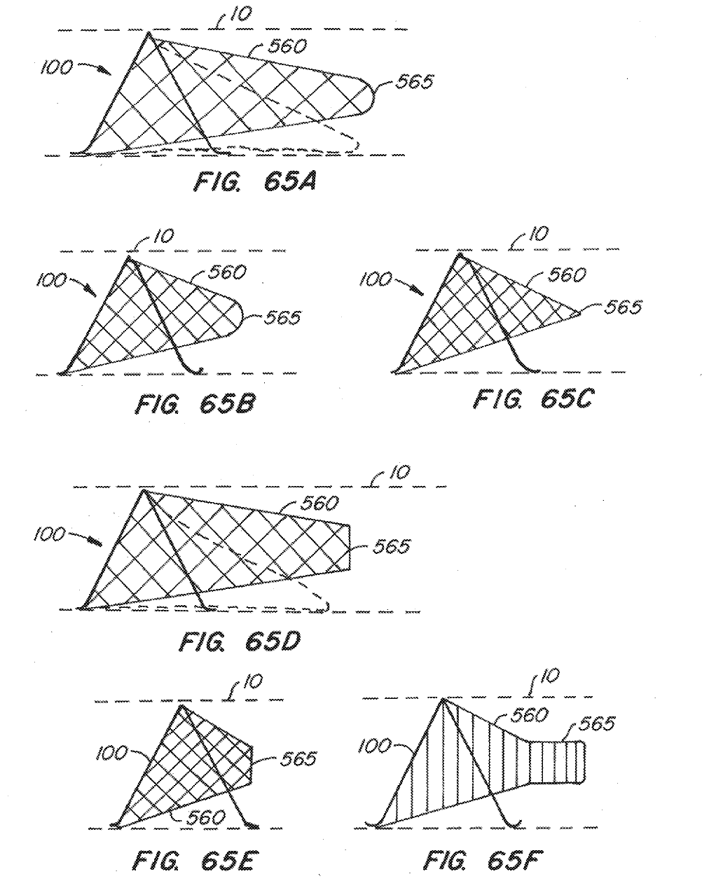

FIGS. 64A-64B and 65A-65F illustrate the use of nets or other web structures within the filtering device. The various net structure embodiments described herein are used as material capture structures within filter device embodiments of the present invention. Each of these alternative is illustrated in a support structure similar to that of device 100 in FIG. 2A and elsewhere. When deployed within the lumen 10, the material capture structure 560 has a defined shape such as a cone with a discrete apex 565 (FIG. 64A). In this embodiment, the net structure is long enough to contact the sidewall of the lumen 10 when deployed in the lumen 10. Alternatively, the apex 565 may be attached to the end 104 to keep the net 560 in the lumen flow path and out of contact with the lumen sidewall (FIG. 64B). The net 565 may also have a rounded apex 565 (FIG. 65A) or a truncated cone (flat bottom) (FIG. 65D). Alternatively, the net 560 may a discrete apex 565 so short that it will not contact the lumen sidewall when deployed (FIG. 65B). The short net may also have a rounded apex 565 (FIG. 65B), a flat apex (FIG. 65E) or a sharp apex (FIG. 65C). In addition, the net 560 may have a compound apex 565 (FIG. 65F).

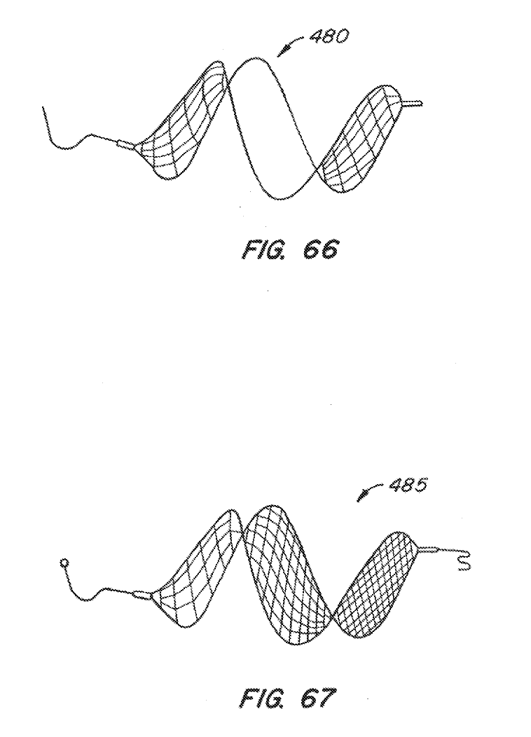

FIGS. 66 and 67 illustrate how various different features described above can be combined. For example, FIG. 66 illustrates a multi-support frame device 480 having a retrieval feature on only one end and an open frame (i.e., no filter structure). FIG. 67 illustrates an alternative multi-support frame device 485 having different retrieval features on each end, filter structures in each of the support structures and each of the filter structures having a different filter capacity. It is to be appreciated that the above described details of the construction, components, sizes, and other details of the various filter device embodiments described herein may be combined in a number of different ways to produce a wide array of alternative filter device embodiments.

### Delivery, Recovery and Repositioning of a Filtering Device

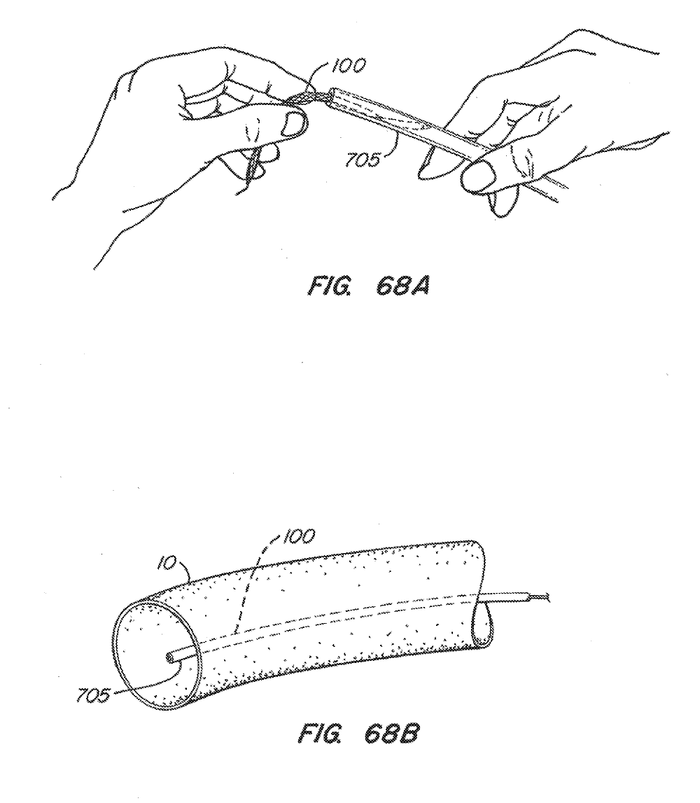

FIG. 68A illustrates an embodiment of the filter device 100 of the present invention loaded into an intravascular delivery sheath 705. The device 100 is illustrated and described above, for example, in relation to FIG. 16A. Using conventional endoluminal and minimally invasive surgical techniques, the device can be loaded into the proximal end of the sheath 705, before or after advancing the sheath 705 into the vasculature, and then advanced through the sheath using a conventional push rod. The push rod is used to advance the device 100 through the delivery sheath lumen as well as fix the position of the device (relative to the sheath 705) for device deployment. In one preferred technique, the device is loaded into the proximal end of a delivery sheath that has already been advanced into a desired position within the vasculature (FIG. 68B). The device 100 may be pre-loaded into a short segment of polymeric tubing or other suitable cartridge that allows the device 100 to be more readily advanced through a hemostasis valve.

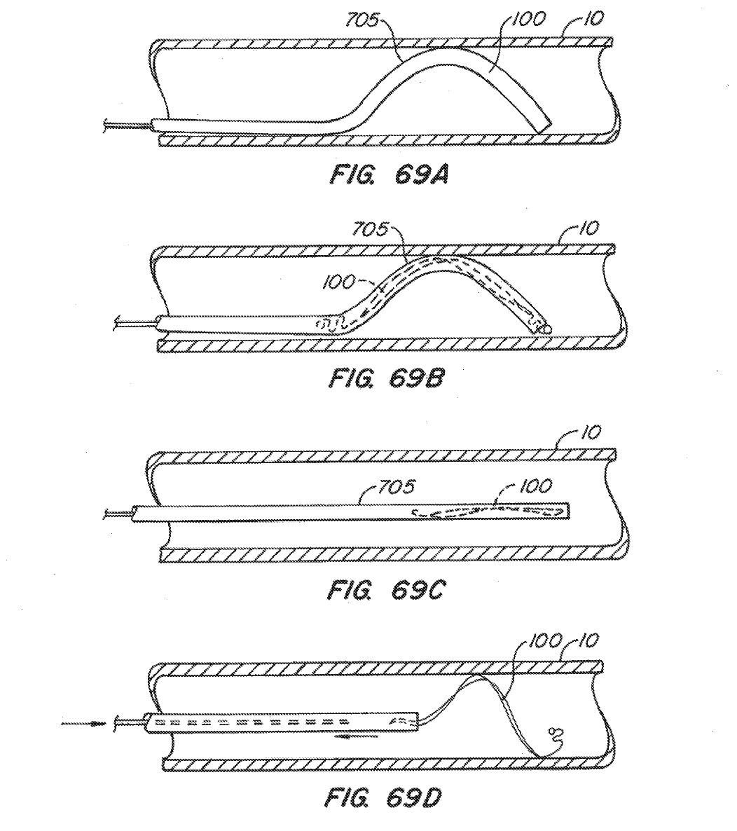

When used with a compliant delivery sheath 705, the pre-formed shape of the device 100 deforms the sheath to conform to the device shape (FIG. 69A, 69B). Accordingly, a flexible, compliant sheath 705 assumes the curvature of the stowed device. The deformation of the delivery sheath 705 helps stabilize the position of the sheath 705 in the vasculature and facilitates accurate deployment of the device 100 to the intended delivery site. In contrast, a non-compliant delivery sheath 705 (i.e., a sheath that is not deformed to conform to the preformed shape of the device 100) maintains a generally cylindrical appearance even through the device 100 is stowed within it (FIG. 69C). Regardless of the type of sheath used, device delivery is accomplished by using the push rod on the proximal side of the device to fix the position of the device within the sheath 705 and then withdrawing the sheath 705 proximally. As the device 100 exits the distal end of sheath 705, it assumes the pre-formed device shape (FIG. 69D).

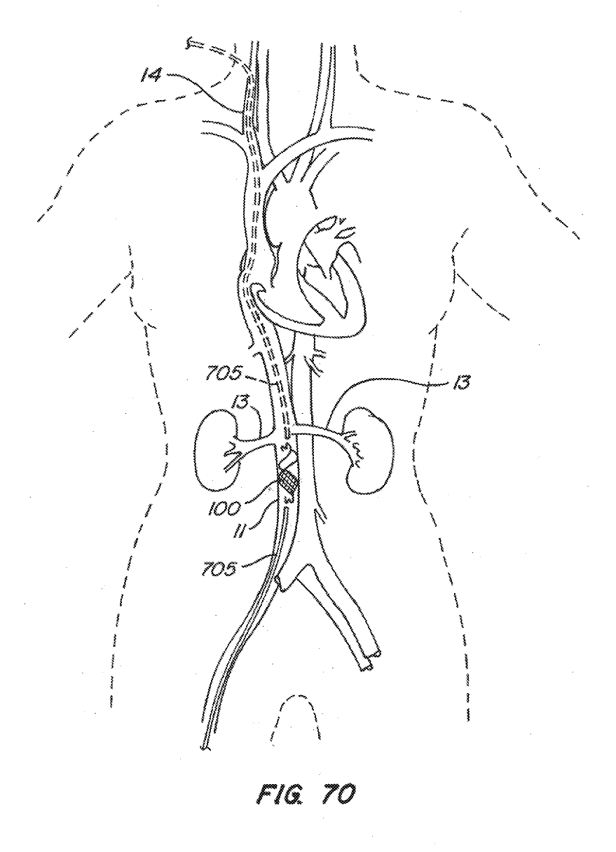

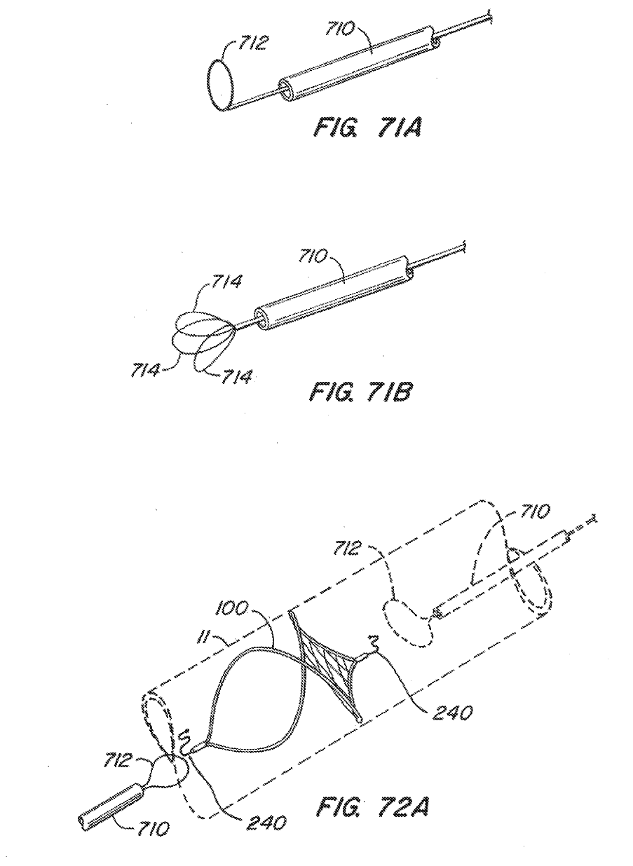

The symmetrical device shape (see e.g., devices in FIGS. 15 and 16A), facilitates the deployment and retrieval of the device from multiple access points in the vasculature. A device 100 is shown positioned in the vasculature within the inferior vena cava 11 immediately below the renal veins 13 (FIG. 70). Parts of the device, for example the support frame and/or the material capture structure, may be visualized during device deployment, retrieval, and repositioning to ensure proper positioning. The device may be visualized using conventional procedures such as fluoroscopy, ultrasound, and IVUS. A femoral access path (solid) and a jugular 14 access path (phantom) are illustrated. The femoral access path (solid) and a jugular access path may each be used for device deployment, repositioning and retrieval. Alternatively, the vena cava could be accessed via brachial or antecubital access for device deployment, repositioning and retrieval.

Retrieval of the devices is most preferably accomplished by endoluminal capture using one of the retrieval features described herein. (i.e., FIGS. 27A-E) The retrieval features described herein have been designed to work well using a commercially available snares two of which are illustrated in FIG. 71A and FIG. 71B. The single loop Gooseneck snare 712 is illustrated in FIG. 71 inside of a recovery sheath 710. The multiple loop Ensnare 714 is illustrated in FIG. 71B inside of a recovery sheath 710. These conventional snares are controlled by a physician using a flexible, integral wire.

The sequence of device recapture and removal from a body lumen (here the vena cava11) is illustrated in FIGS. 72A-C. In these figures, the solid lines are for a femoral recovery and the phantom lines are for a jugular recovery (e.g., FIG. 70). A collapsed snare is advanced via a delivery sheath to the proximity of the retrieval feature 240 (FIG. 72A). Once in place, the snare 712 is exposed and assumes a pre-defined expanded loop shape which is looped over the retrieval feature 240 as illustrated from either end in FIG. 72B. At least parts of the device may be visualized to aid in proper positioning of the snare.

The snared device 100 can then be either pulled into the sheath 710, or alternatively and more preferably, the recovery sheath 710 is advanced over the device 100 while maintaining positive control of the snare 712 as the sheath 710 advances over the device 100. Advancing the recovery sheath 710 over the device 100 facilitates atraumatic removal of the device 100 from any tissue that has grown in or around the device 100. The retrieval action, which tends to collapse the device radially inward (FIG. 72D), also facilitates removal from any tissue layer formed on the device. Recovering the filtering device by pulling on a flexible retrieval feature attached to the filtering device. Moreover, pulling on a portion of the filter structure (i.e., a retrieval feature) removes the opposing spiral elements from the lumen wall.

As the device is drawn into the sheath 710, the pre-formed shape of the device also urges the support members away from the lumen wall which also assists in atraumatic device removal.

The flexible retrieval element 240 assumes a collapsed configuration as it is being drawn into the recovery sheath as illustrated in FIG. 72C and FIG. 72E. Note that the retrieval feature 240 on the opposite end of the device assumes a straightened configuration as is drawn into the recovery sheath (FIG. 72F). An additional embodiment, in which a single curved retrieval feature 140 (FIG. 27A) is withdrawn into the delivery sheath 710 as shown in FIG. 73A. The distal retrieval feature (relative to the snare) assumes a straightened configuration FIG. 73C from a curved configuration FIG. 73B as is completely withdrawn into the sheath FIG. 73D.

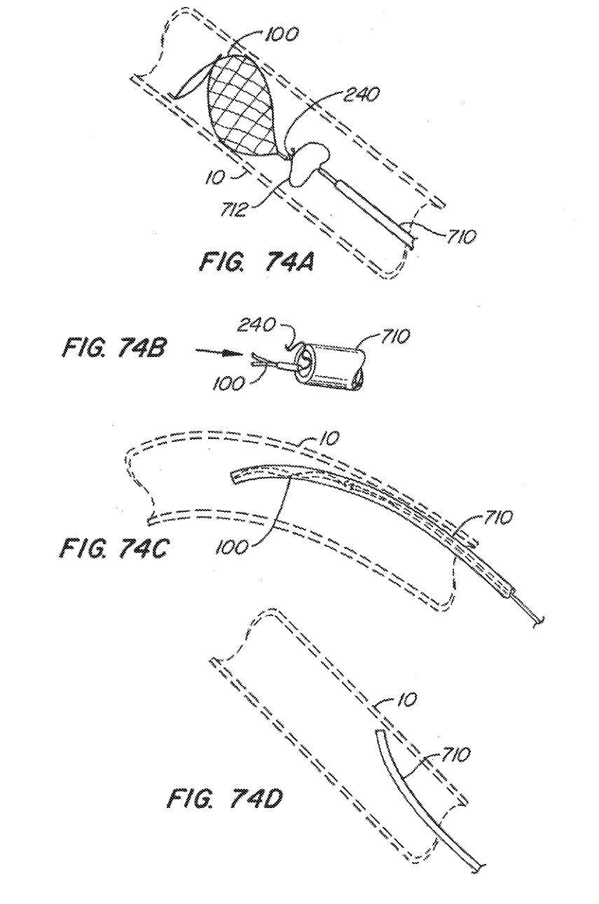

Additionally, repositioning the filter 100 from one lumen position to another is illustrated in FIGS. 74A-74D. Because of the atraumatic design of filter devices of the present invention, repositioning of the filter device 100 may be accomplished by fully recapturing (FIG. 74C) or only partially recapturing (FIG. 74B) the device 100 into a recovery sheath 710. The atraumatic design of the device 100 allows the device to simply secured by one end (FIG. 74B) and pulled along the lumen wall into the desired position and then released. The device may be visualized using, for example IVUS or fluoroscopy to aid in repositioning of the device. The delivery sheath and recovery sheath are provided with the same reference numbers since filter devices of the present invention may be deployed into and recovered from the vasculature using sheaths that are about the same size. As such, devices of the present invention may be deployed into the vasculature from a delivery sheath having a first diameter. Then, the device may be retrieved from the vasculature using a recovery sheath having a second diameter no more than 2 Fr larger than the first diameter (1 Fr=0.013″=⅓ mm). Alternatively, the second diameter may be no more than 1 Fr larger than the first diameter or, alternatively, the first diameter is about the same as the second diameter.

In a full recovery, the device is pulled completely into a recovery sheath (FIG. 74A), the sheath is repositioned from the original position (FIGS. 74A, 74C) to a second position (FIG. 74D) and deployed into the vasculature again (FIG. 69D). In the case where the snare wire columnar strength is insufficient to redeploy the device, the snare can be delivered within a secondary inner sheath within the retrieval sheath. This allows the positive control of the retrieval feature to be obtained, such as illustrated in FIG. 74B, the device withdrawn into the retrieval sheath and then redeployed with the inner sheath acting as a push rod.

### Various Methods of Using Filtering Devices

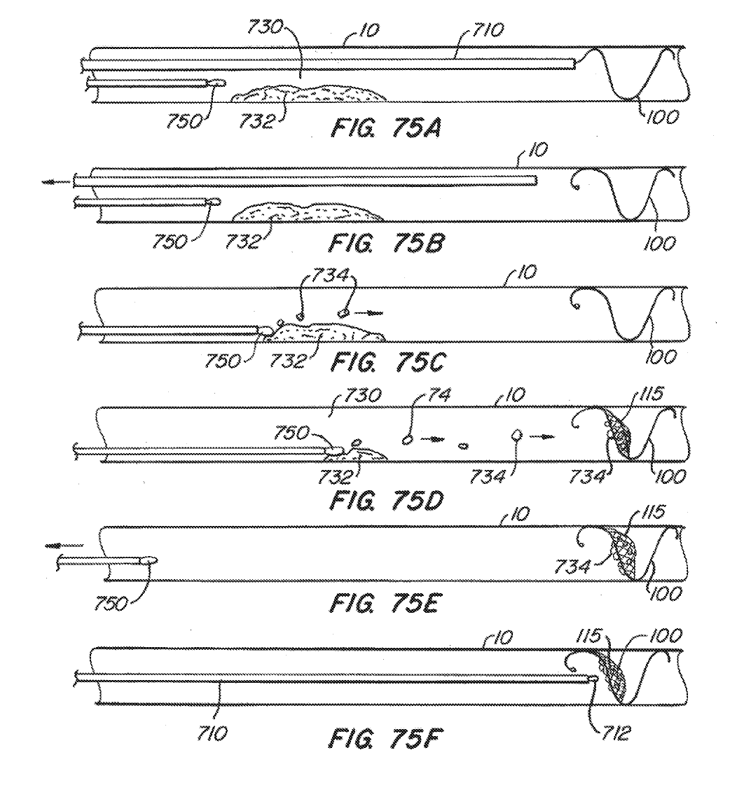

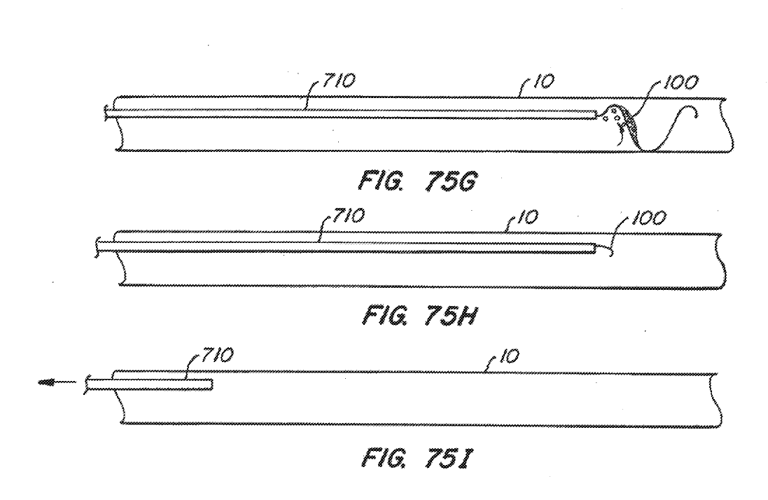

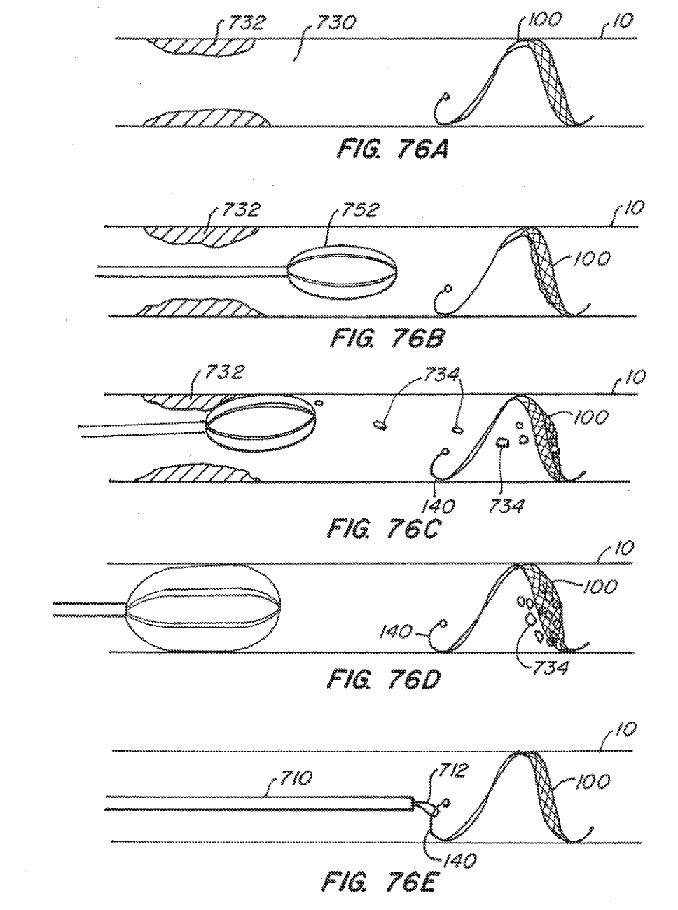

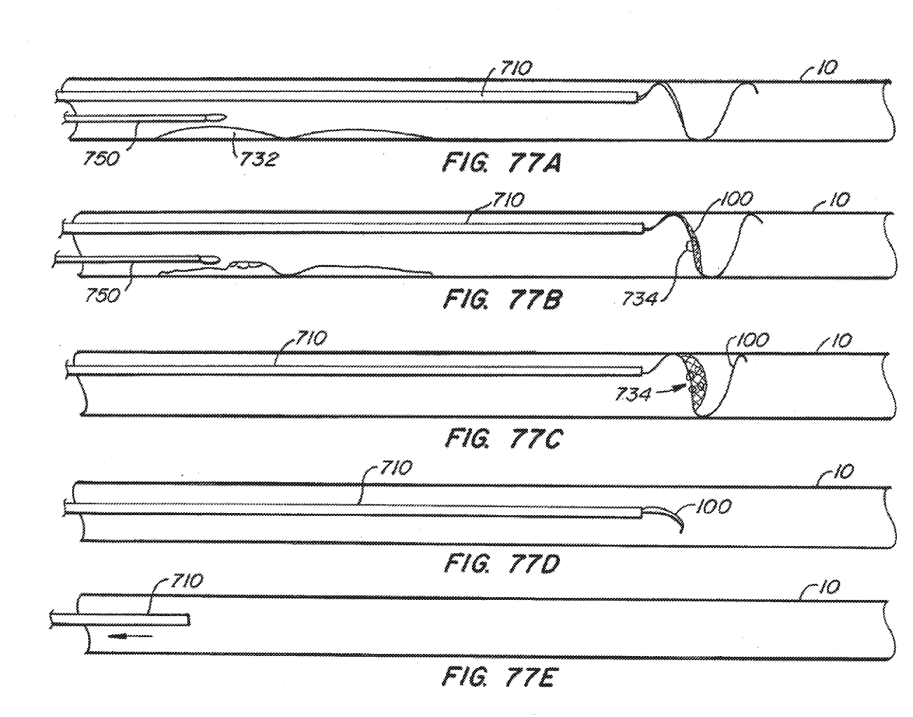

Embodiments of filter devices of the present invention may be used in methods of providing distal protection in procedures such as, for example, thrombectomy, arthrectomy, stenting, angioplasty and stent grafting. It is to be appreciated that embodiments of filter devices of the present invention may be used in veins and arteries. An exemplary procedure is illustrated in FIGS. 75A-I and FIGS. 76A-E. In each procedure, the device 100 is positioned in an un-tethered fashion adjacent to the treatment region 730. The sequence FIGS. 75A-I illustrate the delivery sheath 710 positioning FIG. 75A, complete deployment FIG. 75B into the lumen 10. A conventional treatment device 750 using mechanical, electrical energy or other suitable method is used to clear the undesired material 732 from the lumen wall (FIG. 75C). Some debris 734 removed from the lumen wall through the use of treatment device 750 is subsequently embolized into the blood stream (FIG. 75C) and trapped by the filter 100 (FIG. 75D). The conventional treatment device 750 is removed (FIG. 75E) and thereafter the advancement of recapture sheath 710 is advanced into recovery position (FIG. 75F).

The entrapped debris 734 is then removed prior to recapturing the device with methods such as, for example, aspiration, delivery of therapeutic agents or maceration. Additionally, the device and entrapped debris can be recaptured in whole and removed via the same sheath used to recapture the device as illustrated in FIG. 75G. The device 100 and debris 734 are then withdrawn into the sheath 710 (FIG. 75H), and the sheath withdrawn from the vasculature (FIG. 75I).

Similarly, an additional use of the invention as un-tethered distal protection is illustrated in FIGS. 76A-E, in which a balloon 751 is used to expand the lesion 732 such as in the case of balloon angioplasty, often performed prior to stenting a vessel to keep it open. For this procedure a balloon catheter is advanced to the lesion site and inflated FIG. 76 B, plaque 732 is pushed outward by the balloon (FIG. 76C), thus reestablishing normal blood flow. Any particulate matter 734 embolized by the procedure is trapped by the filter (FIG. 76D). The debris 734 can then be removed prior to filter retrieval as previously described or the device with trapped debris can be removed together (FIG. 76E).

An additional method practiced widely in the art is the use of tethered distal protection adjunctive to the previously described procedures (i.e., the device 100 remains tethered during the procedure). Embodiments of the filtering device of the present invention may also be used for this purpose as illustrated in FIGS. 77A-77E. Positive control of the filter 100 is maintained via an integral wire or snare connected to the device 100. The connection between the integral wire or snare to the device 100 is maintained during the procedure and may be, in some embodiments, used as a guidewire. As illustrated in FIG. 77B, connection to the device 100 is maintained a while performing a procedure to treat the vasculature in proximity to the location (i.e., treat the lesion 732).

An example of a tethered distal protection method is illustrated in FIGS. 77A-77E. An embodiment of a filter device 100 is deployed distal to the lesion 732 to be treated (FIG. 77A), the treatment is initiated (FIG. 77B), and embolized material 734 is captured in the filter 100 (FIG. 77C). Thereafter, the debris 734 is removed prior to filter recapture or, alternatively, with treatment in the filter 100 via a sheath as previously described. The device 100 is recovered into the sheath (FIG. 77D) and removed from the lumen 10 (FIG. 77E).

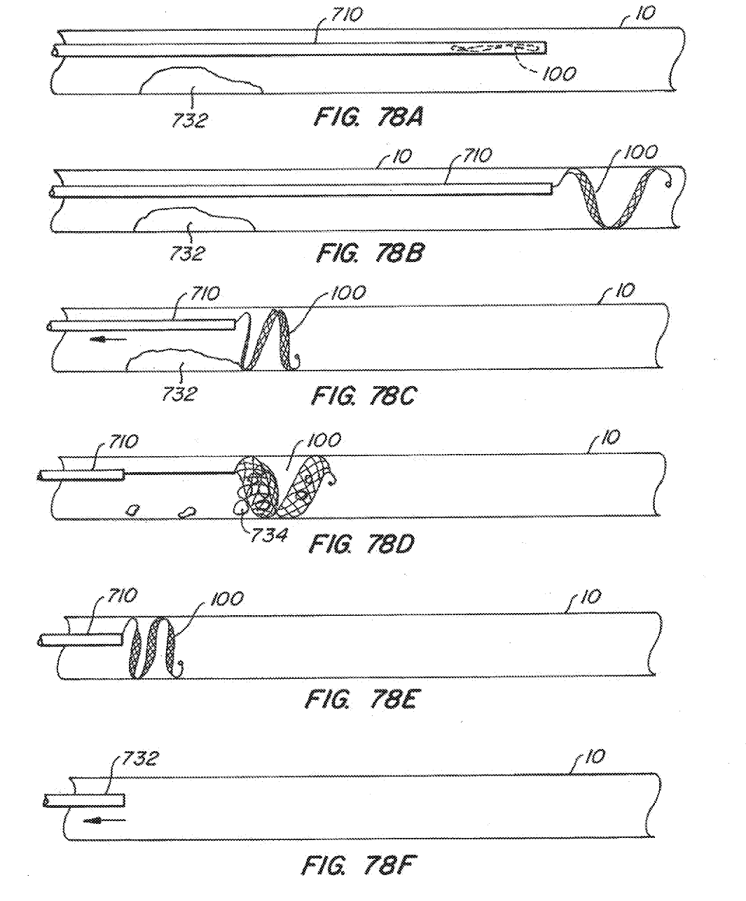

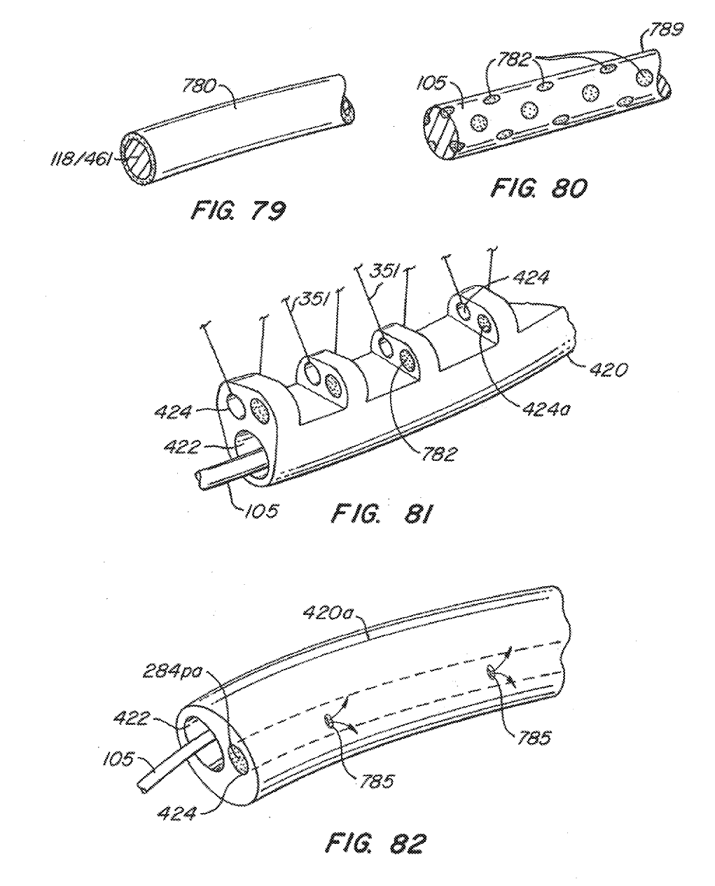

A tethered device (FIG. 77A, 78A) can also be employed to mechanically dislodge and remove embolic material 732 from a vessel 10, such as in the case of a thrombectomy. This offers a simple means of removing and trapping debris without requiring multiple devices to achieve the same goal. For this method, the tethered device is advanced downstream of the lesion site (FIG. 78A), and deployed (FIG. 78B). The tethered, deployed filter 100 is then drawn across the lesion 732 (FIG. 78C) to pull the thrombus from the vessel wall and into the filter 100 (FIG. 78D). The embolized material 734 is then removed via the methods previously described (FIG. 78E), tethered device is drawn into the sheath and removed from the lumen (FIG. 78F).

### Delivery of Pharmacological Agents Using Filtering Devices

Embodiments of the filter device of the present invention may also be used for delivering a pharmacological agent within a lumen. Delivery of a pharmacological agent within a lumen may be accomplished using any component of the filtering device. For example, the filter support structure may deliver a pharmacological agent. In one alternative, the support structure is covered by a multi-lumen structure and the multi-lumen structure is configured to release a pharmacological agent. In one alternative, a lumen of the multi-lumen structure is at least partially filled with a pharmacological agent. In another aspect, a lumen in a multi-lumen structure has ports that allow for the release of a pharmacological agent stored within the lumen. In one alternative, a cavity formed in a support member is filled with a material. In one aspect, the material in the cavity is a pharmacological agent. The filter may deliver a pharmacological agent. In one aspect the material capture structure is coated with a pharmacological agent.

Additional embodiments of the invention provide for the ability to deliver therapeutic agents via the material capture structure as well as the support structure covering. FIG. 79 illustrates a therapeutic agent coating 780 attached to a filament 118/461. FIG. 80 illustrates a composite structure 789 formed by having one or more cavities formed in a support structure 105 filled with one or more therapeutic agents or other material. The cavities may be formed as described above with regard to FIGS. 33, 35 and 36. These composite structures can be designed to elute a therapeutic agent via a specific elution curve by varying thickness, density as well as location of the therapeutic agent on the filter device component. This therapeutic agent could be, for example, any pharmacological agent used in the treatment of the body, an anti-coagulant coating (i.e., Heparin), an anti-proliferative agent prevent or slow fibrous tissue growth, other agents selected from those used in vascular stents including drug eluting stents.

FIG. 81 and FIG. 82 illustrate the use of the covering 420, 420a positioned over a support structure as the delivery means for providing pharmacological agents into a lumen. FIG. 81 illustrates a pharmacological agent 782 in a lumen 424a of a multi-lumen structure such as described above with regard to FIGS. 44, 45. As illustrated in FIG. 82, the therapeutic agent 784 fills a lumen 424 in a multi-lumen covering 420a over the support structure 105. Release ports 785 formed in the side of lumen 424 allow delivery of the agent to the blood or tissue. Control of the therapeutic agent elution parameters could be controlled via the size or spacing of the release ports 785 and/or through the use of controlled release pharmacological agents.

### Prototype Filtering Devices

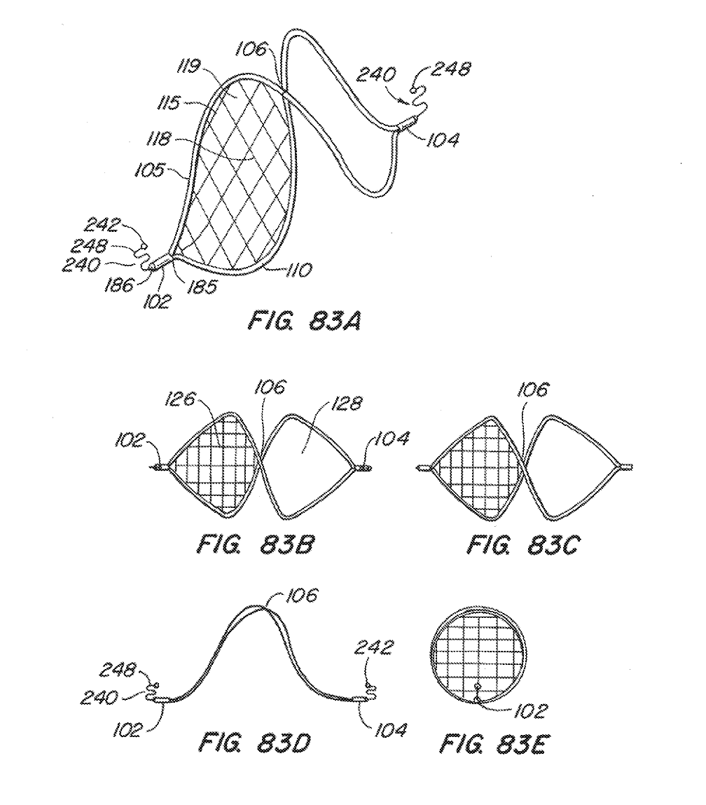

FIGS. 83A-83E illustrate perspective (FIG. 83A), plan (FIG. 83B), bottom (FIG. 83C), side (FIG. 83D) and end (FIG. 83E) views of a prototype filter according to an embodiment of the present invention. The prototype has previously described features and common elements have the same reference numbers have been incorporated into these illustrations. The support structure 105, 110 was formed with electropolished 0.015″ OD Nitinol wires, shape set to form two substantially equal open loops 126, 128 of approximately 1″ diameter. The support structure wire used for support structure 105 was ground down to a wire diameter of 0.010″ and used to form flexible retrieval feature 240 on each end (i.e., FIG. 28C). An atraumatic feature (here ball 242) is created on the end of the wire by exposing the wire to plasma. A radio opaque marker, here a Tantalum marker band 248 attached below the ball 242. The material capture structure 115 has filter cells 119 constructed with filaments 118. The filaments 118 are ePTFE monofilament. The filaments are attached to the support structure using method shown in FIG. 47. The cover 185 used to join the ends is a tapered Nitinol tube 186 that is crimped around the support structures, as illustrated in FIG. 24.

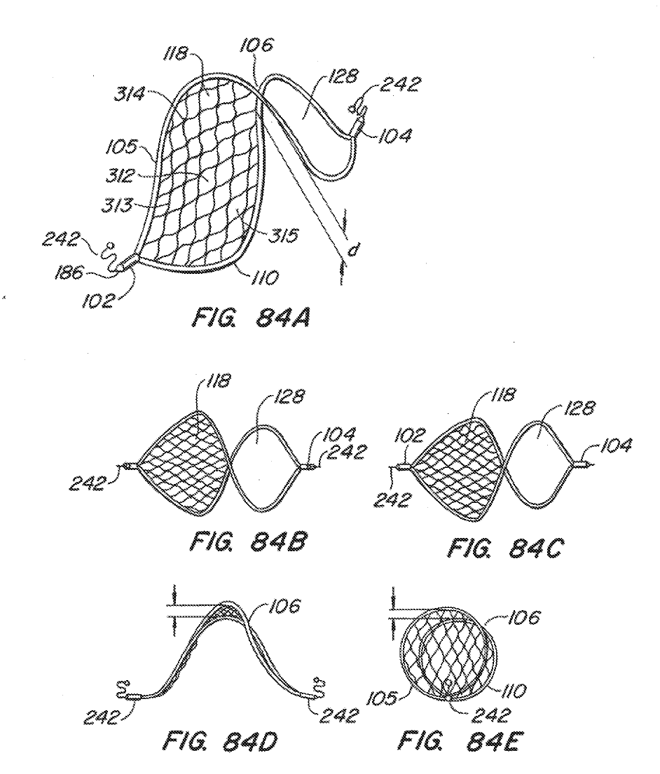

FIGS. 84A-84E illustrate perspective (FIG. 84A), plan (FIG. 84B), bottom (FIG. 84C), side (FIG. 84D) and end (FIG. 84E) views of a prototype filter according to an embodiment of the present invention. This embodiment is similar to the embodiment of FIG. 83A. In this embodiment, the material capture structure 115 is replaced with material capture structure 312 made of an extruded polymeric netting as described above with regard to FIG. 56. This embodiment also illustrates how the support structures 105, 110 are not in contact (i.e., separated by a distance “d”) at the crossover 106.

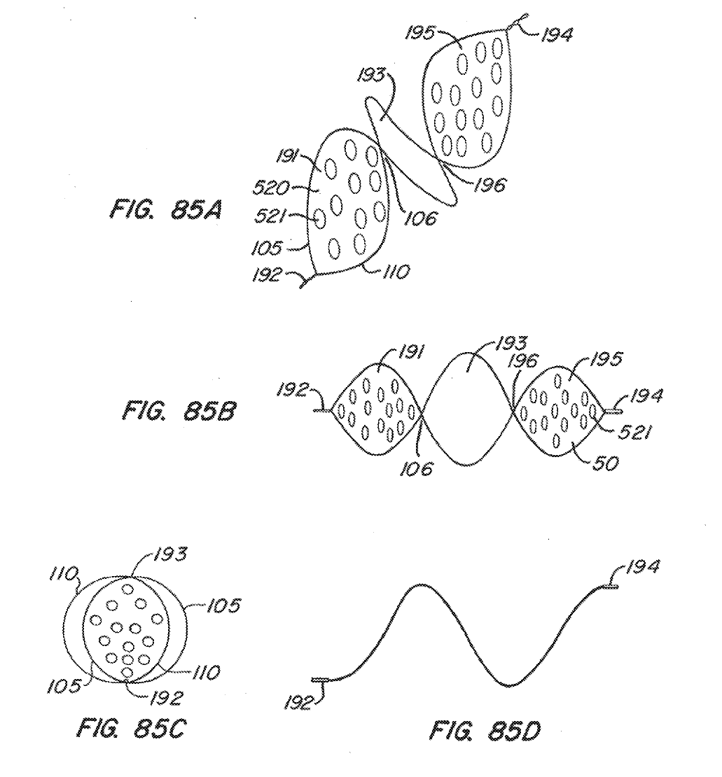

FIGS. 85A-85E illustrate perspective (FIG. 85A), plan (FIG. 85B), side (FIG. 85D) and end (FIG. 85C) views of a prototype filter according to an embodiment of the present invention. This embodiment is similar to the filter device described in FIG. 14A and common reference numbers are used. In this embodiment, a material capture structure is constructed from a continuous sheet of polymeric material 520 into which circular holes 521 are created via mechanical or laser cutting (as described above with regard to FIG. 61A).

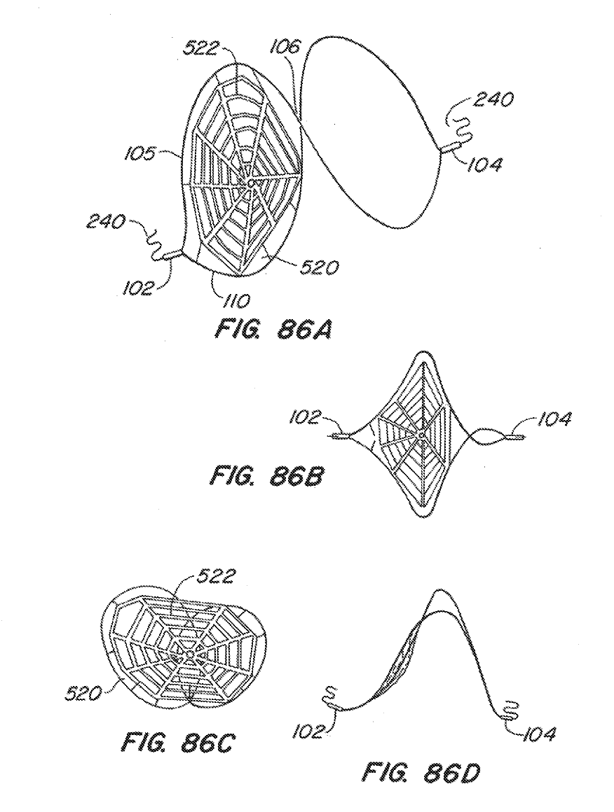

FIGS. 86A-86D illustrate perspective (FIG. 86A), plan (FIG. 86B), side (FIG. 86D) and end (FIG. 85C) views of a prototype filter according to another embodiment of the present invention. In this prototype filter, a material capture structure constructed from a continuous sheet of polymeric material 520 into which a pattern 522 voids are created via mechanical or laser cutting to create a net-like structure (FIG. 61C).

FIG. 87 is a perspective view of a prototype filter according to an embodiment of the present invention similar to the embodiment described in FIGS. 83A-83E above. In this embodiment the elongate structural members 105, 110 are joined at only one end (i.e., end 102). The support structure elements on the unconnected end are finished with plasma balls 242 to prevent vessel perforation and facilitate deployment and retrieval.

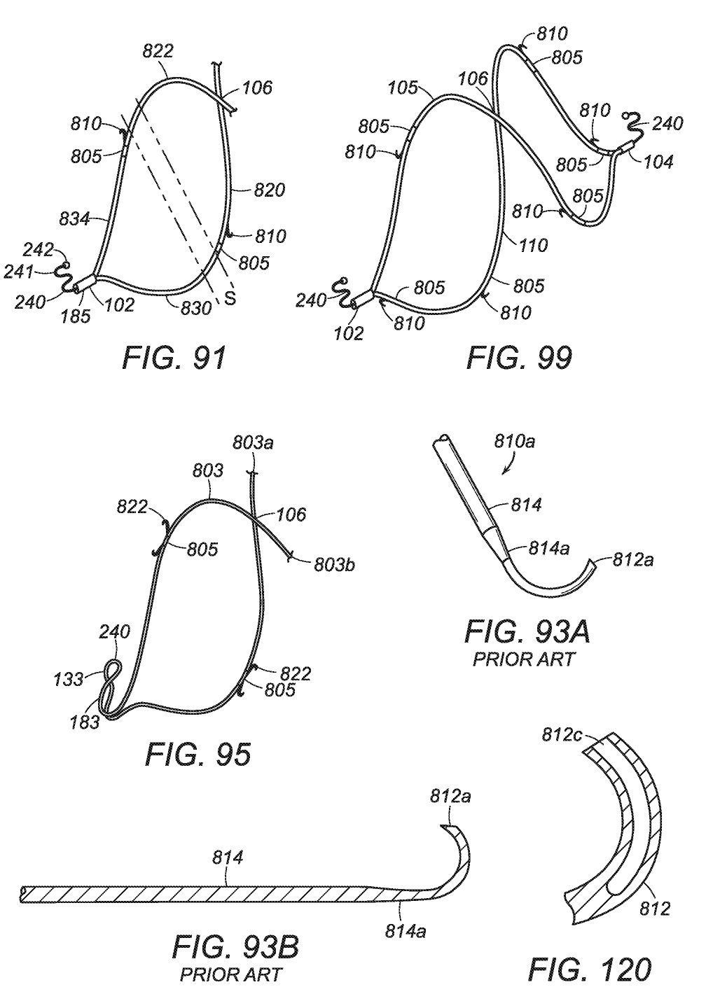

Some filter embodiments may include one or more fixation elements, tissue anchors or tissue engagement structures to aid in maintaining the position of the filter once deployed. The various alternative fixation elements, tissue anchors or tissue engagement structures are described below and may be adapted into a variety of combinations and configurations. FIG. 88 is a perspective view of an endoluminal filter having a first support member 105 having a first end and a second end and a second support member 110 attached to the first end of the first support member 105 or the second end of the first support member 105. In the illustrated embodiment, the first support member 105 and the second support member 110 are each formed from a single wire that extends from at least the first end 102 to the second end 104. The support members may extend beyond the end 102, 104 and be used to form retrieval features 240 or other elements of the filter as described below. In one illustrative example, the first support member 105 may be formed into a tissue anchor and the second support member 105 may be formed into a retrieval feature. The illustrative embodiment has a retrieval feature 240 on the first end 102 and a retrieval feature 240 on the second end 104. The second support member 110 forms a crossover 106 with the first support member 105. In one embodiment, the second support member 110 is attached to the first end of the first support member 102 and the second end of the first support member 104. A material capture structure 115 extends between the first and second support members 105, 110, the crossover 106 the first end or the second end of the first support member 105. In the illustrated embodiment, the material capture structure extends between the first and second support structures 105, 110, the first end 102 and the cross over 106. At least one tissue anchor 810 is on the first support member 105 or the second support member 110. In the illustrated embodiment, tissue anchors are provided on body supports 105, 110. In this embodiment, the fixation element 810 is a separate structure having a body 814 and a tip 812 suited for penetrating into or through the walls of lumen 10. The fixation element or tissue anchor 810 is attached to the elongate body using a suitable attachment 805. The attachment 805 may be a crimp (as illustrated) or any other suitable technique for joining the fixation element 810 to the elongate body. Suitable techniques include, by way of non-limiting example, a crimp or other joining technique with a discrete detent, a swage or other joining technique with circumferential constriction, soldering, welding, brazing, shrink fit tubing, epoxy, multi-lumen collar where one wire is placed in each lumen and then bonded or melted together. FIGS. 91 and 99 also illustrate possible configurations for filter structures formed from two elongate support members that are joined at the ends.

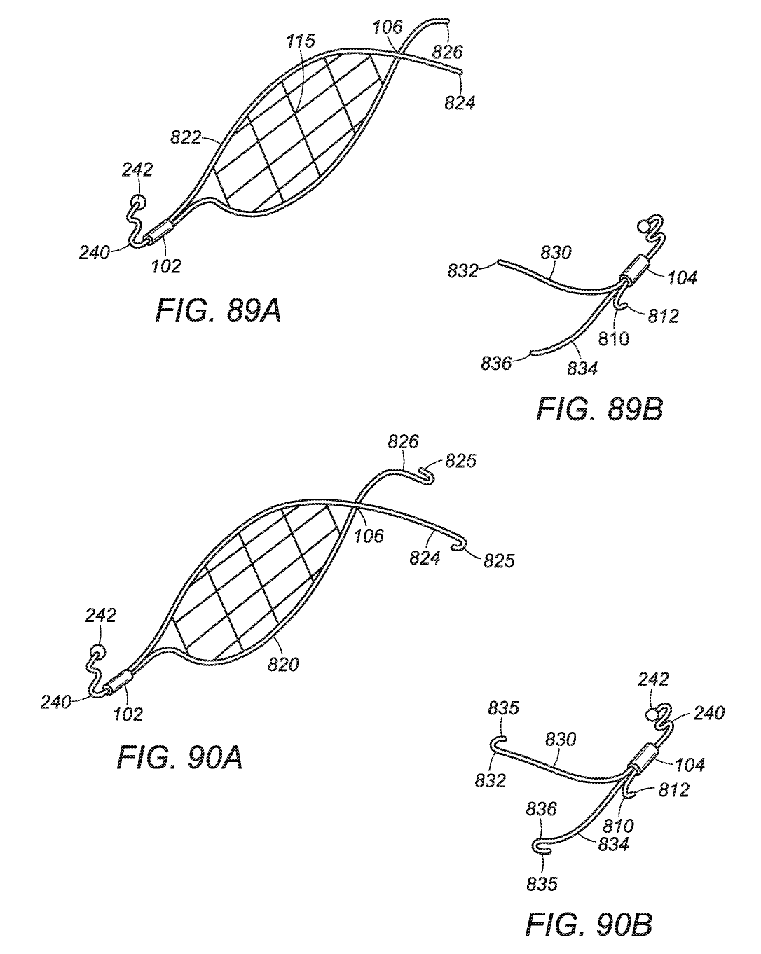

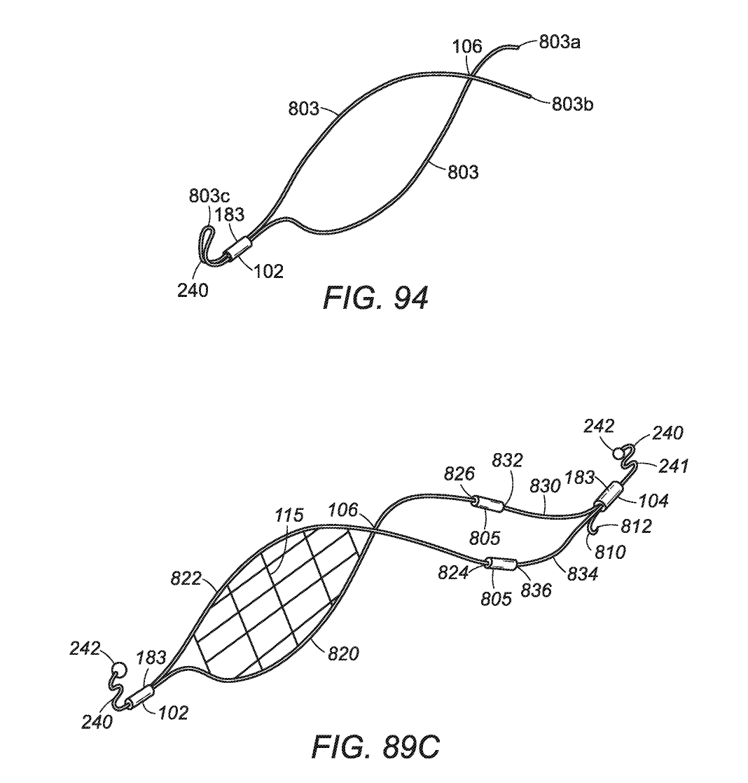

FIGS. 89A and 89B illustrate individual filter components that may be assembled into the final version illustrated in FIG. 89C. FIG. 89A illustrates the proximal end of the filter. The elongate bodies 820, 822 are used to secure a filter structure 115 between a cross over 106 and the end 102. The elongate bodies 820, 822 extend some length beyond the crossover 106 to ends 826, 824. A retrieval feature 240 is attached to end 240 and may be formed, in one exemplary embodiment; from either elongate body 820, 822. FIG. 89B illustrates the distal end of the filter. The distal end of the filter is formed by elongate bodies 834, 830 joined by end 104. The length of elongate bodies 830, 834 may be adjusted to join with the elongate bodies 820, 822 in FIG. 89A to form an appropriately sized filter. The distal end also includes a retrieval feature 240 and a fixation element 810. The final assembled filter is illustrated in FIG. 89C where the proximal and distal filter ends are joined at suitable joining connectors 805. It is believed that the manufacturing procedure used for constructing a filter is simplified through the use of proximal and distal ends. Each of the ends may be fabricated separately in relatively fewer and easier steps than when fabricating a filter from two elongate bodies of nearly equal length as described elsewhere in this application. Additionally, suitable joining connectors 805 used to couple the proximal and distal ends may also be used to attach a fixation element to the filter frame as illustrated, for example, in FIG. 91, 95 or 99.

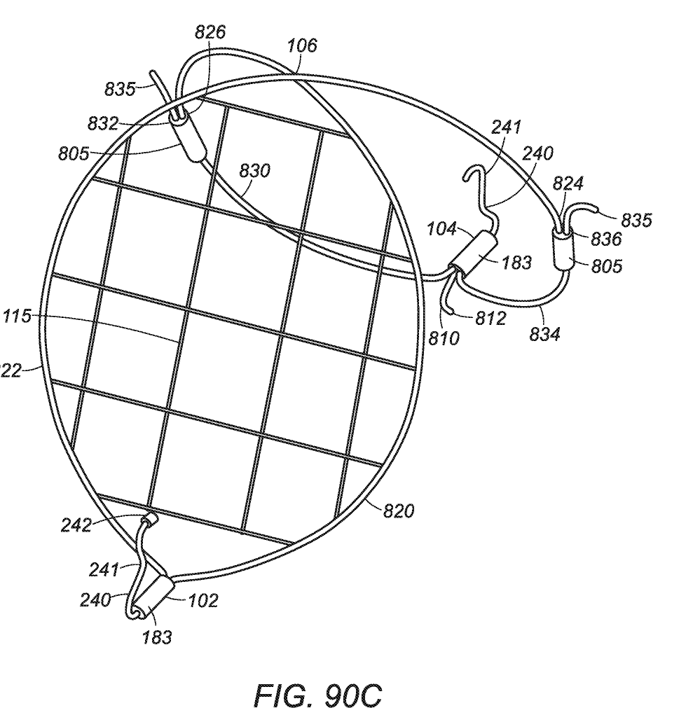

Alternatively, the ends of the elongate bodies could be used to form the fixation elements. FIGS. 90A and 90B illustrate proximal and distal filter ends with the tips of the elongate members modified to form fixation elements. The proximal filter end embodiment illustrated in FIG. 90A has hooks 825 formed on ends 824, 826. The distal filter end embodiment illustrated in FIG. 90B has hooks 835 formed on ends 832, 836.

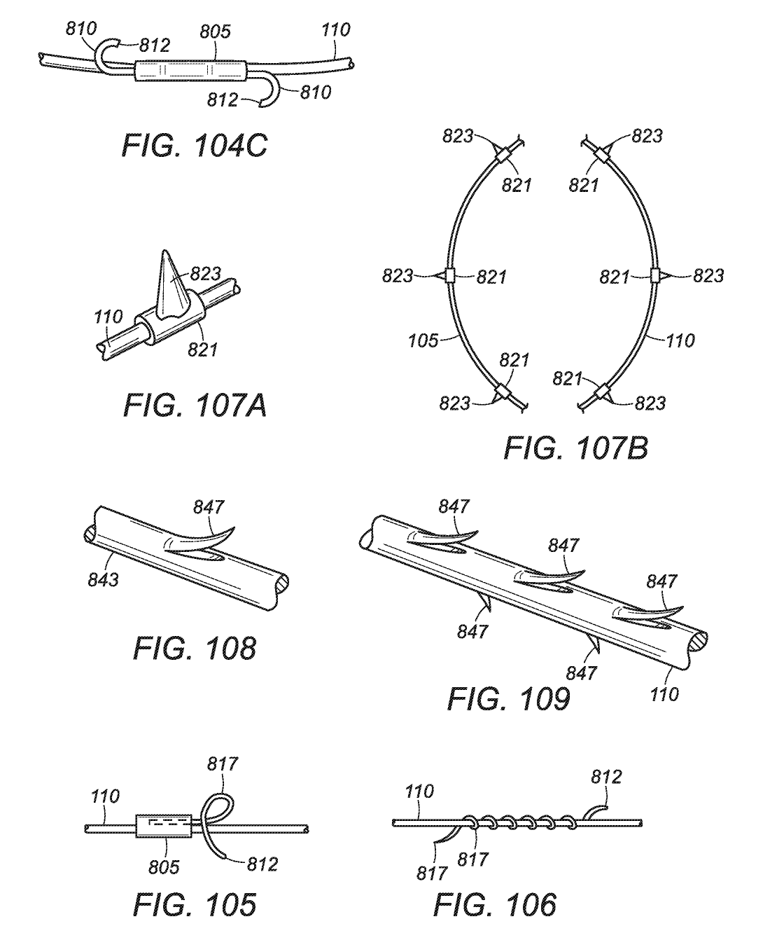

FIGS. 90A and 90B may be combined using a suitable joining connector(s) 805 to form a double hook fixation element such as illustrated in FIGS. 95, 104A, 104B, and 104C. Alternatively, the modified distal and proximal ends in FIGS. 90A and 90B may be combined in any combination to the unmodified distal and proximal filter ends illustrated in FIGS. 89A and 89B. FIG. 90C illustrates an embodiment of one combination that joins the proximal end in FIG. 89A with the distal end in FIG. 90B. Other combinations are possible. For example, the tissue anchor is on the first or the second attachment means. Additionally or alternatively, there can be a retrieval feature on the end of the first support structure and a retrieval feature on the end of the second support structure.

These, along with other embodiments, illustrate a filter support structure having a first support member having an end, a first segment extending from the end and a second segment extending from the end. There is also a second support member having an end and a first segment extending from the end and a second segment extending from the end and crossing but not attaching to the first segment. There is a first attachment means for joining the first segment of the first support member to the first segment of the second support member and a second attachment means for joining the second segment of the first support member to the second segment of the second support member. A tissue anchor is provided on or with the first or the second support member. As described in further detail above, there is also a material capture structure attached to the first and second segments of the second support member and between the end of the second support member and the place where the first segment crosses the second segment.

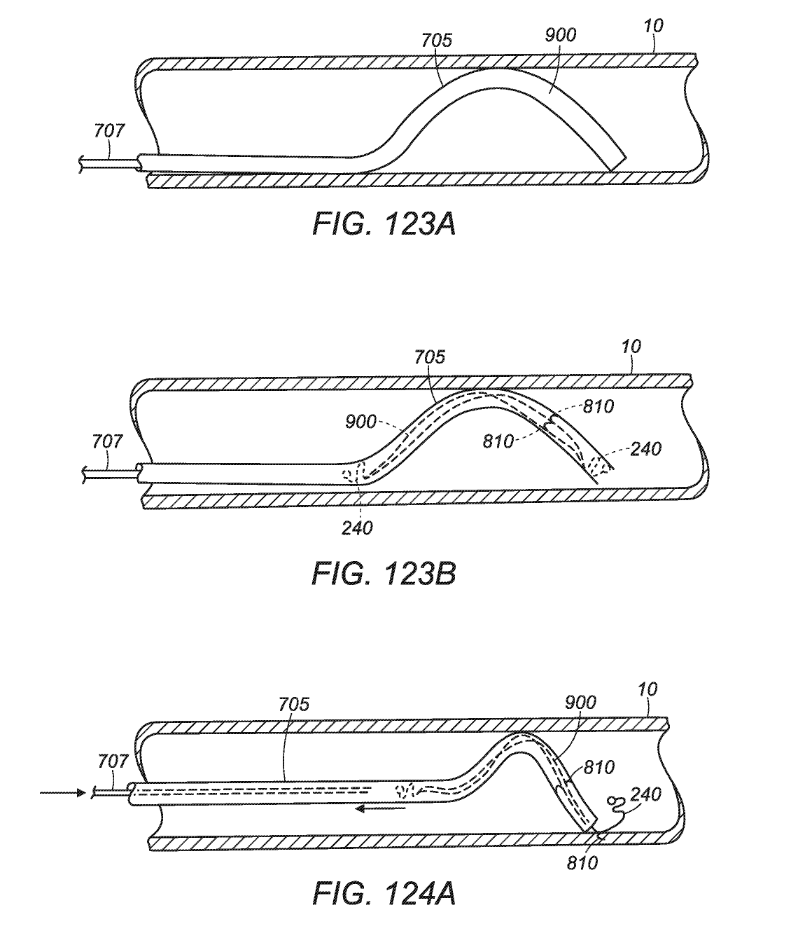

Additionally, while FIGS. 89A-90B illustrate elongate body components having the same or nearly the same length, the design is not so limited. The use of elongate bodies of different length can be used to position the fixation elements in off set locations along the elongate body. The elongate body lengths 820, 822, 830, 834 may be of different lengths than in previous examples attached as shown in FIG. 91. The use of different elongate body lengths produces a spacing (indicated by “s” in the figure) between the attachments 805. The dashed lines indicate the position of each fixation element when the fixation elements are moved into a stowed condition. The offset spacing “s” reduces the likelihood that the fixation element 810 between elongate bodies 820, 830 will become entangled with the fixation element 810 between elongate bodies 822, 834 when the filter is stowed prior to delivery (see FIG. 123B). Alternatively or additionally, the offset spacing “s” may be achieved by placing fixation elements on the elongate bodies in positions that result in the desired amount of offset to prevent the fixation elements from getting tangled.

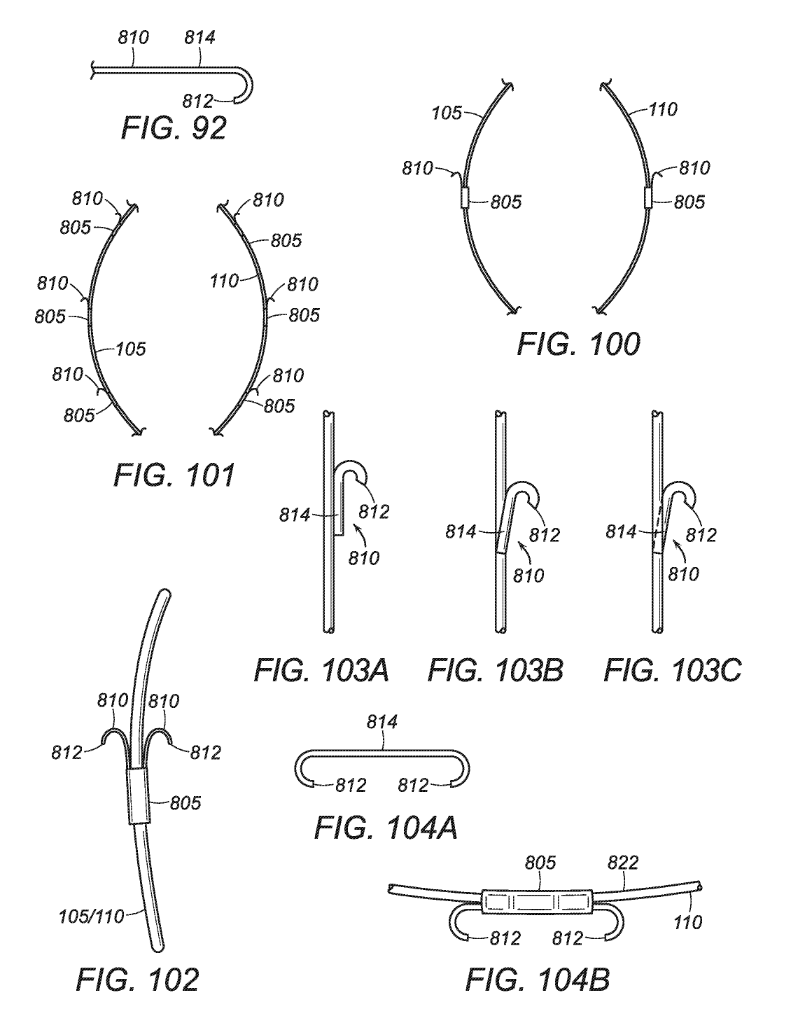

There are a number of various fixation elements that may be used. The fixation elements 810 shown in FIG. 92 illustrates a fixation elements that may be formed on the ends of the elongate bodies (i.e., FIGS. 90A and 90B) using a number of bending and forming techniques. The end may remain at the same diameter as the rest of the elongate body as shown in FIG. 92. The end is shaped into the desired curve between the body 814 and the tip 812 for engagement with the surrounding lumen. In one alternative embodiment, the elongate body end is cut, ground or otherwise shaped into a sharpened point or beveled tip 812. Additionally or alternatively, the fixation element may have a smaller diameter than the remainder of the elongate body as illustrated in FIGS. 93A and 93B. Fixation element 810a has an elongate body diameter that is reduced in a transition section 814a down to the desired final diameter of the tip 812a. The now reduced diameter end is then shaped into the desired curvature depending upon how the fixation element is to engage with the surrounding tissue. In an alternative embodiment, the transition section 814a alone or in combination with the tip 812a may be formed from a different material that the body 814. The difference in the materials or different qualities of the same material may be used to provide a barb or tissue anchor with a flexible tip. For example, either or both the transition 814a and the tip 812a may be formed from a flexible biocompatible material such as polytetrafluoroethylene (ePTFE), polytetrafluoroethylene (PTFE), Poly(ethylene terephthalate) (PET), Polyvinylidene fluoride (PVDF), tetrafluoroethylene-co-hexafluoropropylene (FEP), or poly(fluoroalkoxy) (PFA), other suitable medical grade polymers, other biocompatible polymers and the like.

FIG. 94 illustrates an embodiment of a proximal end 102 of a filter structure that is formed from a single wire 803. The wire 803 begins at the end 803a is curved into one side of the support frame and then into the retrieval feature 240. The wire 803 is reversed 803c to form the other side of the retrieval feature 240 and then the other side of the support frame to the end 803b. A crimp 183 or other suitable fastener is used to maintain the shape and position of the retrieval feature 240. While this illustrative embodiment describes a single wire formation technique for a proximal end 102, this technique may also be applied to the formation of a distal end 104. The retrieval feature 240 may also take shapes other than the one in the illustrated embodiment and may, for example, be formed to resemble retrieval features illustrated in FIGS. 20-22 and 25-28C. As shown in FIG. 95, the single wire 803 may also used to form a loop 833 on the distal end 240. This illustrates a technique for forming both the first support member and the second support member from a single wire. This embodiment also shows the connector 183 in a position raised above the lumen wall. Additionally, a double ended fixation element 822 is shown. This is an example of a tissue anchor having a first barb with a proximal opening and a second barb with a distal opening. The double ended fixation element may be formed by curving the ends of proximal and distal ends (see FIGS. 90A, 90B). Alternatively as shown in FIG. 104A, the fixation element 822 may be a stand alone component with a body 814 curved into two tips 812. As shown in FIG. 104B, the fixation element 822 may be joined to any elongate body using a suitable fixation 805. In the illustrated embodiment, the fixation element 822 is attached to an elongate body 110. The ends 812 may also be curved in different directions or different angles as shown in FIG. 104C.

Any of a wide variety of bonding or joining techniques may be used to join the proximal and distal ends such as: soldering, welding, brazing, shrink fit tubing, epoxy, multi-lumen collar where one wire is placed in each lumen and then bonded or melted together, twisting wires together. Alternatively, one or more techniques could be used to join the elongate bodies with or without the addition of a fixation element. Then, in order to reduce surface defects to initiate tissue growth, the area where the joining occurred is covered by a smooth material. The joined area could be coated with an epoxy or medical grade silicone, or a shrink fit tube or slotted tube could be placed over the join and then melted into place. Consider FIGS. 89A and 89B in an illustrative example of an alternative technique to provide a smooth surface to a joined area. First, a segment of heat shrink tubing is sufficiently long to cover the length of the elongate body included in the joining process is placed on the elongate bodies 830, 834 over the ends 832, 836, respectively, of FIG. 89B. Next, the ends 832, 836 in FIG. 89B are joined to the ends 824, 826 in FIG. 89A. Thereafter, the heat shrink tubing segments are advanced over the joined area and heated. As the heat shrink tubing segment is heated, it melts around the joined area and provides a smooth surface that seals the area where the end 826 joins end 832 and end 824 joins end 836.

The joint 805 is an example of an attachment element that joins the first support member to the second support member. The joint 805 could be used to join elongate bodies together as suggested by the embodiments illustrated in FIGS. 88, 89A, 89B, 90A, 90B, 94 and 96. Alternatively, the joint could be used to secure a fixation element to the filter frame. In yet another alternative, the joint could provide means for both joining the elongate bodies together into a single frame as well as joining a fixation element to the filter frame at the same point that the elongate bodies are joined. Suitable means for attachment and attachment techniques used to create the joint 805 include, by way of non-limiting examples, a crimp or other joining technique with a discrete detent, a swage or other joining technique with circumferential constriction, soldering, welding, brazing, shrink fit tubing, epoxy, multi-lumen collar where one wire is placed in each lumen and then bonded or melted together.

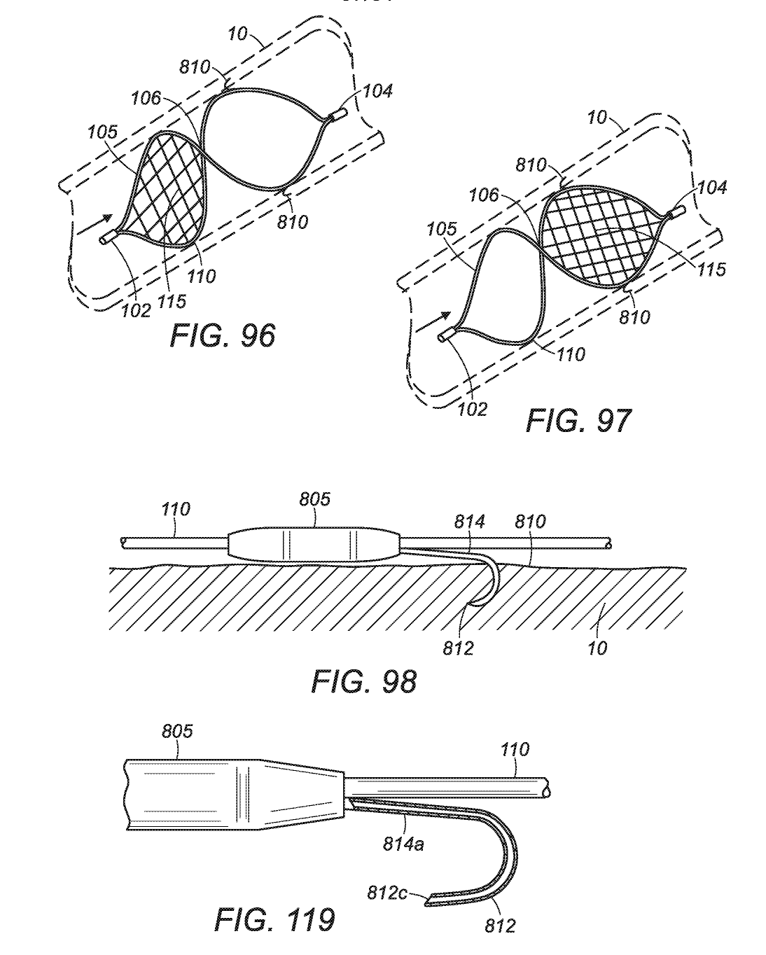

The material capture structure 115 may be in any of a number of different positions and orientations. FIG. 96 illustrates an embodiment of a filter of the present invention having two open loop support frames formed by support members 105, 110. Flow within the lumen 10 is indicated by the arrow. In this embodiment, the material capture structure 115 is placed in the upstream open loop support structure. In contrast, the material capture structure may be positioned in the downstream open loop support structure (FIG. 97). In another alternative configuration, both the upstream and the downstream support frames contain material capture structures 115.

There are filter device embodiments having equal numbers of support frames with capture structures as support frames without capture structures (e.g., FIGS. 13A, 13B, 96, and 97). There are other embodiments having more support frames without capture structures than there are support frames with capture structures. For example FIG. 14 illustrates a filter embodiment 190 having more support frames without capture structures than support frames with captures structures. The filter device 190 has two support members 105, 110 that are positioned adjacent to one another to form a plurality of support frames that are presented to the flow within the lumen 10. These support frames could also be modified to include fixation elements in any combination or configuration described herein. Alternatively, the plurality of support frames positioned to support a material capture structure across the flow axis of the device 190 or the lumen 10. The support members are joined together at end 192 and have two inflection points before being joined at end 194. The support members 105, 110 cross over one another at crossovers 106 and 196. The support frame 191 is between end 192 and crossover 106. The support frame 193 is between the crossovers 106, 196. The support frame 195 is between the cross over 196 and the end 194. One or more fixation elements may be provided in any or all of the support frames 191, 193 and 195 as described herein.

FIG. 98 illustrates a fixation element 810 engaged within the side wall of lumen 10. In this embodiment, the length and curvature of the fixation element is selected to remain within the wall of the lumen 10. As shown, the tip 812 is within the sidewall of lumen 10. In other alternative configurations, the length and curvature of a fixation element is selected engage with the lumen 10 by piercing though the lumen wall.

The fixation element could be a separate element or formed from one of the elongate bodies. Additionally, fixation elements may be positioned in any of a number of different positions and orientations. FIG. 88 illustrates fixation elements positioned about half way between an end 102, 104 and the cross over 106. An additional fixation element is positioned on the end 104. Unlike the illustrative embodiment of FIG. 88 where the fixation elements are on a single support frame, FIG. 99 illustrates the location of additional fixation elements on both support frames as well as the ends 104, 102. FIG. 99 does not illustrate any material capture structure within the frame. In FIG. 99, the fixation elements 810 are positioned along both elongate bodies 105, 110 about mid-way up on the support frame between an end and the crossover. Alternative fixation element 810 spacing and orientation is illustrated in FIGS. 100 and 101. FIG. 100 illustrates placement of the fixation elements 810 about mid-distance between the ends 102, 104 and the cross over 106. FIG. 101 illustrates the placement of the fixation elements similar to FIG. 100 with additional elements positioned near the cross over 106 and an end 104, 104. As illustrated in FIG. 102, more than one fixation element or barb may be positioned at each location along the structure. FIG. 102 illustrates a fixation attachment point 805 that secures two fixation elements 810 to the elongate body 105, 110. The fixation elements 810 may be provided separately or, alternatively, one or both of the fixation elements 810 may be formed from the elongate bodies. More than one barb or fixation element on a single location along the filter structure is also illustrated in FIGS. 95, 104A, 104B and 104C, for example.

Returning to FIG. 88, attachment portion 805 could also be used to mount or secure an individual fixation element 810 to an elongate body. FIGS. 103A, 103B and 103C for individual elements may be attached on (FIG. 103A) or on the sides (103A, 103B) to provide the desired orientation to the lumen wall as well as provide the desired device profile. Cover or joining structure 805 used to secure the fixation element to the elongate body has been removed to show detail.

Fixation elements may be designed to engage, pierce or otherwise attach to the lumen sidewall with more than one attachment point. FIG. 102 illustrates more than one fixation element 810 attached to an elongate body at a single attachment site or with a single cover or joint structure 805. FIG. 104A illustrates a double ended fixation element 822 having a body 814 with two fixation tips 812. FIG. 104B illustrates the double ended fixation element 822 attached to an elongate body 110. FIG. 104C illustrates how the tips 812 may be altered to adjust the manner by which the tips engage with the adjacent lumen wall. FIG. 104C illustrates one proximally opening tip 812 and one distal opening tip 812.

Different fixation element body orientation and fixation positions for the tips 812 are possible. In one embodiment, the tissue anchor comprises a coil wrapped around the first support member or the second support member and an end raised above the first support member or the second support member. An illustrative example of one such tissue engagement or anchor is illustrated in FIG. 105. FIG. 105 illustrates a curved wire 817 extending along and wrapped around the elongate body and then curling to place a curl between the fixation portion 105 and the tip 812. The degree of curvature of the curved wire 817 may be adjusted to control the force used to pierce the tissue or control the amount of fixation force applied to the lumen walls. Alternatively, as illustrated in FIG. 106, the fixation element body 817 may attach to the elongate body 110 by wrapping around a length of the elongate body. FIG. 105 also illustrates an example where the tissue anchor is a coil or open tube having a tissue engagement surface comprising a raised spiral form. FIG. 105 also illustrates a tissue anchor having an attachment section attached to the first support member or the second support member, an end adapted to pierce tissue and a coil 817 between the attachment section and the end 812. An optional covering (not shown) may also be placed over the coiled wire 817 to maintain a smooth device profile along the elongate body 110.

The filter structure may also be secured using alternative fixation elements illustrated in FIGS. 107A, 107B. In some embodiments, a tissue anchor or anchors are formed from or attached to a tube that is attached to the first support member or the second support member. FIGS. 107A and 107B illustrate a tube or support 821 adapted to fit over the elongate body 110. A feature 823 on the support 821 is used to engage with sidewall of the lumen. In the illustrated embodiment of FIG. 107A, the feature has a generally conical shape with a pointed tip, similar to a thorn. One of more of the supports 821 may be placed along the elongate body 810 as illustrated in FIG. 107B. Alternatively, the feature 823 may be formed from or as part of an integrated structure with the support 821. The feature 823 may be formed in a different shape than illustrated. The feature 823 may take the form of a circumferential rib, or a void/dentent. In another alternative embodiment, the support 821 is a continuous piece that extends along the length or most of the length of the elongate body 110 rather than in discrete segments 821 illustrated in FIG. 107B. The size, number and spacing of the feature 823 or features 823 may vary depending on application. For anchoring a material capture structure in the inferior vena cava, for example, a feature 823 may have a height of between about 0.5 mm to about 3 mm have spacing of about 0.1 mm to about 5 mm.

FIGS. 108 and 109 illustrate another alternative fixation element. In these alternative embodiments, a tissue anchor is formed from the first support member or the second support member (FIG. 109) or is attached to or formed from a structure or tube that is attached to the first support member or the second support member (FIG. 108). Additionally or alternatively, a tissue anchor can be formed from or attached to a tube that is attached to the first support member or the second support member. FIG. 108 illustrates a tissue anchor that is a tube 843 having a tissue engagement surface. In this illustrative embodiment, the tissue engagement surface includes triangular fixation elements 847. The triangular fixation elements 847 may be formed in the sidewall of a hollow tube 843 as shown in FIG. 108. Then, the hollow tube 843 is then placed over and secured to the elongate body 110. Suitable materials for tube 843 include, for example: Nitinol, stainless steel or previously described polymers and degradeable polymers. The cross section of the hollow tube 843 is illustrated as round but other cross sections are possible. In one embodiment, the cross section of the tube 843 is sized and shaped to conform to the size and cross section shape of the elongate body 110. Alternatively, instead of forming the triangular fixation element(s) 847 in a tube that is placed over the elongate body, the triangular fixation members 847 are formed in or using the surface of the elongate body 110 as shown in FIG. 109. In this illustrative embodiment, the tissue anchor(s) on the first or the second support member are formed from the first or the second support member. While the illustrated embodiments show fixation elements 847 having a generally triangular shape other shapes are possible. For example, the fixation elements 847 may be shaped as an elongate spike or in any other suitable shape for engaging the adjacent lumen or tissue.

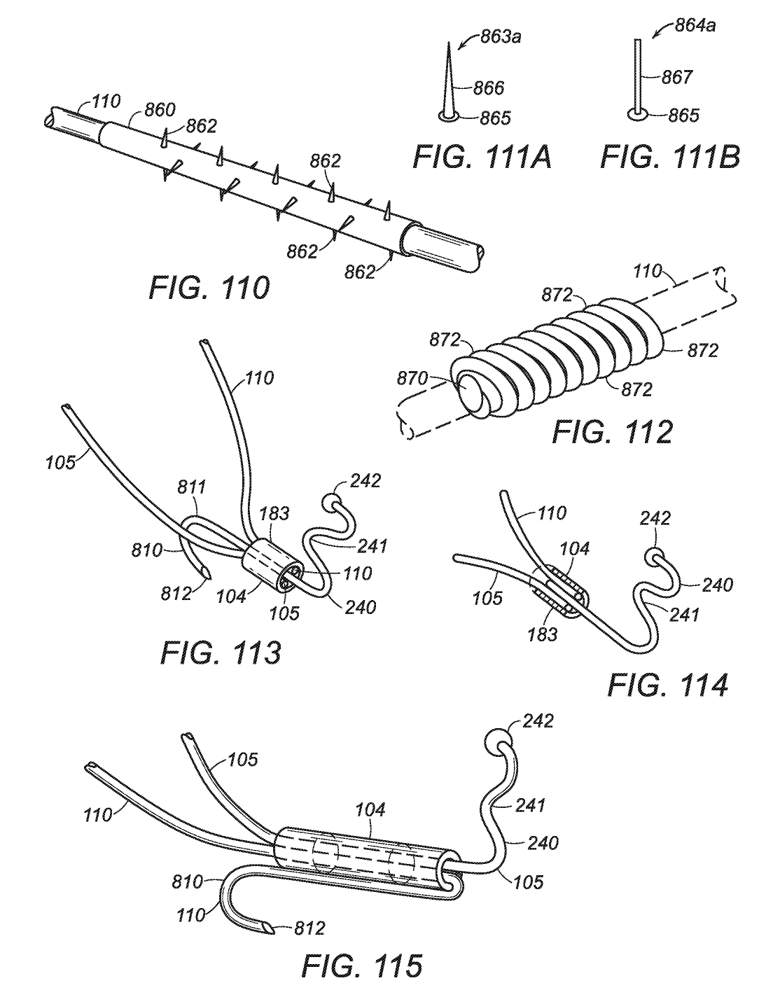

In another alternative embodiment illustrated in FIG. 110, the tube 847 may be modified to form a tissue engagement surface. In this illustrative embodiment, the tissue engagement surface includes surface features 862 that are shaped like spikes or thorns. One method of making the features 862 is to heat a polymer tube until the surface of the tube becomes tacky. Next, the surface of the tube is wicked up into the shape of the feature 862. As illustrated, the tube 860 is segmented to cover only a portion of the elongate body 110. In another embodiment, the tube is the same length or about the same length as the elongate body 110. Instead of modifying the surface of the tube 860, tissue engagement features 862 may instead be formed by mounting a fixation feature on, in or through the wall of the tube 860. A tissue engagement feature may take any of a number of different shapes as illustrated in FIGS. 111A and 111B. FIG. 111A illustrates a tissue engagement feature 863a with a base 865 supporting a sloped body 866 that ends in a pointed tip. FIG. 111B illustrates a tissue engagement feature 864a with a base 865 supporting a generally cylindrical body 867 that ends in a flat tip. The tissue engagement features may be added to the tube 860 by pushing them through the sidewall such that, when installed, the base 865 is within the lumen of the tube 860 and the body 866, 867 extends through the sidewall as shown in FIG. 110.

FIG. 112 includes another alternative embodiment of a tube based fixation element. In this embodiment, the tissue engagement surface comprises a raised form. In one embodiment, the tissue anchor is a tube having a tissue engagement surface comprising a raised spiral form. As shown in FIG. 112, the surface of the tube 870 has been modified into a raised spiral with ridges 872. The raised spiral 872 may be in a segment as shown. One or a plurality of segments may be attached along the length of the elongate body. Alternatively, instead of a segment, the tube 870 may be the same length as or about the same length as the elongate body 110 to which it is attached. In another alternative embodiment, the raised portion is formed by inserting a spring or other structure beneath the surface of the tube or segment 860. Additionally or alternatively, the tissue anchor comprises a coil wrapped around the first or the second support member. As illustrated in FIG. 106, this alternative can be formed by wrapping one wire (the elongate body 110) with another wire or a spring (wrapped wire 817). The wire 110 and wrapped wire 817 may then be coated by another material or placed into a suitable shrink tubing. Once the material or heat shrink is treated to conform to the wires, the resulting structure would resemble that shown in FIG. 112 with the addition that the tips 812 (see FIG. 106) would extend through the material to provide an additional attachment point to the lumen.

It is to be appreciated that the formation of tissue engagement structures may take any of a number of alternative forms alone or in any combination. As shown and described above in FIG. 109 features 847 may be cut into the surface of a elongate body. FIG. 108 illustrates how similar features may be cut into the walls of a tube 843. Additionally, the tissue engagement surface may take the form of a raised profile surface on the tube as shown in FIGS. 106, 112. Additionally or alternatively, the tissue engagement surface may be formed by roughening the surface of the tube or structure that engages the tissue, thereby increasing the coefficient of friction between the filter and the tissue it contacts. In some embodiments, the roughening may take the form of surface texturing by mechanical means (sanding, bead blasting knurling, cutting, scoring), chemical means (acid etching), laser cutting, or as an integral part of the extruding or molding process.

In addition to adding fixation or tissue engagement structures to the elongate bodies, the retrieval features may be attached to the elongate body or formed from the elongate body in a number of different ways that may also include a fixation element or elements. In one embodiment, there is a combined tissue anchor and retrieval feature joined to the first end or the second end of the first support member as shown in FIG. 113. FIG. 113 illustrates a distal end 104 where the elongate bodies 105, 110 terminate within the attachment element or securing feature 183. The securing feature may be a crimp 183 or any other suitable technique to join the elongate bodies together. Suitable means for attachment and attachment techniques used to create the attachment element or securing feature 183 include, by way of non-limiting examples, a crimp or other joining technique with a discrete detent, a swage or other joining technique with circumferential constriction, soldering, welding, brazing, shrink fit tubing, epoxy, multi-lumen collar where one wire is placed in each lumen and then bonded or melted together.

In this illustrative embodiment, the retrieval feature 240 is formed from a single wire 811 that is shaped into the curves 241 of the retrieval feature 240 as well as into a tissue engagement structure 810 having a tip 812 for engaging with tissue.

In this illustrative embodiment, the diameter of the wires used for the elongate bodies 105, 110 and the retrieval feature 240 are nearly the same so crimping the wires is suitable joining method. Other joining methods include, by way of non-limiting examples, a crimp or other joining technique with a discrete detent, a swage or other joining technique with circumferential constriction, soldering, welding, brazing, shrink fit tubing, epoxy, multi-lumen collar where one wire is placed in each lumen and then bonded or melted together.

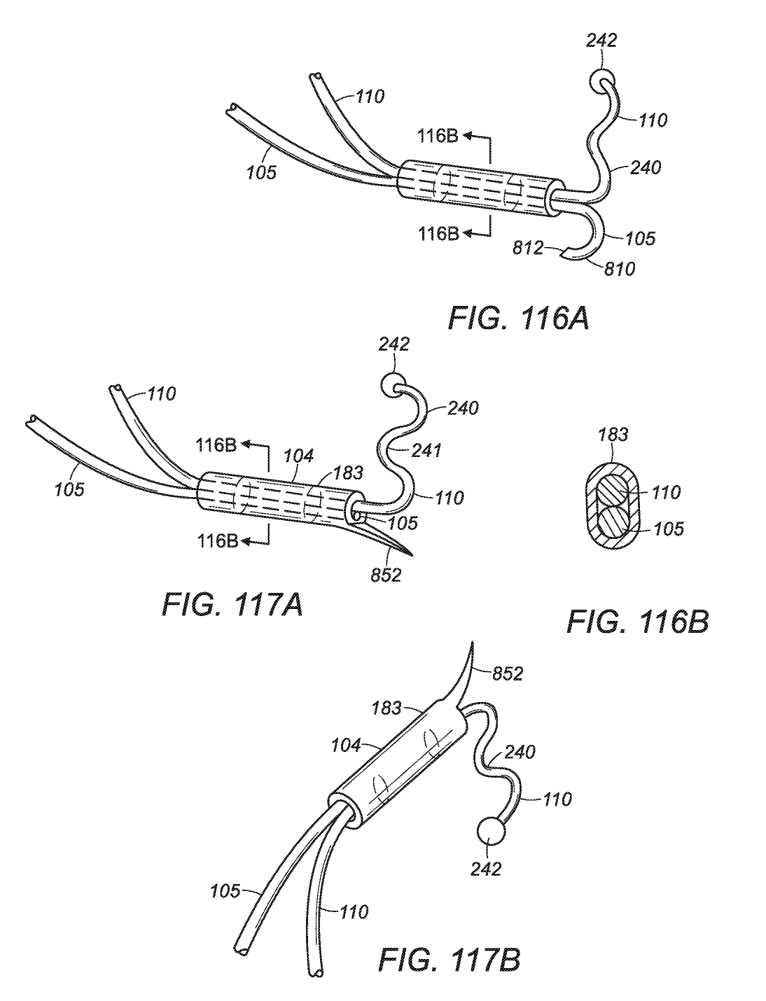

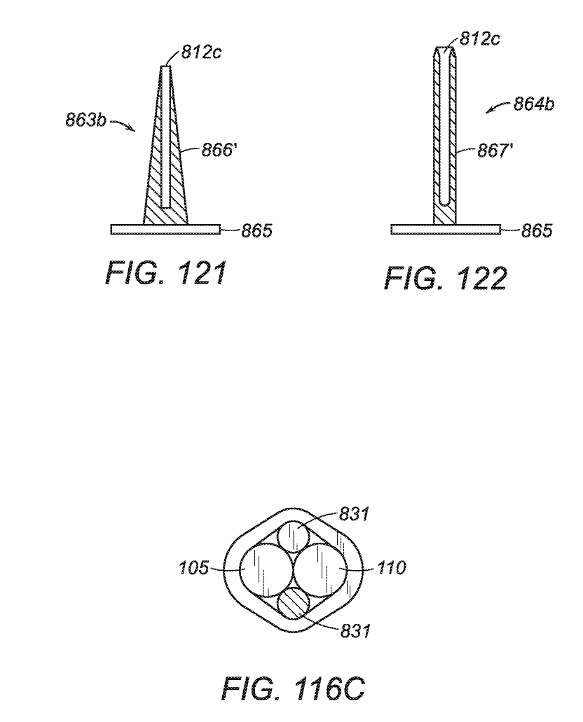

In the embodiment illustrated in FIG. 114, in contrast to the illustrated embodiment in FIG. 113, the wire used to form the retrieval feature 240 terminates within the securing feature or attachment element 183. Instead of using a separate wire as shown in FIGS. 113, 114, the ends of the elongate bodies 105, 110 may be used to form the retrieval feature 240 and a fixation element 810. This is an example of an end of the first support member forms a tissue anchor and an end of the second support member forms a retrieval feature. Additionally or alternatively, the retrieval feature formed on the end of the first support structure is formed from the first support structure or the retrieval feature formed on the end of the second support structure is formed from the second support structure. In some embodiments, a tissue anchor is on the end of the first support structure or the end of the second support structure. FIG. 115 illustrates the elongate body 105 passing through the crimp 183 and then being shaped into a retrieval feature 240. The elongate body 110 passes through the crimp 183 and then shaped into a distal opening fixation element 810 with tip 812. FIG. 116A is similar to FIG. 115 except that the elongate body 110 is used to form the retrieval feature 240 and the elongate body 105 passes through the crimp 183 and then being shaped into a proximal opening fixation element 810. FIG. 116B is a section view through the crimp 183. FIG. 116C is a section view of FIG. 116A with spacers 831 inserted into the crimp 183 to help distribute the crimp force and provide a more secure joint.

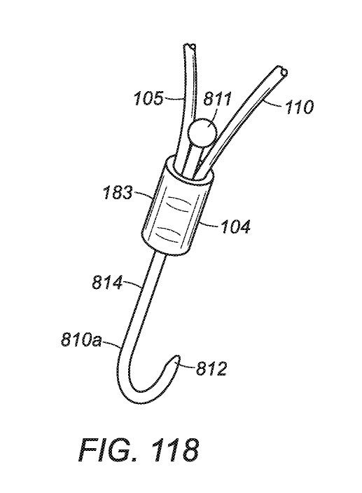

Instead of adding a fixation element to an end, the end may be used to form a fixation or tissue engagement element. FIGS. 117A and 117B illustrate perspective and bottom views where a fixation element 852 is formed from the crimp 183 used to hold the elongate bodies 105, 110. Either elongate body 105, 110 may be used to form the retrieval feature 240. FIG. 118 illustrates an alternative embodiment having a wire 814 separate from the elongate bodies 105, 110. The wire 814 is formed into a fixation element 810a where a ball 811 prevents the wire 814 from pulling through the crimp 183. The fixation element 810a ends in a hook 812.

The modifications a retrieval feature to include a fixation element as described with regard to FIGS. 88, 89B, 90B, 96, 99, 113, 114, 115, 116-118 may also be used to provide one or more fixation elements to the retrieval feature embodiments described with regard to FIGS. 20-29. Additionally, while many of the illustrative embodiments have been described in conjunction with elongate bodies 105, 110 the invention is not so limited. Other elongate body and/or support structures described herein may also be used interchangeably with the elongate bodies 105, 110.

In other alternative embodiments, all or a portion of the fixation element may be modified to include a pharmacological agent. The inclusion of a pharmacological agent may include coating all or a portion of the filter or tissue engagement structure with a pharmacological agent. Additionally or alternatively, the tissue engagement feature may be adapted and configured to contain a drug or combination of drugs or pharmacological agents that are released over time or after some initial time delay. FIG. 119 illustrates an alternative embodiment of the fixation element 812 in FIG. 98 with a hollowed end portion 812c. The drug eluting fixation element 814a may be formed using a hypodermic-like needle shaped into the desired curvature. Alternatively, the cavity 812c may be formed by hollowing out a portion of the interior a wire or by forming the fixation element 812 from a tube. Similarly, the tip of the fixation element in FIGS. 93A, 93B may be hollowed as shown in FIG. 120. FIG. 120 illustrates a cavity 812c in the distal end of the fixation element 812. The pins 867 and spikes 866 of FIGS. 110, 111A and 111B may also be modified to include a drug cavity as shown in FIGS. 121 and 122. FIG. 121 illustrates a tissue engagement feature 863b with a base 865 supporting a sloped body 866′ that ends in a pointed tip. A cavity 812c extends from the tip into the body 866′. FIG. 122 illustrates a tissue engagement feature 864b with a base 865 supporting a generally cylindrical body 867′ that ends in a flat tip. A cavity 812c extends from the flat tip into the body 867′. The cavities 812c may be filled with any of a wide variety of pharmacological agents. Examples include: anti-proliferative or anti-thrombogenic agents. Additionally, these or any other fixation element or tissue engagement structure embodiment may also be coated with a pharmacological agent.

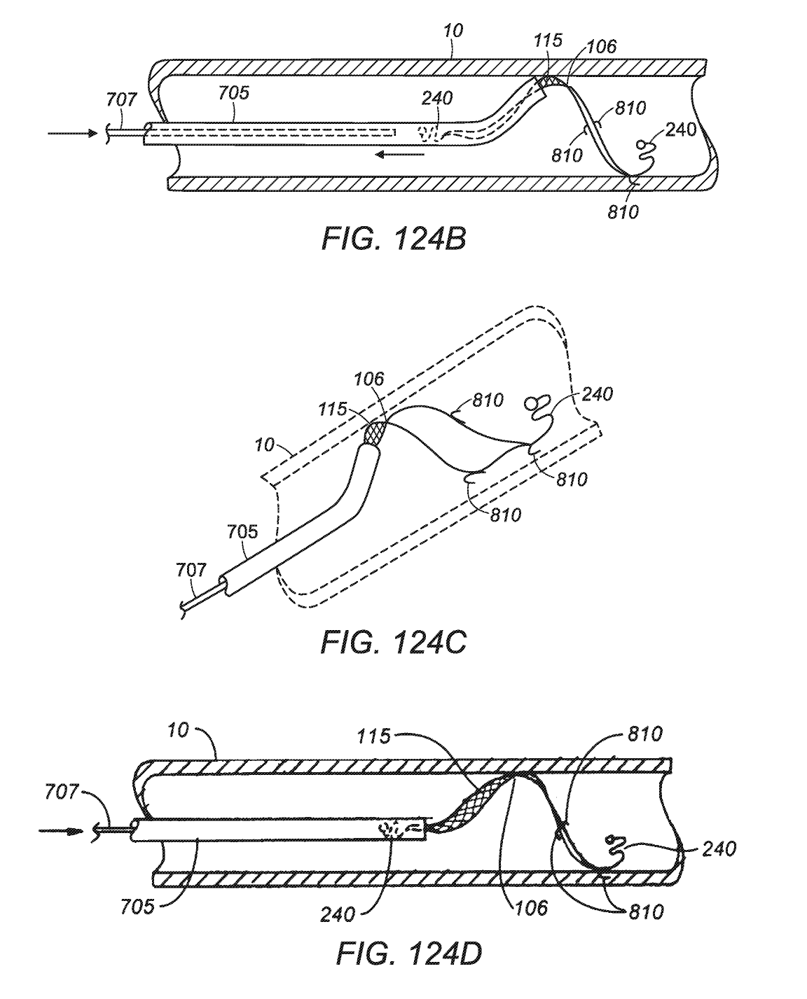

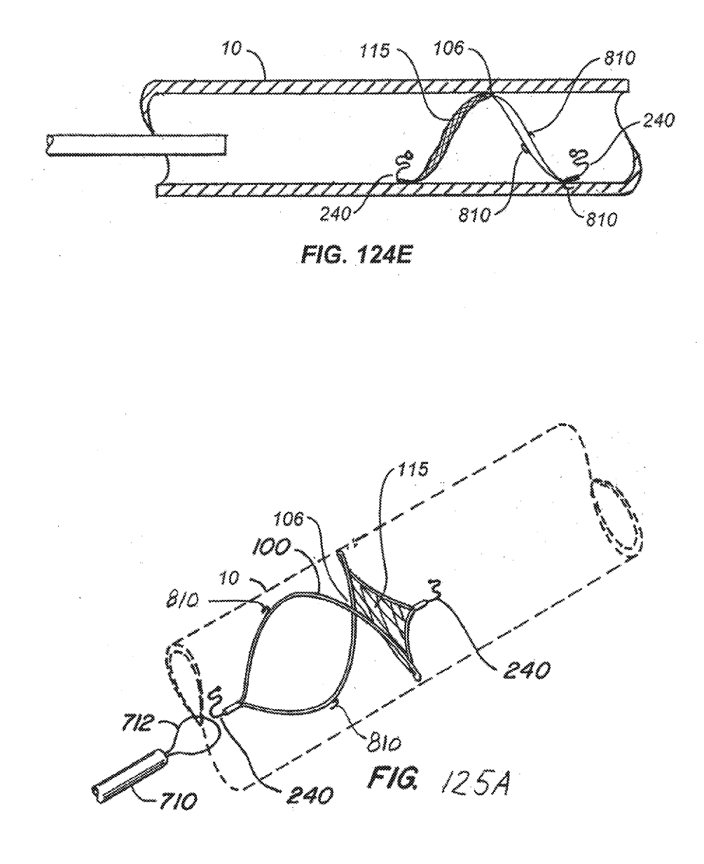

FIGS. 123A, 123B and 124A-E illustrate the positioning and deployment of an embodiment of the filter device 900 of the present invention having one or more fixation or tissue engagement features 810. The filter device 900 is an exemplary embodiment of any one of the alternative filter structure embodiments described herein having tissue engagement or fixation elements.

Embodiments of the present invention may be partially deployed so that a user may confirm the position of the filter prior to completely deploying the device into the target lumen. Partial deployment involves the controlled and reversible deployment and engagement of one or more fixation elements. The engagement is reversible because after placing the filter into the lumen the filter may be pulled partially or completely into the sheath as described herein. The filter may be repositioned and then redeployed into the lumen so that the fixation elements engage the lumen walls. Additionally, the design of embodiments of the filter of the present invention allow the retrieval action to be accomplished by approaching the filter from the same direction used for deployment. All the steps of positioning, deployment and recovery may be performed from a single access site.

The device 900 may be loaded into an intravascular delivery sheath 705 as shown in FIGS. 123A, 123B and as described above with regard to FIG. 69. Using conventional endoluminal and minimally invasive surgical techniques, the device 900 can be loaded into the proximal end of the sheath 705, before or after advancing the sheath 705 into the vasculature, and then advanced through the sheath using a conventional push rod. The push rod 707 is used to advance the device 900 through the delivery sheath 705 lumen as well as fix the position of the device (relative to the sheath 705) for device deployment. In one preferred technique, the device 900 is loaded into the proximal end of a delivery sheath that has already been advanced into a desired position within the vasculature (FIG. 123B). The device 900 may be pre-loaded into a short segment of polymeric tubing or other suitable cartridge that allows the device 900 to be more readily advanced through a hemostasis valve.

When used with a compliant delivery sheath 705, the pre-formed shape of the device 900 deforms the sheath 705 to conform to the device shape (FIG. 123A, 123B). Accordingly, a flexible, compliant sheath 705 assumes the curvature of the stowed device 900. The deformation of the delivery sheath 705 helps stabilize the position of the sheath 705 in the vasculature and facilitates accurate deployment of the device 900 to the intended delivery site. In contrast, a non-compliant delivery sheath 705 (i.e., a sheath that is not deformed to conform to the preformed shape of the device 900) maintains a generally cylindrical appearance even through the device 900 is stowed within it (FIG. 69C). Regardless of the type of sheath used, device delivery is accomplished by using the push rod 707 on the proximal side of the device 900 to fix the position of the device within the sheath 705 and then withdrawing the sheath 705 proximally. As the device 900 exits the distal end of sheath 705, it assumes the pre-formed device shape.

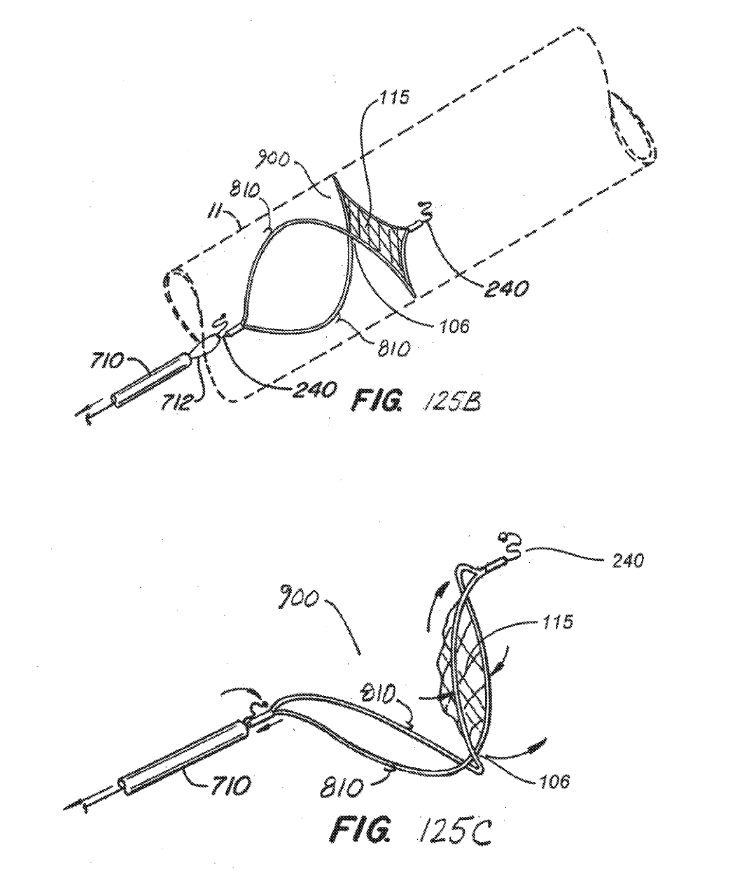

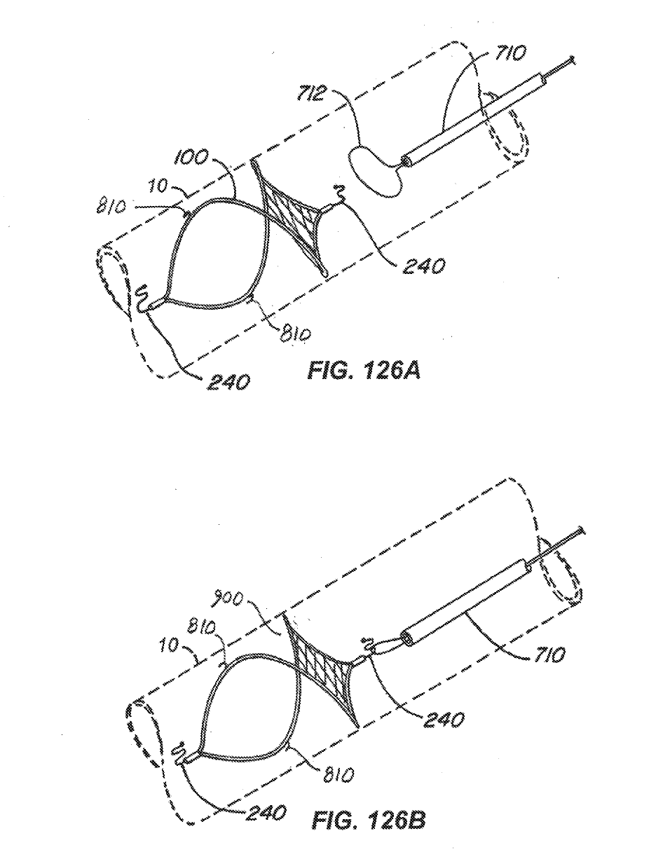

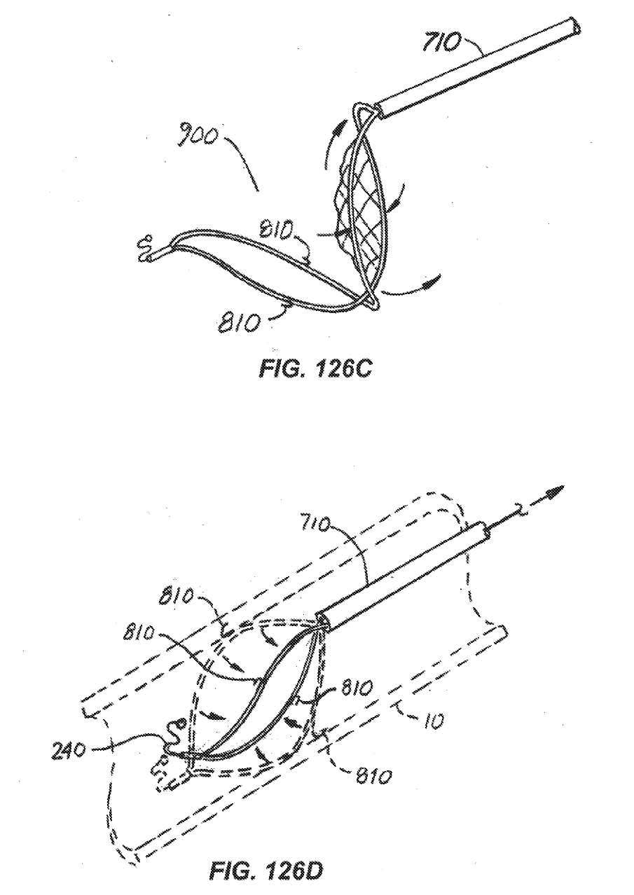

The symmetrical device shape (see e.g., devices in FIGS. 15, 16A, 96, 97, 90C, 99, and 88), facilitates the deployment and retrieval of the device 900 from multiple access points in the vasculature. As with other non-fixation filter devices described herein, a device 900 may be positioned as shown in the vasculature within the inferior vena cava 11 immediately below the renal veins 13 (see FIG. 70). A femoral access path (FIG. 126A) and a jugular access path (FIG. 125A) are illustrated. The femoral access path and a jugular access path may each be used for device deployment, repositioning and retrieval. Alternatively, the vena cava could be accessed via brachial or antecubital access for device deployment, repositioning and retrieval. The placement and orientation of the fixation elements or tissue engagement structures may be modified as needed to facilitate the desired placement and retrieval technique.

Retrieval of the devices is most preferably accomplished by endoluminal capture using one of the retrieval features described herein. (i.e., FIGS. 27A-E) The retrieval features described herein have been designed to work well using a commercially available snares two of which are illustrated in FIG. 71A and FIG. 71B. The single loop gooseneck snare 712 is illustrated in FIG. 71 inside of a recovery sheath 710. The multiple loop Ensnare 714 is illustrated in FIG. 71B inside of a recovery sheath 710. These conventional snares are controlled by a physician using a flexible, integral wire.

The sequence of device recapture and removal from a body lumen is illustrated and described above with reference to FIGS. 72A-C. A similar recovery sequence is used for embodiments of filter device 900 as illustrated in FIGS. 125A-125C. In this discussion, the device 900 is positioned in the vena cava. FIGS. 125A, 125B, and 125C illustrate an exemplary jugular recovery. The device 900 is illustrated within the vessel so that flow within the vessel initially passes through the material capture structure and then through the open support frame. FIGS. 126A-C illustrate an exemplary femoral recovery. The device 900 is illustrated within the lumen so that flow within the lumen initially passes through the material capture structure and then through the open support loop. A collapsed snare is advanced via a delivery sheath to the proximity of the retrieval feature 240. Once in place, the snare 712 is exposed and assumes a pre-defined expanded loop shape (FIGS. 125A and 126A). The loop shape is placed over the retrieval feature 240 as illustrated in FIGS. 125B and 126B. Advantageously, retrieval features of the present invention are positioned relative to and in contact with the luminal wall so that the feature may be more easily captured by a retrieval device such as a snare.

The snared device 900 can then be either pulled into the sheath 710, or alternatively and more preferably, the recovery sheath 710 is advanced over the device 900 while maintaining positive control of the snare 712 as the sheath 710 advances over the device 900. Advancing the recovery sheath 710 over the device 900 facilitates atraumatic removal of the device 900 from any tissue that has grown in or around the device 900. Additionally, the retrieval action, which tends to collapse the device radially inward (FIGS. 125C and 126C), also facilitates removal from any tissue layer formed on the device while also withdrawing the fixation elements from the lumen wall. Moreover, recovering the filtering device by pulling on a portion of the filter structure (i.e., a retrieval feature) removes the opposing spiral elements and the fixation elements or tissue engagement structure attached to them from the lumen wall. As the device 900 is drawn into the sheath 710, the pre-formed shape of the device 900 also urges the support members away from the lumen wall which also assists in retracting or disengaging fixation elements from the lumen wall (FIG. 126D).

Having discussed the various techniques and alternatives for positioning, deploying and retrieving a filter, a method of positioning a filter within a lumen will now be described. FIGS. 123A and 123B illustrate an embodiment of a step of advancing a sheath containing a filter through a lumen. FIG. 124A illustrates an embodiment of a step of deploying a portion of the filter from the sheath into the lumen to engage the lumen wall with a fixation device while maintaining substantially all of a material capture structure of the filter within the sheath. As shown in FIG. 124A, the retrieval feature 240 and at least one fixation element 810 have exited the sheath 705. The remainder of the filter including the material capture structure is still inside the sheath 705. Next, as shown in FIGS. 124B and 124C, is an embodiment of a step of deploying a support frame from the sheath to a position along and engaged with the lumen. The support frame is also used to engage fixation elements with the lumen walls. The shape and design of the support frame itself generates radial forces that also assist in securing the filter into position and maintaining the position of the filter within the lumen. FIG. 124C illustrates the support frame deployed from the sheath 705 and opened along the lumen 10. Two fixation elements 810 are shown engaged the lumen wall. The crossover 106 is also deployed. A portion of the material capture structure 115 adjacent the crossover 106 is also shown exiting the sheath.

Next is the step of deploying the material capture structure of the filter from the sheath into a position across the lumen. FIG. 124D illustrates the material capture structure exiting the sheath. A retrieval feature 240 is still inside of the sheath (shown in phantom).

FIG. 124E illustrates the fully deployed filter 900. The second retrieval feature 240 is in position against the lumen wall and the material capture structure is deployed across the lumen. FIG. 124E also illustrates an embodiment of the step deploying a filter retrieval feature 240 from the sheath 710 after the step of deploying another portion of the filter step.

In one embodiment, the filter illustrated in FIG. 124E could be modified to include a fixation element 810 on or near both retrieval features 240. In such an embodiment, as the last portion of the filter and the second retrieval feature exists the sheath 710 (the movement from FIG. 124D to 124E), another fixation element 810 at or near the second retrieval feature engages the lumen wall.

In another aspect, the method of positioning a filter may include the step of deploying a crossover structure of the filter into the lumen before or after the deploying the material capture structure of the filter step. One aspect of this step is illustrated in FIGS. 124B and 124C. These two views illustrate the partially deployed filter 900 having one retrieval feature and three engagement elements 810 out of the sheath 710 and into contact with the lumen. Additionally in this illustration, the crossover 106 has exited the sheath 710. In this stage of deployment, the engagement feature 240 is against one lumen wall, the crossover 106 is against another wall generally opposite to the retrieval feature 240. The deployed open support frame extends along the lumen between the crossover 106 and the engagement feature 240.

The collapsible nature of the filters of the present invention allows for filter recovery from the same direction that the filter was deployed as well as recovery from the opposite direction the filter was deployed. Embodiments of the filters of the present invention also reliably position retrieval features against the lumen wall to present in a way that is easy to snare. A filter may be deployed into the inferior vena cava using a femoral access route. Then that same filter may be recovered using an access route from the jugular or the superior vena cava as shown in FIG. 125A. Similarly, a filter placed into the vena cava using a jugular deployment route may be removed using a femoral approach as shown in FIG. 126A. In one specific example, the recovery is accomplished by maneuvering a snare towards the filter in the same direction used during the advancing step described above. Next, there is the step of engaging the snare with a filter retrieval feature positioned against a wall of the lumen. In an alternative technique, there is the step of maneuvering a snare towards the filter in the opposite direction used during the advancing step. Next, there is the step of engaging the snare with a filter retrieval feature positioned against a wall of the lumen.

The techniques for filter placement and recovery may be modified in other ways as well. For example, a method of positioning a filter as described above may be adjusted to include the step of deploying a filter retrieval feature from the sheath before the deploying a portion of the filter step. In another alternative, the step of placing the filter retrieval feature against the lumen wall may be performed before or after the positioning of a crossover within the lumen. Additionally or alternatively, the step of deploying a filter retrieval feature may also include placing the filter retrieval feature against the lumen wall.

Additionally, repositioning the filter 900 from one lumen position to another is accomplished in a similar fashion as described above with regard to FIGS. 74A-74D. Many embodiments of the device 900 have at least one atraumatic end such as illustrated in the non-limiting examples of FIGS. 90C, 91, 99, 96, 97, 94, 89C, 89A, and 88. In this context an atraumatic end is one that does not have any fixation or tissue engagement features. Because of the atraumatic design of these filter device embodiments, repositioning of the filter device 900 may be accomplished by fully recapturing (see FIG. 74C) or only partially recapturing (see FIG. 74B) the device 900 into a recovery sheath 710. By maintaining the portion of the device 900 having fixation elements contained within the sheath 710, the atraumatic end may be moved into the desired position and confirmed in position before deploying the remainder of the device and engaging the fixation elements. The atraumatic design of the device 900 allows the device to partially deploy such that only the atraumatic end is in the lumen. The partially deployed device may then be pulled along the lumen wall into the desired position. Once in position, the remainder of the device is then released from the sheath thereby allowing the fixation elements to engage with the lumen walls as they are freed from the sheath. The delivery sheath and recovery sheath are provided with the same reference numbers since filter devices of the present invention may be deployed into and recovered from the vasculature using sheaths that are about the same size. As such, devices of the present invention may be deployed into the vasculature from a delivery sheath having a first diameter. Then, the device may be retrieved from the vasculature using a recovery sheath having a second diameter no more than 2 Fr larger than the first diameter (1 Fr=0.013″=⅓ mm). Alternatively, the second diameter may be no more than 1 Fr larger than the first diameter or, alternatively, the first diameter is about the same as the second diameter.

While many of the features and alternative designs of fixation elements and tissue engagement structures have been shown and described with regard to FIGS. 88-125, it is to be appreciated that the invention is not so limited. The features and alternative embodiments described in FIGS. 83A-87 may also be applied to the various filters with fixation elements and tissue engagement structures. Additionally, the filters and embodiments described with regard to FIGS. 2A, 2B, 2C, 6C, 7D, 7G, 9A-10B, 11-19, 64A-67, 69A-87 may also be adapted to include any of the fixation elements or tissue engagement structures described or illustrated in FIGS. 88-126D.

Some filter embodiments may have material capture structures comprising intersecting filaments as described above. While some of the earlier described embodiments describe knotting of intersecting filaments, other embodiments comprise filaments joined in other ways. In some embodiments intersecting filaments are joined using beads, crimps, bonding, or adhesives. Such joining methods can advantageously decrease time and cost associated with knotting the filaments.

In some embodiments, the joining mechanism can be used for fluoroscopic visualization of the filter. Some filter embodiments may have material capture structures which include radiopaque materials, visible under fluoroscopy, to aid in determining the position of the material capture structures during and after deployment. Fluoroscopy refers to an imaging technique that uses X-rays to obtain real-time moving images of internal anatomy through the use of a fluoroscope. A radiopaque contrast agent can be injected into or near the site to be imaged to increase the visibility under x-ray imaging. The ability to visualize the material capture structure under fluoroscopy may help in ensuring the proper positioning of the device during deployment. During retrieval, the ability to visualize the material capture structure under fluoroscopy may help in assessing clot location and volume and ensuring proper capturing of the device. For example, the ability to visualize the material capture structure may help during retrieval by reducing the risk of entanglement between the retrieval device, which can be a snare, with the material capture structure. The ability to visualize the material capture structure may also help during adjunctive procedures performed in the area as the location of the material capture structure can impact various aspects of such procedures, such as selecting the route for accessing the treatment site. Various alternative embodiments of radiopaque markers are described below and can be adapted into a variety of combinations and configurations. The radiopaque materials used may include tungsten, tantalum, gold, barium, platinum, palladium, and bismuth used individually or in combination. Other materials are also possible. For example, platinum may be used in some embodiments, either individually or in combination with another radiopaque material.

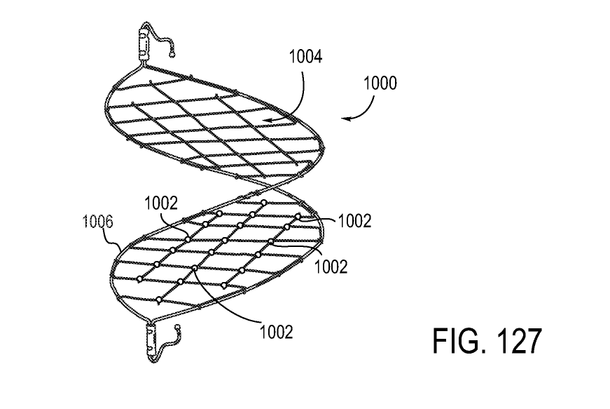

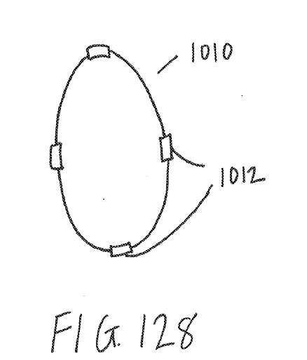

FIG. 127 illustrates an embodiment of a filter device 1000 with a material capture structure in each of two support frames. Other embodiments may only have a single material capture structure. The material capture structures include a plurality of filaments 1004. As described above, intersecting filaments of a material capture structure may be joined. The filaments 1004 in one of the material capture structures illustrated in FIG. 127 are joined by beads 1002. The beads 1002 are shown as approximately the same size and shape in FIG. 127; however, in some embodiments, the beads 1002 at different filament intersections may be differently sized and/or shaped. The beads 1002 may all comprise the same material or combination of materials. In some embodiments, the beads 1002 comprise different materials or combinations of materials. In some embodiments, some or all of the beads 1002 can be radiopaque. FIG. 127 illustrates the beads 1002 positioned only at the intersection of filaments 1004. In some embodiments, however, marker beads 1002 may be positioned on the filaments 1004 in between the intersections. The beads 1002 are positioned at each intersection of filaments in one of the material capture structures of device 1000. In some embodiments, beads may be positioned at only some of the intersections of filaments.

Radiopaque marker beads included in the material capture structure may be selected based on the desired fluorogenic properties of the filter device. For example, the materials, sizes, and shapes used for the radiopaque portions of the device may impact the fluorogenic visibility of the device, which may influence the number of marker beads used. In some embodiments, the size of the marker beads is limited so that the marker beads do not substantially impede the flow of blood across the filter. For example, the marker beads collectively may occupy less than about 5, 4, 3, 2, or 1 percent of the surface area defined by the material capture structure.

Each individual bead 1002 may comprise a single material or a combination of materials. The beads 1002 may have one of a variety of shapes, including a round shape (for example, generally spherical, generally ovular), a rectangular shape (for example, generally square shaped), and triangular shaped. In some embodiments, the marker 920 may be cylindrically shaped or ring shaped, appearing as a band (e.g., a marker band for radiopaque materials).

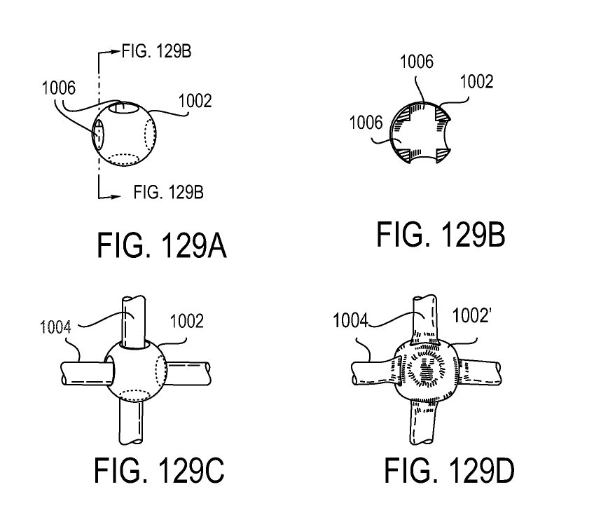

In some embodiments, the bead 1002 includes lumens 1006 through which the two intersecting filaments are configured to extend, as shown in FIG. 129A. The lumens may intersect generally perpendicularly, forming a ‘t’ shape (FIG. 129B). In some embodiments, the lumens intersect at an angle other than 90°. For example, the lumens may intersect at an angle between about 0° and about 90°. In some embodiments, the bead 1002 may join the filaments 1004 without crossing the filaments 1004. In such embodiments, the lumens in the bead 1002 would not intersect. In some embodiments, the beads may be used to join more than two filaments, for example in the filtering structure configurations shown in FIGS. 59D-59G.

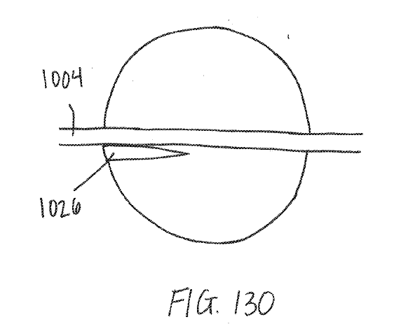

As described above, in some embodiments, beads (e.g., radiopaque marker beads) can be positioned away from the intersection of filaments. For example, marker beads can be positioned around a filter cell. Filter cells can be formed in a variety of ways, including by overlapping or intersecting filaments. The filter cell 1010 of FIG. 130 includes radiopaque marker beads 1012 positioned around the filter cell. The spacing of the markers beads 1012 is shown as being approximately equal in FIG. 130. In some embodiments, the spacing of the marker beads may not be equal. For example a density of marker beads in a first section of the filter cell may be greater than a density of marker beads in a second section of the filter cell. The filter cell 1010 of FIG. 130 includes four marker beads 1012. However, other numbers of marker beads 1012 may also be used. A number of marker beads included in the filter cell may be selected based on the desired fluorogenic properties of the filter device. For example, the materials, sizes, and shapes used for the radiopaque portions of the device may impact the fluorogenic visibility of the device, which may influence the number of marker beads used. The marker beads 1012 illustrated in FIG. 128 are all approximately the same size and shape. In some embodiments, a filter cell includes marker beads of varying sizes and shapes. In some embodiments, the marker beads 1012 comprise a same material. In other embodiments, the marker beads 1012 comprise different materials. In some embodiments, the beads 1012 are not radiopaque.

Each individual bead 1012 may comprise a single material or a combination of materials. The beads 1012 may have one of a variety of shapes, including a round shape (for example, generally spherical, generally ovular), a rectangular shape (for example, generally square shaped), and a triangular shape. In some embodiments, the bead 1012 may be cylindrically shaped or ring shaped, appearing as a band, like the marker beads in FIG. 128. Radiopaque marker bead and marker bands can be referred to generally as discrete radiopaque markers. In some embodiments, the beads 1012 are formed by extruding a material onto the filter cell. In examples of radiopaque beads 1012, the material would comprise a radiopaque material. In some embodiments, the bead 1012 includes a lumen through which a filament of the filter cell is configured to extend, similar to lumen 1006 of marker bead 1002. In some embodiments, the bead comprises a separate structure such as a sheath, crimp, clip, etc. that can be joined to the filter cell.

In some embodiments, the material comprising the beads 1002, 1012 is radiopaque. In other embodiments, the beads 1002, 1012 are coated with a radiopaque material. The coating may coat an entire surface of the marker bead or just a portion of the surface, for example a band around the bead. In beads including lumens, the lumen, or at least a portion of the lumen, may be coated with a radiopaque material. For example, in some embodiments, the lumens are lined with a polymer that can be doped with a radiopaque material. The dopant concentration may be between about 0.01% and about 99%. In some embodiments, the dopant concentration is between about 20% and about 70%. In some embodiments, the beads 1002, 1012 include a radiopaque interior portion, such as a radiopaque core. It will be appreciated that the term “radiopaque” includes materials that are naturally radiopaque and non-radiopaque materials doped with a radiopaque material.

In some embodiments, the beads 1002, 1012 are fixed to the filament or intersection of filaments by crimping or swaging, or thermal fusing or gluing the bead onto the filament or the like. The beads 1002, 1012 may be joined to the filaments using an adhesive, in some embodiments. In some embodiments, the lumens of the beads 1002, 1012 may be lined with a material that can be treated to join the bead to the filament or filaments. FIG. 129A depicts a bead 1002 including two lumens 1006. A cross-sectional view of the bead 1002 taken along line FIG. 129B-FIG. 129B is provided in FIG. 129B. At least a portion of the surface of the lumens 1006 has been coated with a material, such as PTFE or FEP. FIG. 129C illustrates the bead 1002 with the filaments 1004 inserted through the lumens 1006. FIG. 129D depicts the bead 1002 and filaments after treatment, such as heat melding, resulting in the at least partial to complete fusing of the bead 1002′ and the filaments 1002. The bead 1002′ is shown as slightly deformed following treatment. In some embodiments, the bead 1002′ will not be deformed following treatment. It is to be appreciated that the treatable material may be formed, for example, from: ePTFE, PTFE, PET, PVDF, PFA, FEP, and other suitable polymers. In some embodiments, the beads can be fixed to the filament by wedging an object such as a shim 1026 (FIG. 130) into the bead lumen, pressing the filament against an inner surface of the bead lumen to at least partially or completely reduce mobility of the filament within the lumen. FIG. 130 illustrates one shim 1026 being used to fix the bead in place. More than one shim may be used, in some embodiments. The shim 126 shown in FIG. 130 is wedge-shaped. Other configurations are also possible. The bead lumen may have a widened portion to allow for placement of the object 1026. In some embodiments, the bead may additionally or alternatively be secured to the material capture structure using a heat shrink polymer wrapping or covering that can be disposed over the bead and then heat treated.

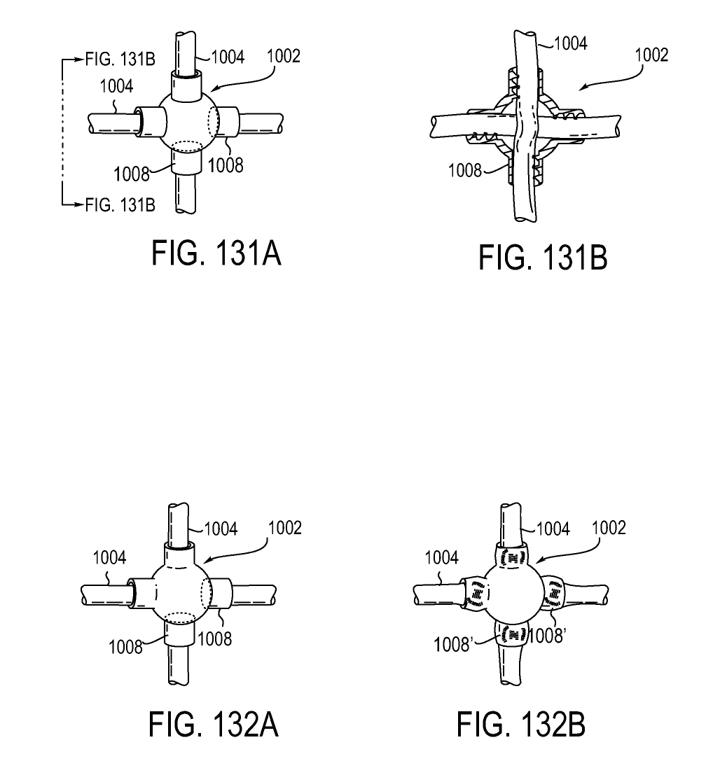

In some embodiments, the beads 1002 may include protrusions 1008 which can increase the bead surface area contacting the filament. Increased surface area contact may advantageously allow for a stronger connection between the filament and the bead. FIG. 131A depicts a bead 1002 including protrusions 1008. The protrusions 1008 are shown as cylindrical; however, other shapes are also possible. For example, the protrusions may have a rectangular or triangular cross section. The protrusions 1008 are also shown as having a uniform shape and size. In some embodiments, a bead may include protrusions of different sizes and/or shapes. The protrusions may be formed integrally with the beads. Alternatively, the protrusions may be separate structures joined to the beads. For example, the protrusions may be cylindrical inserts that can be inserted into and joined with the bead. For another example, the protrusions may not be inserted into the bead and may instead be joined to an external surface of the bead surrounding a bead lumen opening. Separate formation of the protrusion and the bead can allow for the selection of materials to serve a particular purpose, rather than more than one purpose. For example, at least a portion of the bead can comprise a radiopaque material and at least a portion of the protrusion can comprise a material suitable for joining to the filaments.

FIG. 131B depicts a cross sectional view of the bead 1002 of FIG. 131A taken along the line 131B-131B. The protrusions 1008 include ridges or teeth 1009 configured to contact the inserted filament 1004. The bead 1002 can be crimped or swaged onto the filaments 1004, as shown in FIG. 131B, at least partially or completely joining the bead and the intersection of the filaments 1004.

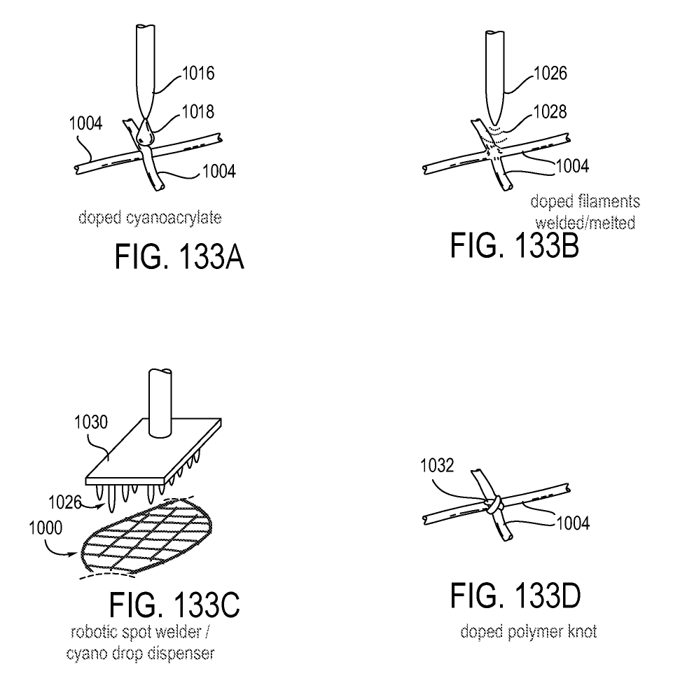

FIGS. 132A and B depict an alternative embodiment of a bead 1002 including protrusions 1008. FIG. 132A depicts the filaments 1004 inserted within the lumens of the bead 1002 including protrusions 1008. FIG. 132B illustrates the bead 1002 and filaments after suitable treatment, for example heat melding, to bond the bead 1002 to the filaments 1004, at least partially or completely joining the bead and the intersection of the filaments 1004. The treated protrusions 1008′ of FIG. 133B have deformed slightly as a result of the treatment. In some embodiments, the protrusions 1008 or bead 1002 will not deform as a result of the treatment. In some embodiments, the entire bead 1002 may comprise a material that may be treated. Alternatively, only portions of the marker bead 1002 may include a treatable material. As described above, in some embodiments, only the protrusions comprise a material that may be treated, as depicted in FIG. 132B. For example, an inner surface of the protrusions may include a polymer that can be bondable. It is to be appreciated that the bondable material may be formed, for example, from: ePTFE, PTFE, PET, PVDF, PFA, FEP, and other suitable polymers.

In some embodiments, the beads have a diameter or thickness of about 0.05 mm to about 8.0 mm. In some embodiments, the beads have a diameter or thickness of about 0.1 mm to about 7.0 mm. In some embodiments, the beads have a diameter or thickness of about 0.015 mm to about 6.0 mm. In some embodiments, the beads have a diameter or thickness of about 0.2 mm to about 5.0 mm. Other sizes are also possible. For example, the beads can have a diameter or thickness of less than about 0.01 mm. The size of the beads may be selected to provide a desired visibility under fluoroscopy.

In some embodiments, the filaments 1004 themselves are doped with a radiopaque material. In such embodiments, all of the filaments 1004 of a material capture structure may be doped. Alternatively, only a subset of the filaments may be doped. For example, a subset of filaments forming a pattern (for example, alternating filaments) across the material capture structure can be doped. For another example, filaments or portions of filaments in one or more areas of the material capture structure can be doped. A filament 1004 may be doped along its entire length or along only a portion of its length. A filament 1004 may be doped with radiopaque material during manufacture of the filament 1004. In some embodiments, a radiopaque material is applied to the already formed filament 1004. FIGS. 133A-D illustrate various embodiments of doped filaments 1004. FIG. 133A depicts an example of two filaments 1004 being joined by doped cyanoacrylate 1118. Other doped adhesives or bondable materials can also be used. FIG. 133A depicts an applicator 1016 applying the doped cyanoacrylate 1118. FIG. 133B depicts an embodiment in which the filaments 1004 themselves are doped and melded together through treatment 1028 (for example, heat treatment, application of adhesive) to the intersection of the filaments 1004 via applicator 1026. FIG. 133C illustrates a device 1030 comprising a plurality of applicators 1026 that can be used to either spot treat (for example heat meld, weld, apply adhesive, etc.) multiple intersections of filaments in a filter device 1000. FIG. 133D depicts an intersection of two filaments 1004, wherein the filaments are joined by a doped material (for example, a polymer) 1032. The doped material 1032 is knotted around the filaments 1004, joining the filaments 1004. In some embodiments, the filaments may be coated with a doped material, such as a polymer doped with a radiopaque material.

The filaments 1004 may have a dopant concentration from greater than 0% to about 99%. In some embodiments, the dopant concentration is between about 20% and about 70%. For example, a filament comprising PTFE may be doped with tungsten, at a concentration of between about 20% and about 70%. Other materials are also possible. For example, a filament may comprise FEP and be doped with platinum, at a concentration of between about 30% and about 60%.

In some embodiments, the filaments comprise a hollow outer filament and a radiopaque filament core. For example, the outer filament can comprise PTFE and the core can comprise platinum. In some embodiments, the core is continuous throughout the filaments. In some embodiments, the core comprises discrete sections positioned along the length of the filament.

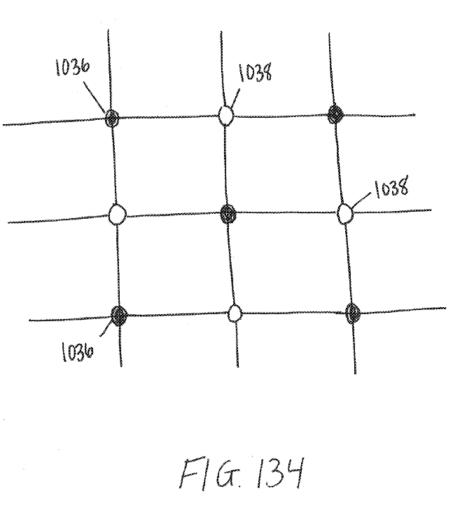

While the previous description related to structures that can include fluorogenic markers, in some embodiments, the filtering device may also include echogenic markers. The filtering device may include only fluorogenic markers or only echogenic markers. In some embodiments, the device is visible using co-registration and includes a combination of fluorogenic and echogenic markers. FIG. 134 depicts an embodiment of a material capture structure including fluorogenic marker beads 1036 and echogenic marker beads 1038. The echogenic marker beads 1038 can incorporate an echogenic material such as air (or other gas) bubbles or glass (or polymeric, metallic or other) beads. Other echogenic materials are also possible. For example, glass fibers may be incorporated into an echogenic marker bead. In embodiments of filter devices including fluorogenic and echogenic markers, the device may include an equal number of each type of marker. In some embodiments, the device includes a greater number of fluorogenic markers than echogenic markers. In some embodiments, the device includes a greater number of echogenic markers than fluorogenic markers. Echogenic marker beads can be of various sizes, shapes, and configurations as described above with respect to the fluorogenic marker beads. The echogenic marker beads can be joined to filaments or intersections of filament in a variety of manners, including those described above with respect to the fluorogenic markers.

While many of the features and alternative designs of joining mechanisms (e.g., beads) which can include fluorogenic and echogenic markers have been shown and described with regard to FIGS. 127-134, it is to be appreciated that the invention is not so limited. The features and alternative embodiments described in FIGS. 83A-87 may also be applied to the various filters with beads that may include fluorogenic and/or echogenic markers. Additionally, the filters and embodiments described with regard to FIGS. 2A, 2B, 2C, 6C, 7D, 7G, 9A-10B, 11-19, 64A-67, 69A-126D may also be adapted to include any of the fixation elements or tissue engagement structures described or illustrated in FIGS. 127-134.

In some embodiments, the material capture structure can be formed by laser cutting a sheet of material to form a mesh or other material capture structure form. Laser cutting a material capture structure can advantageously be simpler to manufacture than cutting and joining individual filaments. A pre-assembled web can lower manufacturing time. Multiple pre-assembled webs can be built and set aside for further use. Additionally, not having any joining mechanism (e.g., knot, bead, etc.) between individual filaments can improve flow characteristics of the filer and reduce potential for thrombosis.

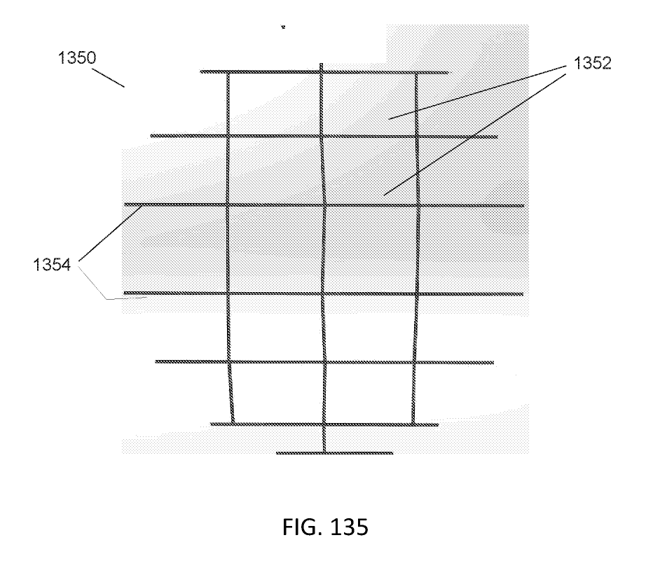

A laser cut material capture structure can be formed from materials such as ePTFE, PTFE, PET, PVDF, PFA, FEP, other suitable medical grade polymers, other biocompatible polymers and the like. FIG. 135 illustrates an embodiment of a laser cut material capture structure prior to attaching it to the filter frame. The mesh can be formed from, for example, raw PTFE, then expanded to form ePTFE. The mesh 1350 of FIG. 135 comprises generally rectangular shaped cells 1352, but other shapes are also possible. For example, the cells may have any, one or more, or hybrid combinations of shapes such as, for example, circular (FIG. 60A), polygonal (FIG. 60B), oval (FIG. 60C), triangular (FIG. 60D), trapezoidal or truncated conical (FIG. 60E). Any of the designs described herein, for example, shown in FIGS. 59A-63B, are also possible.

As discussed elsewhere herein, components of the filter can be adapted and configured to contain a drug or combination of drugs or pharmacological agents that are released over time or after some initial time delay. For example, the material forming the material capture structure (e.g., raw PTFE) can itself include a doped medication (e.g., heparin). In other embodiments, the medication can be dipped into and absorbed onto the outside of the formed material capture structure (e.g., formed of ePTFE). In some embodiments, the material capture structure comprises two sheets of joined laser cut material, and a drug, such as heparin, can be injected in between the two joined sheets.

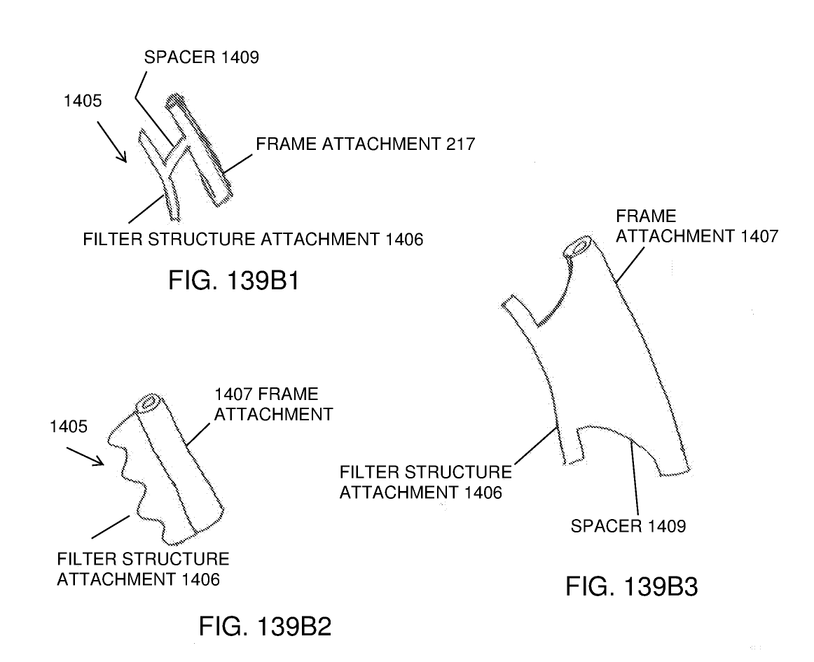

As shown in FIG. 135, in some embodiments, the ends 1354 of the mesh can be left longer in length to accommodate the slack needed for tying the web. In some embodiments, the material capture structure may be configured or fabricated so as have an edge or perimeter for being attached directly to the support frame. In alternative configurations, a new material capture structure or a conventional material capture structure is attached to a securing element that is in turn attached to a support frame either with or without an intervening spacing element. For example, the ends of the material capture structure can comprise flat tab portions, integrally formed or attached thereto and configured to be folded over the frame. The material capture structure can include any of the edges configured for direct attachment, securing elements, or spacers shown in FIGS. 139A-139E.

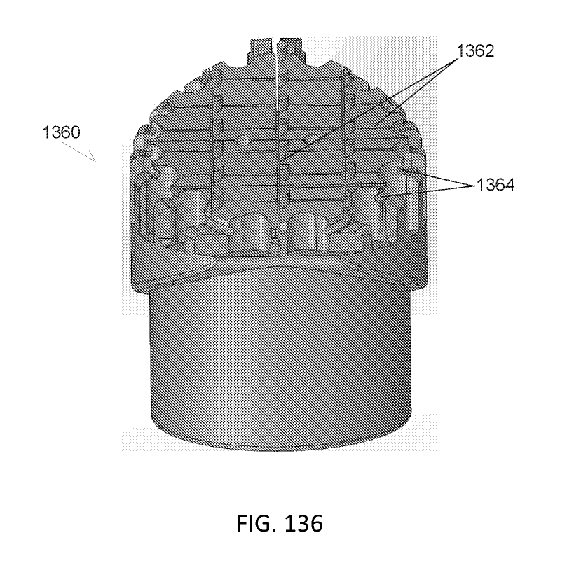

FIG. 136 illustrates a material capture structure attaching fixture 1360. The mesh 1350 of FIG. 135 can be placed into the slots 1362 of the fixture 1360. In some embodiments, the filter frame can be placed in the perimeter slot 1364. The material capture structure can be attached by tying, welding, gluing, bonding, joining, crimping, etc. to the filter frame support members. In some embodiments, the material capture structure comprises edges configured for direct attachment, securing elements, or spacers, as described above, used for attaching the material capture structure to the filter frame. Placing the mesh 1350 within the slots 1362 can prevent the mesh from moving during pre-assembly, which can create a more consistent attachment to the filter frame (e.g., between knots). More consistent attachment can help increase tensile/web strength of the material capture structure.

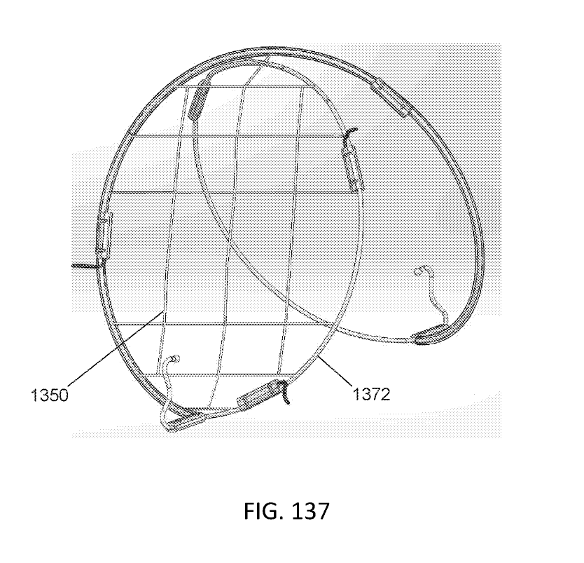

FIG. 137 illustrates an example of the material capture structure of FIG. 135 attached to a filter frame 1372.

Some of the above described embodiments relate to alternatives to material capture structures formed by knotting filaments, such as joining filaments using beads or laser printing of a material capture structure. Some of the following embodiments relate to material capture structures or structure components that can be formed using 3D printing.

As used herein, the general term “3D printing” or “printed” refers to the use of one or more of the generally accepted 3D printing technologies otherwise known as additive layer manufacturing (“ALM”) or additive manufacturing. By way of general categorization, 3D printing or ALM is related to the use of one or more of six core processes. These additive manufacturing or 3D printing processes are: photocuring (represented by SLA, DLP and MJM), sintering (SLS and DMLS), melting (EBM and DMP), lamination (LOM), extrusion (FDM and PJP) and, finally, jetting (MJM,SCP and DMP).

In one aspect of an embodiment of the present invention, a suitably formatted Computer Aided Design (CAD) file is prepared including three dimensional design information and details for one or more aspects of the support frame, material capture structure, structure attachment element or medical textile pattern, weave, strand size, strand shape, strand overlap, strand intersection geometry, between strand spacing in any dimension of the weave design or other characteristic of the medical textile pattern or weave to ensure fabrication via any suitable additive fabrication or 3D printer system. The CAD file may be generated using standard computer design software 3D modelling program or by the use of a prototype and appropriate 3D scanner or any combination thereof. In one aspect, the feed or source material used in the ALM process is a biocompatible material, a bioresorbable material or a biodegradble material. In still another aspect, the feed or source material used in the ALM process is compatible with one or more sterilization processes used in the medical device industry.

The process described above may be useful in a wide variety of designs to simplify a medical device assembly process. In aspect, a material capture structure is attached to a filter support frame using one or more printed connector or attachment structures. The printed attachment or connector structures have at least one attachment point adapted and configured for attachment to the material capture structure. In addition, the printed attachment or connector structures have at least one attachment point adapted and configured for attachment to a portion of the support frame. In this way, a variety of attachment points—optimized for the filter components to be joined or assembled—may be more readily designed and fabricated with ease of attachment and assembly as a portion of the filter printed attachment or connector structures have at least one attachment point adapted and configured for attachment to the material capture structure.

In still other aspects, a medical textile is designed using appropriate 3D design software to designate the individual strands of a weave pattern suited to use in a medical device. In one aspect, the medical textile has a weave pattern specified for 3D printing for use as a vascular graft. In one aspect, the medical textile has a weave pattern specified for 3D printing for use as an occlusive device or as a filter. In one specific embodiment, the filter is selected to have a spacing size or filtering dimension suited for distal protection procedures. In one specific embodiment, the filter is selected to have a spacing size or filtering dimension suited for filtering uses for embolic protection.

In one embodiment, there is a 3D model of the medical textile or filter element constructed using an appropriately configured virtual peg board to map the pattern of the strands to form the medical textile or filter or knots used in the formation thereof as described herein. Once the path of each of the various elements of the filter or the attachment point or other design aspect is input, then the strand elements are mapped using control points to produce the various different planes of strand movement used in weave and fabric design. The medical textiles described herein may be formed to include textile or filter designs with two planes, three planes, four planes or more depending upon the complexity of the weave pattern in the medical textile or filter design. In addition or alternatively, the overall filter shape may be determined in the design and the individual strand parameters adjusted, adapted or altered accordingly. The medical textiles and filter designs include, in various alternative aspects, a number of various patterns, curves, geometry, shape, strand intersection pattern, strand intersection geometry, strand intersection style, filter cell size, filter pore size, filter cell shape, filter cell orientation based upon the physiological and anatomical requirements of the ultimate medical textile or filter.

Advantageously, since each strand is individually fabricated using the techniques described herein, each one of the strands used in the fabrication of the medical textile or filters may be set to unique characteristics, designs, shape, geometry including the use of varying geometry along the length of a strand or as needed for the portion of a strand in its particular location in a medical textile or filter design. In one aspect, a strand in a 3D printed medical textile or filter has a cross section shape and overall dimension having one or more controlled (i.e., designed) variations in the cross sectional shape, size or contours of a strand at a particular point in the medical textile or filter design. As a result, one strand may have a constant cross section shape and overall size or a variable cross section shape and overall size. In one aspect, the cross section shape of one or more strands in a printed medical textile or filter is one or more of: circular, oval, elliptical, rectangular, trapezoidal, triangular, pentagonal, octagonal or other multiple sided shape.

In one aspect, the surface finish or shape of a strand is selected based upon the position of the strand in the medical textile or filter when the textile or filter is in use. In one specific example, the cross sectional shape and or surface finish of a strand has a first shape and finish for positioning of the strand or a portion thereof against a lumen or tissue surface and another or second variable shape and finish for positioning of the strand or a portion thereof for implant within the body but not against a lumen or tissue surface. In one specific example, there first surface shape and finish is selected for contact against a wall of a vessel such as a vein or an artery. The second surface shape and finish is selected based on flow dynamics in the vessel. By way of this example, the first shape and finish may be selected to induce or reduce or minimize tissue growth or degree of damage to the wall. The second shape and finish may be selected based on designs to reduce turbulence between the flow in the lumen and the second strand surface.

Advantageously, because the design and manufacturing process described herein permit each strand to be individually designed, surface and finish of each strand may be designated based upon the various different criteria for the “in use” environment of the medical textile or filter. In one aspect, a strand may be configured to have one or more interior conduits or lumens extending along at least a portion of the strand length. One or more conduits of one or more strands may be provided within the interior of the filter, device or graft. A suitable fluid connector is also provided in a portion of the device, filter or graft to provide a fluid path way into the strand based fluid conduits. One or more apertures are provided in the stand lumens to permit fluid introduced into the fluid connection to be delivered into the in use location of the filter, device or graft. In still another alternative, a component or element of a medical device, filter or graft designed and fabricated via an ALM process is adapted and configured to include one or more lumens or apertures as described above. In still other embodiment, there is provided a material capture structured formed as described herein wherein the apertures are normally closed slots but when a force is exerted on the strand or lumen bearing component, the normally closed slot or aperture will open. In one specific embodiment, the normally closed aperture is in communication with a lumen bearing strand or filter or other component wherein the lumen contains a lytic drug or other pharmacological agent. In one specific embodiment, the normally closed aperture is opened and the pharmacological agent is released when a clot or thrombus is trapped and a strain is induced on the web. As a result of interaction with a clot or debris, the normally closed apertures are opened.

In one aspect, the component or element has an overall cross section shape and size of about 0.01 mm, 0.02 mm, or between 0.01-0.1 mm, or between 0.1 and 1 mm, or 2-4 mm, or 4-6 mm or up to 10 mm or other size according to the design criteria of the device, filter or graft. In still other alternative aspects, the strands in a printed medical textile, device or filter have a circular cross section shape and have a diameter of about 0.01 mm, 0.02 mm, or between 0.01-0.1 mm, or between 0.1 and 1 mm. In one aspect, the strand or element has an outer diameter of about 0.01 mm. Various details of printed fabric are found at http://newtextiles.media.mit.edu/?p=3162, published May 9, 2012.

It will be appreciated that the manufacturing processes described with respect to 3-D printing can also be used in manufacturing of components formed in other ways, for example, by laser cutting.

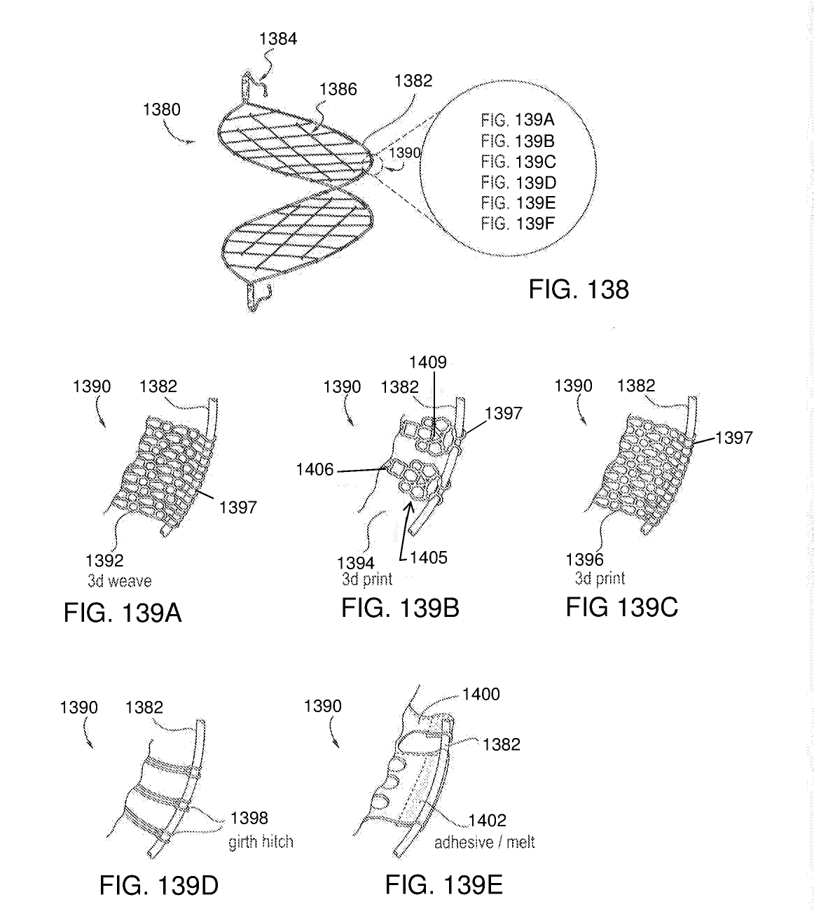

FIG. 138 is a perspective view of a dual loop filter support frame having a material capture structure within and secured to the frame. In this illustrative embodiment, the material capture structure may be formed from one or more of the techniques herein to produce a filter structure for direct application to the support frame. In one alternative aspect, there may also be one or more securing elements formed using one or more of the techniques described herein to simplify and enhance the securement of a material capture structure to a support frame. In one aspect, there is at least one of the material capture structure, the capture structure securing device or both formed by or using one or more techniques illustrated in FIGS. 139A through 139E. While illustrated as a double loop, the techniques and structures described herein could be applied to support frames or other shapes and styles depending upon the specific anatomical considerations of a specific filtering environment or the specific shape and geometry of a support frame. For example, there may be a support frame or other components as provided in U.S. patent application Ser. No. 13/553,335, filed Jul. 19, 2012, titled “ENDOLUMINAL FILTER WITH FIXATION,” Publication No. US-2013-0012981-A1 and International Pat. App. No. PCT/US2008/088606, filed Dec. 31, 2008, titled “ENDOLUMINAL FILTER WITH FIXATION,” Pub. No. WO 2009/088905, each of which is incorporated herein by reference in its entirety. While illustrated as having a material capture structure in both loops, there may be embodiments with a material capture structure formed as described herein is only one of several provided support frames in a particular filter structure (i.e., multiple loop support frame having only one material capture structure).

As will become clear in the description that follows, there are embodiments of filtering devices according to the present invention with a material capture structure formed by a biocompatible additive manufacturing process. This new material capture structure may be configured or fabricated so as have an edge or perimeter for being attached directly to the support frame. In alternative configurations, a new material capture structure or a conventional material capture structure is attached to a securing element that is in turn attached to a support frame either with or without an intervening spacing element.

FIG. 139A illustrates an enlarged view of a material capture structure formed from a 3D printed weave with a portion of the perimeter shown attached to the support frame. The filter 1390 in this embodiment includes a material capture 1392 formed from an additive manufacturing process to produce a weave pattern. It is to be appreciated that the weave formed may take on the properties of a variety of different textiles in order to provide the desired filter cell size or filtering capacity for the resulting material capture structure or filtering device. In this embodiment the filter cells are illustrated with a repeated pattern of almond, walnut or macademia nut and circular shaped openings. The edge of the capture structure is adapted and configured for direct attachment to the perimeter of the corresponding frame and to the size and shape of the frame 1382.

As best seen in, for example, FIGS. 139A and 139C, an edge 1397 may be formed into a number of different configurations. In one embodiment, the edge is a plurality of strands that are formed into a hook shape. The hook shape may be sized and shaped to conform to the outer wall of the support frame 1382. The length of the strands in the edge may be selected to partially encircle, fully encircle or make one or multiple turns about the support frame 1382. In any of these embodiments, the strands of the edge 1397 may wind into loops, spirals or helical wraps about the support frame that are contacting, spaced apart or combinations thereof depending upon the desired configuration. In one aspect, the edge 1397 may also be shaped and configured for a friction fit over the support frame 1382. Alternatively, the edge 1397 may be shaped and configured to fit over and then subsequently treated to for the final secure fit with the support frame. The treatment of the edge 1397 may include one or more of a thermal, chemical or light based process to accomplish the later edge securing step.

FIGS. 139B and 139B1-139B3 illustrate various embodiments of a securing element 1405. In general, a securing element 1405 is attached to both a material capture structure and a support frame. The securing element 1405 may be integrally formed with a material capture structure or it may be a separate element that is attached to the material capture structure so as to simplify attachment of a material capture structure to a support frame. The general components of a securing element 1405 are best seen in FIG. 139B1. The securing element 1405 has a filter attachment portion 1406 that is sized, configured and adapted for ready attachment to a material capture structure directly or to a perimeter of the capture structure. The frame attachment 1407 serves a similar purpose for securement to the support frame 1382. In the illustrative embodiments of FIGS. 139B1, 139B2 and 139B3, the frame attachment elements 1407 are illustrated as short tubes for a generally circular cross section support frame. Other configurations of the frame attachment are possible and are varied according to the specific support frame to be used.

As shown in FIG. 139B1, there is also an optional spacer 1409 used to bridge the gap (if any) between the size and shape of a material capture structure and the support frame that will deliver the capture structure within the body. While a single centrally placed spacer 1409 is illustrated in this embodiment, there be more than one or multiple orientations of the spacer 1409. Moreover, plural spacers 1409 of several orientations, size or spacing may be disposed between a filter structure attachment 1406 and corresponding frame attachment 1407. As such, it is to be appreciated that FIGS. 139B1-139B3 illustrate alternative embodiments of material capture structure securing elements 1405 having variable spacer configurations such as a discrete spacer (FIG. 139B1); no spacer (FIG. 139B2) and web spacer (FIG. 139B3). It will be understood that such spacers can also be used with other filter embodiments described herein, for example those filters that are laser cut.

By way of example, the perimeter of the material capture structure is aligned as suited to the specific application of the filter being constructed. In one aspect, there is a difference in the final filtering device based on the type, size, spacing or appearance of gaps between the material capture structure and the support frame. In one exemplary embodiment, there is a support frame and material capture structure combination where any gap between the perimeter or boundary of a material capture structure and frame is as small as practical or considered eliminated as compared to the size of the related filter cell size. In another exemplary embodiment, there is a material capture structure and support frame combination where small gaps—both regular sized and spaced gaps or irregularly sized spaces and gaps are permissible in the design given the filtering environment. In still another aspect, there is provided a material capture structure having 100-200 micron sized cells or pores for use in distal protection. In still other embodiments, there is provided a material capture structure with cell sizes selected for filtering ranges of 5-8 mm.

FIG. 139B illustrates an enlarged view of a material capture structure having an 3D printed securing structure 1405. In this illustrated embodiment, the securing element 1405 is formed along a portion of the material capture structure perimeter and shown attached to the support frame 1382. The frame attachment 1405 may be formed similar to the edge 1397 described above or may take other aspects as in the frame attachments described herein. In this illustrative embodiment, the spacer 1409 has a number of openings formed into a filtering pattern. The use of a spacer 1409 having a filtering pattern may have advantageous use when there exists a gap between a material capture structure and the support frame where a single spacer (see FIG. 139B1) is undesired. In the configuration of FIG. 139B, the portion of any gap between material capture structure and the support frame may be filtered by the pattern provided in the spacer 1409. The spacer 1409 illustrated in FIG. 139B the gap between the material capture structure is also part of the filtered area using the pattern formed. In contrast, the spacer 1409 may be formed to occlude any gaps formed between the material capture structure and the support frame. FIG. 139B3 illustrates an embodiment of a securing element 1405 where the spacer 1409 is now a solid sheet that spans the distance between the filter attachment 1406 and the frame attachment 1407. In still another alternative embodiment, the spacer 1409 is removed. FIG. 139B2 illustrates a securing element embodiment where the filter structure attachment 1406 is coupled directly to the frame attachment 1407.

FIG. 139C illustrates an enlarged view of a material capture structure printed using a form of 3D printing with a portion of the perimeter shown attached to the support frame. In this illustrative embodiment, there is an edge 1397 formed on the material capture structure 1396 that is used to secure the material capture structure 1396 to the support frame 1382 as describe above. The material capture structure 1396 may be used to print a variety of patterns to produce filter cells with the material capture structure that are sized, shaped, configured, arranged or otherwise disturbed within and about the material capture structure 1396 depending upon the specific filtering requirements of the filtering structure.

FIG. 139D illustrates an enlarged view of a material capture structure having a number of separate attachments 1398 along the material capture structure perimeter. In this embodiment, each attachment 1398 is shown tied about and secured to the support frame 1382. In this exemplary embodiment, each attachment 1398 is used to form a girth hitch or cow hitch about the support frame 1382. In one embodiment, the lines used form one or more separate attachments are formed as part of the material capture structure as when a 3D printed material capture structure also forms the lines needed to secure to the support frame in the desired technique. Alternatively, the separate lines used to form attachments are attached to a material capture structure in a suitable manner to permit the lines to be used in the manner illustrated in FIG. 139D.

FIG. 139E illustrates an enlarged view of a filter 1390 having a material capture structure formed with a series of securing tabs 1400 along a portion of the perimeter. In this illustrative embodiment, two securing tabs 1400 are shown folded over the support frame and then attached back onto itself. In one aspect, there is an over lapping region 1402 formed by a fold of the tab about the support frame. The overlapping portions of the tab 1400 may be joined together in any suitable way such as with an adhesive or melted or fused together. In still other embodiments, coupling elements are formed in, attached to or included in a printed portion of the tab. In this aspect, the tab is folded over the support frame and then held in place by joining the coupling elements. The coupling elements may be any of a wide variety of male-female configured friction fit elements. In still other aspects, the coupling elements may include one or more features similar to a ratchet or lock to ensure that the coupling elements remain engaged.

In addition to the various embodiments of material capture structures described herein, the techniques for forming a material capture structure or attaching a material capture structure to a support frame may be applied to a wide variety of material capture structures. More particularly, the different variations of material capture structure 1390 may be suitably affixed to a support frame using a variety of techniques. In each of the embodiments that follow, a different material capture structure embodiment is illustrated as attached to one loop of a double loop structure. It is to be appreciated that the techniques described herein may be applied to different types of support frames or by using more or fewer of the attachment techniques described herein.

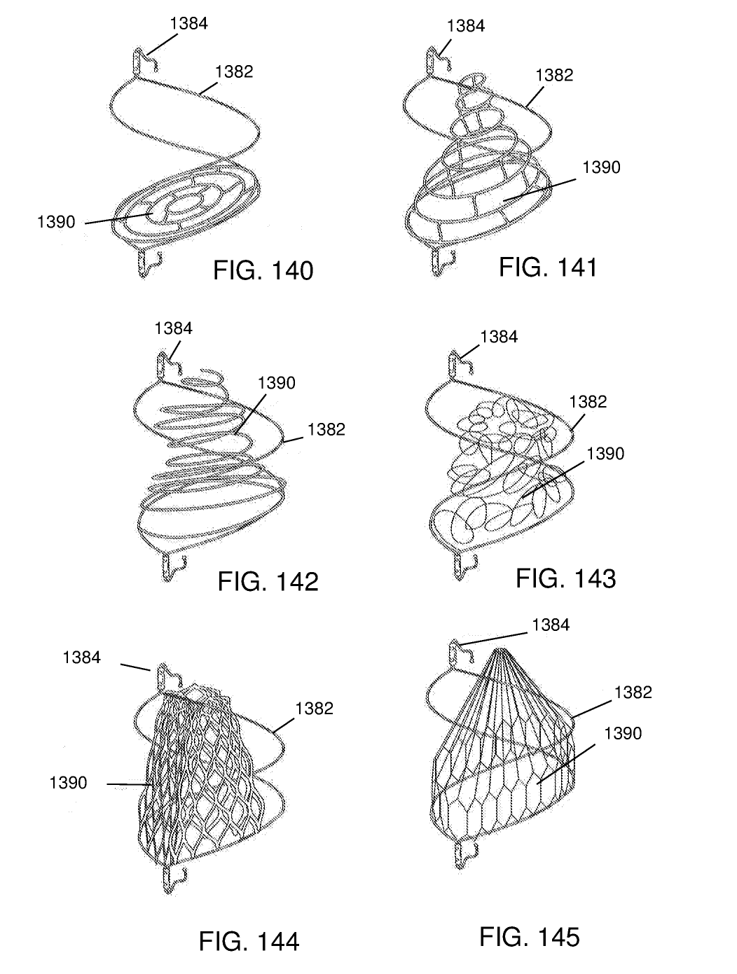

FIG. 140 is a perspective view of an illustrative material capture structure 1390 fabricated using one or more ALM technique. The MCS 1390 has a generally planar shape so as to form a plurality of filter cells generally in the same plane as the support frame used to the support the material capture structure. In this illustrative embodiment, the perimeter of the material capture structure is secured to on loop of a double loop filter support frame using one or more of the techniques illustrated and described in FIGS. 139A-139E.

FIG. 141 is a perspective view of an illustrative material capture structure 1390 fabricated using one or more ALM techniques. The MCS 1390 has a shape so as to form a plurality of filter cells into a generally conical shape extending axially along and through the second loop of the support frame. The material capture structure 1390 is secured to one loop of the double loop filter support frame using one or more of the techniques illustrated and described in FIGS. 139A-139E.

FIG. 142 is a perspective view of an illustrative material capture structure 1390 fabricated using one or more ALM techniques. The MCS 1390 has a form of a variable spiral wound strand pattern into a generally conical shape extended through the second loop of the illustrated double loop structure. The opening of the conical shaped filter is secured to one of the double loops using one or more of the techniques illustrated and described in FIGS. 139A-139E.

FIG. 143 is a perspective view of an illustrative material capture structure 1390 fabricated using one or more ALM techniques. The MCS 1390 has a strand based filter cell arrangement similar to a bird's nest style filter. The filter may be within the double loop frame or extend beyond the frame (as illustrated in the views of FIGS. 141, 142, 144 and 145). A portion of the form is secured to one of the looped filter support frame using one or more of the techniques illustrated and described in FIGS. 139A-139E.

FIG. 144 is a perspective view of an illustrative material capture structure 1390 fabricated using one or more ALM techniques. The MCS 1390 has a plurality of regular spaced filter cells in the form of a truncated cone. In the illustrated embodiment the open end of the truncated cone is secured to one loop of the double loop filter support frame using one or more of the techniques illustrated and described in FIGS. 139A-139E. The truncated cone filter may be within the double loop frame or extend beyond the frame as illustrated in the various other embodiments described herein.

FIG. 145 is a perspective view of an illustrative material capture structure 1390 fabricated using one or more ALM techniques. The MCS 1390 has a hybrid filter cell configuration of one filter cell shape around the perimeter and then a second different filter cell shape extending therefrom. In this embodiment, a portion of the perimeter of the material capture structure 1390 is secured to one loop of the double loop filter support frame using one or more of the techniques illustrated and described in FIGS. 139A-139E. The truncated cone filter may be within the double loop frame or extend beyond the frame as illustrated in the various other embodiments described herein.

It is to be appreciated that through the use of one or more of the above ALM or laser cutting techniques, we have described how to can create a wide array of uniquely configured material captures structures, components, structures, grafts, filters, endoluminal or implantable devices. In some aspects, a material capture structure may be configured with integral connection component to attach to a wireform, support frame or other components without knots. In other words, loops, bands, segments and other attachment means are now integrally formed structures of the design. In addition or alternatively, any of the processes described herein may pause during one or more manufacturing (e.g., ALM) steps to attach one or more components to the medical textile, filter or device being fabricated. In one aspect, the device or element incorporated is one or more radio-opaque or echogenic elements. In one specific embodiment an echogenic or radio opaque marker is incorporated into, on or within a strand formed as described herein as part of a filter or medical textile. Exemplary additional details of materials and designs are provided in U.S. Prov. Pat. App. No. 61/785,955, filed Mar. 14, 2013, titled “ENDOLUMINAL FILTER HAVING ENHANCED ECHOGENIC PROPERTIES,” which is incorporated herein by reference in its entirety. In still other alternatives, a material capture structure is designed as described herein and fabricated with integrally formed fixation elements. With ALM techniques herein we can also customize components (e.g. crimps, anchors, wireforms, support frames) into configurations not possible with other fabrication techniques. Examples of other various configurations and designs of the various embodiments herein include integral crimp and anchor or fixation element, a low profile crimp, an integral wireform and anchor and retrieval feature and other alternatives.

It will be appreciated that material capture structures, as described above, can be formed using more than one of the methods described above. For example, in some embodiments, a material capture structure can comprise a combination of laser cut and 3-D printed features. For example, a main filter portion can be laser cut while the attachments around the perimeter of the filter can be 3-D printed and attached to the laser cut filter portion.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the various embodiments of the present invention.

It is understood that this disclosure, in many respects, is only illustrative of the numerous alternative filtering device embodiments of the present invention. Changes may be made in the details, particularly in matters of shape, size, material and arrangement of various filtering device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention.

What is claimed is:

1. An endoluminal filter, comprising:
a first support member having a first end and a second end;
a second support member attached to the first end of the first support member or the second end of the first support member and forming a crossover with the first support member;
a material capture structure extending between the first and second support members, the crossover and the first end or the second end of the first support member;
at least one tissue anchor on the first support member or the second support member; and
a radiopaque material disposed on at least a portion of the material capture structure.

2. The endoluminal filter of claim 1, wherein the material capture structure comprises a plurality of filaments and the radiopaque material is positioned at an intersection of two or more filaments.

3. The endoluminal filter of claim 2, wherein the radiopaque material comprises a marker bead.

4. The endoluminal filter of claim 3, wherein the marker bead comprises at least one lumen through which at least one filament is configured to be inserted.

5. The endoluminal filter of claim 4, wherein the marker bead comprises two lumens.

6. The endoluminal filter of claim 4, wherein at least a portion of a surface of the at least one lumen comprises a material configured to be bonded to the at least one filament.

7. The endoluminal filter of claim 4, wherein the marker bead comprises at least one protrusion positioned at an opening of the at least one lumen and through which the at least one filament is configured to be inserted.

8. The endoluminal filter of claim 7, wherein at least a portion of the protrusion is configured to be bonded to the at least one filament.

9. The endoluminal filter of claim 7, wherein an interior surface of the protrusion includes ridges and is configured to be swaged to the at least one filament.

10. The endoluminal filter of claim 3, wherein the marker bead is capable of eluting a drug.

11. The endoluminal filter of claim 1, wherein the material capture structure comprises a plurality of filaments and at least a portion of one of the plurality of filaments is doped with a radiopaque material.

12. The endoluminal filter of claim 1, wherein the radiopaque material comprises at least one of tungsten, tantalum, gold, barium, bismuth, platinum, and palladium.

13. The endoluminal filter of claim 1, further comprising an echogenic marker disposed on at least a portion of the material capture structure.

14. An endoluminal filter, comprising:
a first support member having a first end and a second end;
a second support member attached to the first end of the first support member or the second end of the first support member and forming a crossover with the first support member;
a material capture structure extending between the first and second support members, the crossover and the first end or the second end of the first support member, the material capture structure comprising a sheet of material comprising cutouts;
at least one attachment positioned on a perimeter of the material capture structure, the at least one attachment configured to join the material capture structure to the first support member and the second support member; and
at least one tissue anchor on the first support member or the second support member.

15. An endoluminal filter, comprising:
a material capture structure formed using an additive manufacturing technique having a filter cell shape and size for use in filtering particulates no more than 3 mm in any dimension;
a filter support frame sized for use within the vasculature of a mammal; and
a filter securing element on the material capture structure adapted and configured to be coupled to the filter support frame so that in use the material capture structure extends across an opened support frame.

16. The endoluminal filter of claim 15 having a filter cell shape and size for use in filtering particulates no more than 1 mm in any dimension.

17. The endoluminal filter of claim 15 having a filter cell shape and size for use in filtering particulates no more than 200 microns, 0.1 mm, 1 mm, 2 mm, 3 mm, 4 mm, 5 mm, 6 mm, 7 mm, 8 mm in any dimension.

18. The endoluminal filter of claim 15 wherein the filter securing element is integrally formed as a part of the material capture structure.

19. The endoluminal filter of claim 15, wherein the filter securing element further comprising a filter structure attachment, a frame attachment and a spacer joining the filter structure attachment to the frame attachment.

20. The endoluminal filter of claim 17 wherein the filter securing element is formed using an additive manufacturing technique as separate element or integral to the material capture structure.

21. The endoluminal filter of claim 14 wherein the material capture structure is one of a printed pattern or a woven pattern.

22. An endoluminal filter, comprising:
a first support member having a first end and a second end;
a second support member attached to the first end of the first support member or the second end of the first support member and forming a crossover with the first support member;
a material capture structure extending between the first and second support members, the crossover and the first end or the second end of the first support member, the material captures structure formed using an additive manufacturing technique; and
at least one attachment positioned on a perimeter of the material capture structure, attachment configured to join the material capture structure to the first support member and the second support member.

23. The filter of claim 22, wherein the attachment is configured to be tied to the first support member or the second support member.

24. The filter of claim 22, wherein the attachment comprises a tab configured to fold over the first support member or second support member.
